SEQUENCE LISTING (91 sequences)

SEQ ID NO: 1 - protein, 66 aa, Sulfolobus acidocaldarius
MVKVKFKYKGEEKEVDTSKIKKVWRVGKMVSFTYDDNGKTGRGAVSEKDAPKELLDMLARAEREKK

SEQ ID NO: 2 - protein, 64 aa, Saccharolobus solfataricus
MATVKFKYKGEEKEVDISKIKKVWRVGKMISFTYDEGGGKTGRGAVSEKDAPKELLQMLEKQKK

SEQ ID NO: 3 - protein, 65 aa, Sulfolobus acidocaldarius
MAKVRFKYKGEEKEVDTSKIKKVWRVGKMVSFTYDDNGKTGRGAVSEKDAPKELMDMLARAEKKK

SEQ ID NO: 4 - protein, 64 aa, Saccharolobus shibatae
MVTVKFKYKGEEKEVDTSKIKKVWRVGKMISFTYDEGGGKTGRGAVSEKDAPKELLQMLEKQKK

SEQ ID NO: 5 - protein, 64 aa, Sulfolobus shibatae
MATVKFKYKGEEKQVDISKIKKVWRVGKMISFTYDEGGGKTGRGAVSEKDAPKELLQMLEKQKK

SEQ ID NO: 6 - protein, 64 aa, Sulfolobus tokodaii
MVTVKFKYKGEEKEVDISKIKKVWRVGKMISFTYDDNGKTGRGAVSEKDAPKELLQMLEKSGKK

SEQ ID NO: 7 - protein, 64 aa, Sulfolobus islandicus
MVTVKFKYKGEEKQVDTSKIKKVWRVGKMISFTYDEGGGKTGRGAVSEKDAPKELLQMLEKQKK

SEQ ID NO: 8 - protein, 64 aa, Sulfolobus islandicus
MTTVKFKYKGEEKQVDTSKIKKVWRVGKMISFTYDEGGGKTGRGAVSEKDAPKELLQMLEKQKK

SEQ ID NO: 9 - protein, 64 aa, Sulfolobus islandicus
MTTVKFKYKGEEKQVDISKIKKVWRVGKMISFTYDEGGGKTGRGAVSEKDAPKELLQMLEKQKK

SEQ ID NO: 10 - protein, 62 aa, Metallosphaera sedula
MATKIKFKYKGQDLEVDISKVKKVWKVGKMVSFTYDDNGKTGRGAVSEKDAPKELLNMIGKK

SEQ ID NO: 11 - protein, 62 aa, Metallosphaera cuprina
MATKIKFKYKGQDLEVDISKVKKVWKVGKMVSFTYDDNGKTGRGAVSEKDAPKELLSMIGKK

SEQ ID NO: 12 - protein, 61 aa, Acidianus hospitalis
MTTVKFKYKGEEKEVDISKIKKVWRVGKMISFTYDDNGKTGRGAVSEKDAPKELLEKLEKK

SEQ ID NO: 13 - protein, 62 aa, Acidianus hospitalis
MATKVKFKYKGEEKEVDISKIKKVWRVGKMISFTYDDNGKTGRGAVSEKDAPKELLDKLEKK

SEQ ID NO: 14 - protein, 60 aa, Acidianus hospitalis
MATKVKFKYKGEEKEVDISKIKKVWRVGKMISFTYDDNGKTGRGAVSEKDAPKELLEKLK

SEQ ID NO: 15 - protein, 64 aa, Sulfurisphaera tokodaii; identical to SEQ ID NO: 6

SEQ ID NO: 16 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (15)..(15): E or Q
  MOD_RES (18)..(18): T or I
  MOD_RES (31)..(31): V or I
  MOD_RES (37)..(39): This region may encompass one of the following sequences: “EGG” or “DN”
  MOD_RES (57)..(57): M or L
  MOD_RES (58)..(58): Q, D or E
  MOD_RES (59)..(59): M or K
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFKYKGEEKXVDXSKIKKVWRVGKMXSFTYDXXXGKTGRGAVSEKDAPKELXXXLXXXXXXXX

SEQ ID NO: 17 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (8)..(10): Any amino acid
  MOD_RES (15)..(15): E or Q
  MOD_RES (17)..(17): D or E
  MOD_RES (18)..(18): T or I
  MOD_RES (22)..(23): Any amino acid
  MOD_RES (27)..(27): Any amino acid
  MOD_RES (30)..(30): Any amino acid
  MOD_RES (31)..(31): V or I
  MOD_RES (32)..(32): A, I, L, V, Y, M, F or H
  MOD_RES (34)..(34): S, T or A
  MOD_RES (37)..(39): This region may encompass one of the following sequences: “EGG” or “DN” or “DQ”
  MOD_RES (42)..(42): T or I
  MOD_RES (46)..(46): A, I, L, V, Y, M, F or H
  MOD_RES (48)..(48): Any amino acid
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFXXXGEEKXVXXSKIXXVYRXGKXXXFXYDXXXGKXGWGXVXEKDAPKELXXXLXXXXXXXX

SEQ ID NO: 18 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (8)..(9): Any amino acid
  MOD_RES (15)..(15): E or Q
  MOD_RES (17)..(17): D or E
  MOD_RES (18)..(18): T or I
  MOD_RES (22)..(23): Any amino acid
  MOD_RES (27)..(27): Any amino acid
  MOD_RES (30)..(30): Any amino acid
  MOD_RES (31)..(31): V or I
  MOD_RES (34)..(34): S, T or A
  MOD_RES (37)..(39): This region may encompass one of the following sequences: “EGG” or “DN” or “DQ”
  MOD_RES (42)..(42): T or I
  MOD_RES (48)..(48): Any amino acid
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFXXTGEEKXVXXSKIXXVYRXGKXXLFXYDXXXGKXGWGYVXEKDAPKELXXXLXXXXXXXX

SEQ ID NO: 19 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (8)..(8): Any amino acid
  MOD_RES (15)..(15): E or Q
  MOD_RES (17)..(17): D or E
  MOD_RES (18)..(18): T or I
  MOD_RES (22)..(22): Any amino acid
  MOD_RES (23)..(23): Any amino acid
  MOD_RES (27)..(27): Any amino acid
  MOD_RES (31)..(31): V or I
  MOD_RES (37)..(39): This region may encompass one of the following sequences: “EGG” or “DN” or “DQ”
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFXATGEEKXVXXSKIXXVYRXGKDXLFSYDXXXGKIGWGYVSEKDAPKELXXXLXXXXXXXX

SEQ ID NO: 20 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (15)..(15): E or Q
  MOD_RES (17)..(17): D or E
  MOD_RES (18)..(18): T or I
  MOD_RES (31)..(31): V or I
  MOD_RES (37)..(39): This region may encompass one of the following sequences: “EGG” or “DN” or “DQ”
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFDATGEEKXVXXSKISAVYRTGKDXLFSYDXXXGKIGWGYVSEKDAPKELXXXLXXXXXXXX

SEQ ID NO: 21 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (15)..(15): E or Q
  MOD_RES (17)..(17): D or E
  MOD_RES (18)..(18): T or I
  MOD_RES (31)..(31): V or I
  MOD_RES (37)..(39): This region may encompass one of the following sequences: “EGG” or “DN” or “DQ”
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFAATGEEKXVXXSKIANVYRVGKDXLFSYDXXXGKIGWGYVSEKDAPKELXXXLXXXXXXXX

SEQ ID NO: 22 - protein, 64 aa, Artificial Sequence
  Description of Artificial Sequence: Synthetic polypeptide
  MOD_RES (7)..(9): Any amino acid
  MOD_RES (16)..(16): E or D
  MOD_RES (21)..(22): Any amino acid
  MOD_RES (26)..(26): Any amino acid
  MOD_RES (29)..(29): Any amino acid
  MOD_RES (31)..(31): A, I, L, V, Y, M, F or H
  MOD_RES (33)..(33): S, T or A
  MOD_RES (37)..(37): N or Q
  MOD_RES (40)..(40): T or I
  MOD_RES (44)..(44): A, I, L, V, Y, M, F or H
  MOD_RES (46)..(46): Any amino acid
  MOD_RES (57)..(57): M or L
  See specification as filed for detailed description of substitutions and preferred embodiments
MVKVKFXXXGEEKEVXTSKIXXVYRXGKXVXFXYDDXGKXGWGXVXEKDAPKELLDXLARAERE

SEQ ID NO: 23 - protein, 64 aa, Artificial Sequence
  Description of Artificial Sequence: Synthetic polypeptide
  MOD_RES (7)..(8): Any amino acid
  MOD_RES (16)..(16): E or D
  MOD_RES (21)..(22): Any amino acid
  MOD_RES (26)..(26): Any amino acid
  MOD_RES (29)..(29): Any amino acid
  MOD_RES (33)..(33): S, T or A
  MOD_RES (37)..(37): N or Q
  MOD_RES (40)..(40): T or I
  MOD_RES (46)..(46): Any amino acid
  MOD_RES (57)..(57): M or L
  See specification as filed for detailed description of substitutions and preferred embodiments
MVKVKFXXTGEEKEVXTSKIXXVYRXGKXVLFXYDDXGKXGWGYVXEKDAPKELLDXLARAERE

SEQ ID NO: 24 - protein, 64 aa, Artificial Sequence
  Description of Artificial Sequence: Synthetic polypeptide
  MOD_RES (7)..(7): Any amino acid
  MOD_RES (16)..(16): E or D
  MOD_RES (21)..(22): Any amino acid
  MOD_RES (26)..(26): Any amino acid
  MOD_RES (37)..(37): N or Q
  MOD_RES (57)..(57): M or L
  See specification as filed for detailed description of substitutions and preferred embodiments
MVKVKFXATGEEKEVXTSKIXXVYRXGKDVLFSYDDXGKIGWGYVSEKDAPKELLDXLARAERE

SEQ ID NO: 25 - protein, 64 aa, Artificial Sequence
  Description of Artificial Sequence: Synthetic polypeptide
  MOD_RES (7)..(9): Any amino acid
  MOD_RES (16)..(16): E or D
  MOD_RES (21)..(22): Any amino acid
  MOD_RES (26)..(26): Any amino acid
  MOD_RES (29)..(29): Any amino acid
  MOD_RES (31)..(31): A, I, L, V, Y, M, F or H
  MOD_RES (33)..(33): S, T or A
  MOD_RES (41)..(41): T or I
  MOD_RES (45)..(45): A, I, L, V, Y, M, F or H
  MOD_RES (47)..(47): Any amino acid
  MOD_RES (58)..(58): M or L
  See specification as filed for detailed description of substitutions and preferred embodiments
MATVKFXXXGEEKEVXISKIXXVYRXGKXIXFXYDEGGGKXGWGXVXEKDAPKELLQXLEKQKK

SEQ ID NO: 26 - protein, 63 aa, Artificial Sequence
  Description of Artificial Sequence: Synthetic polypeptide
  MOD_RES (7)..(7): Any amino acid
  MOD_RES (16)..(16): E or D
  MOD_RES (21)..(21): A, S, T, V, I, L, Y, or F
  MOD_RES (22)..(22): Any amino acid
  MOD_RES (26)..(26): S, T, V, I, L, Y, or F
  MOD_RES (58)..(58): M or L
  See specification as filed for detailed description of substitutions and preferred embodiments
MATVKFXATGEEKEVXISKIXXVYRXGKDILFSYDEGGGKIGWGYVSEKDAPKELLQXEKQKK

SEQ ID NO: 27 - protein, 65 aa, Artificial Sequence
  Description of Artificial Sequence: Synthetic polypeptide
MVKVKFEATGEEKEVDTSKIYQVYRSGKEVLFTYDDNGKTGWGYVSEKDAPKELLDMLARAEREK

SEQ ID NO: 28-32 - skipped (5 entries)

SEQ ID NO: 33 - protein, 65 aa, Artificial Sequence
  Description of Artificial Sequence: Synthetic polypeptide
MVKVKFAATGEEKEVETSKIANVYRVGKDVLFSYDDQGKIGWGYVSEKDAPKELLDLLARAEREK

SEQ ID NO: 34 - protein, 65 aa, Artificial Sequence
  Description of Artificial Sequence: Synthetic polypeptide
MVKVKFDATGEEKEVETSKISAVYRTGKDVLFSYDDQGKIGWGYVSEKDAPKELLDLLARAEREK

SEQ ID NO: 35 - protein, 214 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 231 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 37 - protein, 293 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGSGSAGSGSGGSVKVKFDATGEEKEVETSKISAVYRTGKDVLFSYDDQGKIGWGYVSEKDAPKELLDLLARAEREK

SEQ ID NO: 38 - protein, 309 aa, Artificial Sequence
  Description of Artificial Sequence: Synthetic polypeptide
EVQLVESGGGLVQPGGSLRLSCAASGYDFTHYGMWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGSGGSGSAGSGSGGSVKVKFDATGEEKEVETSKISAVYRTGKDVLFSYDDQGKIGWGYVSEKDAPKELLDLLARAEREK

SEQ ID NO: 39 - protein, 293 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGSGSAGSGSGGSVKVKFEATGEEKEVDTSKIYQVYRSGKEVLFTYDDNGKTGWGYVSEKDAPKELLDMLARAEREK

SEQ ID NO: 40 - protein, 310 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGSGGSGSAGSGSGGSVKVKFEATGEEKEVDTSKIYQVYRSGKEVLFTYDDNGKTGWGYVSEKDAPKELLDMLARAEREK

SEQ ID NO: 41 - protein, 15 aa, Artificial Sequence
  Description of Artificial Sequence: Synthetic peptide
GSGGSGSAGSGSGGS

SEQ ID NO: 42 - protein, 12 aa, Artificial Sequence
  Description of Artificial Sequence: Synthetic peptide
GSAGSAAGSGEF

SEQ ID NO: 43 - protein, 14 aa, Artificial Sequence
  Description of Artificial Sequence: Synthetic peptide
EGKSSGSGSESKST

SEQ ID NO: 44 - protein, 9 aa, Artificial Sequence
  Description of Artificial Sequence: Synthetic peptide
EPKSCDKTH

SEQ ID NO: 45 - skipped

SEQ ID NO: 46 - protein, 10 aa, Artificial Sequence
  Description of Artificial Sequence: Synthetic peptide
WESNGQPENN

SEQ ID NO: 47 - protein, 17 aa, Artificial Sequence
  Description of Artificial Sequence: Synthetic peptide
KTISKAKGQPREPQVYT

SEQ ID NO: 48-50 - skipped (3 entries)

SEQ ID NO: 51 - protein, 62 aa, Artificial Sequence
  Description of Artificial Sequence: Synthetic polypeptide
MATVKFDATGEEKEVEISKISAVYRTGKDILFSYDEGGGKIGWGYVSEKDAPKELLQLLEKQ

SEQ ID NO: 52 - protein, 62 aa, Artificial Sequence
  Description of Artificial Sequence: Synthetic polypeptide
MATVKFAATGEEKEVEISKIANVYRVGKDILFSYDEGGGKIGWGYVSEKDAPKELLQLLEKQ

SEQ ID NO: 53 - protein, 13 aa, Artificial Sequence
  Description of Artificial Sequence: Synthetic peptide
ICVVQDWGHHRCT

SEQ ID NO: 54 - skipped

SEQ ID NO: 55 - protein, 293 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGSGSAGSGSGGSVKVKFAATGEEKEVETSKIANVYRVGKDVLFSYDDQGKIGWGYVSEKDAPKELLDLLARAEREK

SEQ ID NO: 56 - protein, 310 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGSGGSGSAGSGSGGSVKVKFAATGEEKEVETSKIANVYRVGKDVLFSYDDQGKIGWGYVSEKDAPKELLDLLARAEREK

SEQ ID NO: 57 - protein, 292 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGSGSAGSGSGGSVKVKFDATGEEKEVETSKISAVYRTGKDVLFSYDDQGKIGWGYVSEKDAPKELLDLLARAERE

SEQ ID NO: 58 - protein, 309 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGSGGSGSAGSGSGGSVKVKFDATGEEKEVETSKISAVYRTGKDVLFSYDDQGKIGWGYVSEKDAPKELLDLLARAERE

SEQ ID NO: 59 - protein, 309 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGSGGSGSAGSGSGGSVKVKFVFSGEEKEVETSKIKWVLRWGKAVGFKYDDQGKIGYGFVAEKDAPKELLDLLARAERE

SEQ ID NO: 60 - protein, 371 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGSGSAGSGSGGSVKVKFDATGEEKEVETSKISAVYRTGKDVLFSYDDQGKIGWGYVSEKDAPKELLDLLARAEREKGSGGSGSAGSGSGGSVKVKFVFSGEEKEVETSKIKWVLRWGKAVGFKYDDQGKIGYGFVAEKDAPKELLDLLARAERE

SEQ ID NO: 61 - protein, 388 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGSGGSGSAGSGSGGSVKVKFDATGEEKEVETSKISAVYRTGKDVLFSYDDQGKIGWGYVSEKDAPKELLDLLARAEREKGSGGSGSAGSGSGGSVKVKFVFSGEEKEVETSKIKWVLRWGKAVGFKYDDQGKIGYGFVAEKDAPKELLDLLARAERE

SEQ ID NO: 62 - protein, 371 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGSGSAGSGSGGSVKVKFVFSGEEKEVETSKIKWVLRWGKAVGFKYDDQGKIGYGFVAEKDAPKELLDLLARAEREKGSGGSGSAGSGSGGSVKVKFDATGEEKEVETSKISAVYRTGKDVLFSYDDQGKIGWGYVSEKDAPKELLDLLARAERE

SEQ ID NO: 63 - protein, 388 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGSGGSGSAGSGSGGSVKVKFVFSGEEKEVETSKIKWVLRWGKAVGFKYDDQGKIGYGFVAEKDAPKELLDLLARAEREKGSGGSGSAGSGSGGSVKVKFDATGEEKEVETSKISAVYRTGKDVLFSYDDQGKIGWGYVSEKDAPKELLDLLARAERE

SEQ ID NO: 64 - protein, 66 aa, Artificial Sequence
  Description of Artificial Sequence: Synthetic polypeptide
  MOD_RES (16)..(16): D or E
  MOD_RES (37)..(37): N or Q
  MOD_RES (57)..(57): M or L
MVKVKFVFSGEEKEVXTSKIKWVLRWGKAVGFKYDDXGKIGYGFVAEKDAPKELLDXLARAEREKK

SEQ ID NO: 65 - protein, 66 aa, Artificial Sequence
  Description of Artificial Sequence: Synthetic polypeptide
  MOD_RES (7)..(9): Any amino acid
  MOD_RES (16)..(16): Any amino acid
  MOD_RES (21)..(22): Any amino acid
  MOD_RES (24)..(24): Any amino acid
  MOD_RES (26)..(26): Any amino acid
  MOD_RES (29)..(29): Any amino acid
  MOD_RES (31)..(31): Any amino acid
  MOD_RES (37)..(37): Any amino acid
  MOD_RES (40)..(40): Any amino acid
  MOD_RES (42)..(42): Any amino acid
  MOD_RES (44)..(44): Any amino acid
  MOD_RES (46)..(46): Any amino acid
MVKVKFXXXGEEKEVXTSKIXXVXRXGKXVXFTYDDXGKXGXGXVXEKDAPKELLDMLARAEREKK

SEQ ID NO: 66 - protein, 66 aa, Artificial Sequence
  Description of Artificial Sequence: Synthetic polypeptide
  MOD_RES (7)..(9): Any amino acid
  MOD_RES (16)..(16): Any amino acid
  MOD_RES (21)..(22): Any amino acid
  MOD_RES (24)..(24): Any amino acid
  MOD_RES (26)..(26): Any amino acid
  MOD_RES (29)..(29): Any amino acid
  MOD_RES (31)..(31): Any amino acid
  MOD_RES (33)..(33): Any amino acid
  MOD_RES (37)..(37): Any amino acid
  MOD_RES (40)..(40): Any amino acid
  MOD_RES (42)..(42): Any amino acid
  MOD_RES (44)..(44): Any amino acid
  MOD_RES (46)..(46): Any amino acid
  MOD_RES (57)..(57): Any amino acid
MVKVKFXXXGEEKEVXTSKIXXVXRXGKXVXFXYDDXGKXGXGXVXEKDAPKELLDXLARAEREKK

SEQ ID NO: 67 - protein, 10 aa, Artificial Sequence
  Description of Artificial Sequence: Synthetic peptide
EPKSCDKTHL

SEQ ID NO: 68 - protein, 18 aa, Artificial Sequence
  Description of Artificial Sequence: Synthetic peptide
RLIEDICLPRWGCLWEDD

SEQ ID NO: 69 - protein, 20 aa, Artificial Sequence
  Description of Artificial Sequence: Synthetic peptide
QRLMEDICLPRWGCLWEDDF

SEQ ID NO: 70 - protein, 29 aa, Artificial Sequence
  Description of Artificial Sequence: Synthetic peptide
SDFYKRLINKAKTVEGVEALKLHILAALP

SEQ ID NO: 71 - protein, 64 aa, Artificial Sequence
  Description of Artificial Sequence: Synthetic polypeptide
  MOD_RES (15)..(15): D or E
  MOD_RES (36)..(36): N or Q
  MOD_RES (56)..(56): M or L
VKVKFVFSGEEKEVXTSKIKWVLRWGKAVGFKYDDXGKIGYGFVAEKDAPKELLDXLARAEREK

SEQ ID NO: 72 - protein, 64 aa, Artificial Sequence
  Description of Artificial Sequence: Synthetic polypeptide
VKVKFVFSGEEKEVDTSKIKWVLRWGKAVGFKYDDNGKIGYGFVAEKDAPKELLDMLARAEREK

SEQ ID NO: 73 - protein, 64 aa, Artificial Sequence
  Description of Artificial Sequence: Synthetic polypeptide
VKVKFVFSGEEKEVETSKIKWVLRWGKAVGFKYDDQGKIGYGFVAEKDAPKELLDLLARAEREK

SEQ ID NO: 74 - protein, 10 aa, Artificial Sequence
  Description of Artificial Sequence: Synthetic peptide
RYPISRPRKR

SEQ ID NO: 75 - protein, 12 aa, Artificial Sequence
  Description of Artificial Sequence: Synthetic peptide
GAHWQFNALTVR

SEQ ID NO: 76 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (15)..(15): E or Q
  MOD_RES (17)..(17): D or E
  MOD_RES (18)..(18): T or I
  MOD_RES (31)..(31): V or I
  MOD_RES (37)..(39): This region may encompass one of the following sequences: “EGG” or “DN” or “DQ”
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFKYKGEEKXVXXSKIKKVWRVGKMXSFTYDXXXGKTGRGAVSEKDAPKELXXXLXXXXXXXX

SEQ ID NO: 77 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (8)..(10): Any amino acid
  MOD_RES (15)..(15): E or Q
  MOD_RES (17)..(17): D or E
  MOD_RES (18)..(18): T or I
  MOD_RES (22)..(23): Any amino acid
  MOD_RES (25)..(25): Any amino acid
  MOD_RES (27)..(27): Any amino acid
  MOD_RES (30)..(30): Any amino acid
  MOD_RES (31)..(31): V or I
  MOD_RES (32)..(32): Any amino acid
  MOD_RES (34)..(34): Any amino acid
  MOD_RES (37)..(39): This region may encompass one of the following sequences: “EGG” or “DN” or “DQ”
  MOD_RES (42)..(42): Any amino acid
  MOD_RES (44)..(44): Any amino acid
  MOD_RES (46)..(46): Any amino acid
  MOD_RES (48)..(48): Any amino acid
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
  See specification as filed for detailed description of substitutions and preferred embodiments
MXXXVXFXXXGEEKXVXXSKIXXVXRXGKXXXFXYDXXXGKXGXGXVXEKDAPKELXXXLXXXXXXXX

SEQ ID NO: 78 - protein, 68 aa, Artificial Sequence
  Description of Artificial Sequence: Synthetic polypeptide
  MOD_RES (2)..(2): V, A or T
  MOD_RES (3)..(3): T or K
  MOD_RES (4)..(4): K or absent
  MOD_RES (6)..(6): R or K
  MOD_RES (8)..(8): E, A, or D
  MOD_RES (15)..(15): E or Q
  MOD_RES (17)..(17): D or E
  MOD_RES (18)..(18): T or I
  MOD_RES (22)..(22): Y, A, or S
  MOD_RES (23)..(23): Q, N, or A
  MOD_RES (27)..(27): S, V, or T
  MOD_RES (30)..(30): E or D
  MOD_RES (31)..(31): V or I
  MOD_RES (32)..(32): A, I, L, V, Y, M, F or H
  MOD_RES (34)..(34): A, T or S
  MOD_RES (37)..(39): This region may encompass one of the following sequences: “EGG” or “DN” or “DQ”
  MOD_RES (42)..(42): T or I
  MOD_RES (46)..(46): A, I, L, V, Y, M, F or H
  MOD_RES (57)..(57): M or L
  MOD_RES (58)..(58): Q, D or E
  MOD_RES (59)..(59): M, K, or L
  MOD_RES (61)..(68): This region may encompass one of the following sequences: “K” or “EKSGKK” or “EKQKK” or “ARAEREKK” or “ARAEREK” or “ARAERE” or “ARAEKKK” or “EKK” or “EKSGKK” or “ACAEREKK” or “ACAEKKK”
MXXXVXFXATGEEKXVXXSKIXXVYRXGKXXXFXYDXXXGKXGWGXVSEKDAPKELXXXLXXXXXXXX

SEQ ID NO: 79 - protein, 8 aa, Artificial Sequence
  Description of Artificial Sequence: Synthetic peptide
ARAEREKK

SEQ ID NO: 80 - protein, 6 aa, Artificial Sequence
  Description of Artificial Sequence: Synthetic peptide
ARAERE

SEQ ID NO: 81 - protein, 7 aa, Artificial Sequence
  Description of Artificial Sequence: Synthetic peptide
ARAEREK

SEQ ID NO: 82 - protein, 6 aa, Artificial Sequence
  Description of Artificial Sequence: Synthetic peptide
EKSGKK

SEQ ID NO: 83 - protein, 5 aa, Artificial Sequence
  Description of Artificial Sequence: Synthetic peptide
EKQKK

SEQ ID NO: 84 - protein, 7 aa, Artificial Sequence
  Description of Artificial Sequence: Synthetic peptide
ARAEKKK

SEQ ID NO: 85 - protein, 8 aa, Artificial Sequence
  Description of Artificial Sequence: Synthetic peptide
ACAEREKK

SEQ ID NO: 86 - protein, 7 aa, Artificial Sequence
  Description of Artificial Sequence: Synthetic peptide
ACAEKKK

SEQ ID NO: 87 - protein, 25 aa, Artificial Sequence
  Description of Artificial Sequence: Synthetic peptide
  SITE (1)..(25): This sequence may encompass 1-5 “Gly Gly Gly Gly Ser” repeating units
GGGGSGGGGSGGGGSGGGGSGGGGS

SEQ ID NO: 88 - protein, 25 aa, Artificial Sequence
  Description of Artificial Sequence: Synthetic peptide
  SITE (1)..(25): This sequence may encompass 1-5 “ Ser Gly Gly Gly Gly” repeating units
SGGGGSGGGGSGGGGSGGGGSGGGG

SEQ ID NO: 89 - protein, 25 aa, Artificial Sequence
  Description of Artificial Sequence: Synthetic peptide
  SITE (1)..(25): This sequence may encompass 5-25 residues
GGGGGGGGGGGGGGGGGGGGGGGGG

SEQ ID NO: 90 - protein, 5 aa, Artificial Sequence
  Description of Artificial Sequence: Synthetic peptide
  MOD_RES (4)..(4): Any amino acid except Proline
VPGXG

SEQ ID NO: 91 - protein, 11 aa, Artificial Sequence
  Description of Artificial Sequence: Synthetic peptide
DICLPRWGCLW

(19) United States
(12) Patent Application Publication
(10) Pub. No.: US 2025/0145673 A1
(43) Pub. Date: May 8, 2025

(54) POLYPEPTIDES FOR COMPLEMENT INHIBITION

(71) Applicants: Affilogic, Nantes (FR); Apellis Pharmaceuticals, Inc., Waltham, MA (US)

(72) Inventors: Mathieu Cinier, Nantes (FR); Anne Chevrel; Olivier Kitten; Lukas Scheibler, Telluride, CO (US)

(21) Appl. No.: 18/282,664

(22) PCT Filed: Mar. 18, 2022
(86) PCT No.: PCT/US22/21044

§ 371 Date: Sep. 18, 2023

(30) Foreign Application Priority Data
Mar. 18, 2021 (EP) 21305336.6
Jun. 30, 2021 (EP) 21305904.1
Jun. 30, 2021 (EP) 21305905.8

(51) Int. Cl. C07K 14/195; A61K 38/00; A61K 39/00; C07K 16/22

(52) U.S. Cl. CPC C07K 14/195; C07K 16/22; A61K 38/00; A61K 2039/505; C07K 2317/55; C07K 2317/73; C07K 2317/76; C07K 2317/92; C07K 2317/94; C07K 2319/00

(57) ABSTRACT

Proteins comprising a C3 and/or C3b binding polypeptide and a VEGF inhibitor, and their use in treating complement-mediated disorders, are described.

### CROSS REFERENCE TO RELATED APPLICATIONS

This application is the National Stage of International Application No. PCT/US22/21044 filed Mar. 18, 2022, which claims the benefit of European Patent Application No. 21305905.8 filed Jun. 30, 2021, European Patent Application No. 21305904.1 filed Jun. 30, 2021, and European Patent Application No. 21305336.6 filed Mar. 18, 2021, the contents of each of which are hereby incorporated by reference herein in their entirety.

### SEQUENCE LISTING

The instant application contains a Sequence Listing which has been submitted electronically in ASCII format and is hereby incorporated by reference in its entirety. Said ASCII copy, created on Apr. 26, 2022, is named 2008575-0558_SL.txt and is 94,984 bytes in size.

### BACKGROUND

Complement is a system consisting of more than 30 plasma and cell-bound proteins that plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway. Inappropriate or excessive complement activation is an underlying cause or contributing factor to a number of serious diseases and conditions, and considerable effort has been devoted over the past several decades to exploring various complement inhibitors as therapeutic agents.

### SUMMARY

The present disclosure provides methods and compositions useful, e.g., for treatment of a complement-mediated disorder (e.g., in a subject having or at risk of a complement-mediated disorder) and/or for modulating complement. In one aspect, the present disclosure provides a polypeptide (e.g., a monomer, dimer, or a fusion protein) capable of specifically binding to C3 and/or C3b. In another aspect, the present disclosure provides a polypeptide capable of inhibiting the classical complement pathway and/or alternative complement pathway. In various instances, such a polypeptide is improved in one or more properties (e.g., binding affinity for C3 and/or C3b) as compared to a reference binding molecule, e.g., a reference polypeptide. In certain instances, a reference polypeptide comprises the amino acid sequence of the wild-type Sac7d polypeptide (e.g., comprising an amino acid sequence of SEQ ID NO: 1).

In some aspects, the present disclosure provides bifunctional proteins that (i) specifically bind to C3 and/or C3b and inhibit complement and (ii) specifically bind to VEGF and inhibit angiogenesis. Further provided are methods of using the bifunctional proteins, e.g., to treat disorders in which both complement activation and inappropriate or excessive angiogenesis and/or blood vessel leakage play a role. In some aspects, bifunctional proteins described herein may be administered intravitreally to treat age-related macular degeneration.

In some aspects, the disclosure provides a protein comprising (i) a C3 and/or C3b binding polypeptide and (ii) a VEGF inhibitor, wherein the amino acid sequence of the C3 and/or C3b binding polypeptide has between 4 and 22 substitutions as compared to the Sac7d family consensus sequence of SEQ ID NO: 16, optionally wherein the amino acid sequence of the C3 and/or C3b binding polypeptide comprises amino acids 2-60 of SEQ ID NO:77, wherein X represents any amino acid, and wherein SEQ ID NO:77 comprises at least 3 of the following: E, A, or D at position 8; A at position 9; T at position 10; Y, A, or S at position 22; Q, N, or A at position 23; Y at position 25; S, V, or T at position 27; E or D at position 30; A, I, L, V, Y, M, F or H at position 32; A, T or S at position 34; T or I at position 42; W at position 44; A, I, L, V, Y, M, F or H at position 46; and S at position 48.

In some embodiments, SEQ ID NO:77 comprises Y at position 25, W at position 44, or both. In some embodiments, SEQ ID NO:77 comprises Y at position 25 and W at position 44. In some embodiments, SEQ ID NO:77 comprises T or I at position 42. In some embodiments, SEQ ID NO:77 comprises L at position 32, Tyr at position 46, or both. In some embodiments, SEQ ID NO:77 comprises L at position 32 and Tyr at position 32. In some embodiments, SEQ ID NO:77 comprises A, T or S at position 34. In some embodiments, SEQ ID NO:77 comprises S at position 34, I at position 42, or both. In some embodiments, SEQ ID NO:77 comprises S at position 34 and I at position 42. In some embodiments, SEQ ID NO:77 comprises A at position 9, T at position 10, or both. In some embodiments, SEQ ID NO:77 comprises A at position 9 and T at position 10. In some embodiments, SEQ ID NO:77 comprises S at position 48. In some embodiments, SEQ ID NO:77 comprises E or D at position 30. In some embodiments, SEQ ID NO:77 comprises D at position 30. In some embodiments, SEQ ID NO:77 comprises E, A, or D at position 8. In some embodiments, SEQ ID NO:77 comprises A or D at position 8. In some embodiments, SEQ ID NO:77 comprises Y, A, or S at position 22. In some embodiments, SEQ ID NO:77 comprises A or S at position 22. In some embodiments, SEQ ID NO:77 comprises Q, N, or A at position 23. In some embodiments, SEQ ID NO:77 comprises N or A at position 23. In some embodiments, SEQ ID NO:77 comprises S, V, or T at position 27. In some embodiments, SEQ ID NO:77 comprises V or T at position 27. In some embodiments, SEQ ID NO:77 comprises: Y at position 25; A, I, L, V, Y, M, F or H at position 32; A, T or S at position 34; T or I at position 42; W at position 44; A, I, L, V, Y, M, F or H at position 46; and S at position 48. In some embodiments, SEQ ID NO:77 comprises: Y at position 25; L at position 32; A, T or S at position 34; T or I at position 42; W at position 44; Y at position 46; and S at position 48. In some embodiments, SEQ ID NO:77 comprises: A at position 9; T at position 10; Y at position 25; L at position 32; W at position 44; Y at position 46; and S at position 48. In some embodiments, SEQ ID NO:77 comprises: E, A, or D at position 8; A at position 9; T at position 10; Y, A, or S at position 22; Q, N, or A at position 23; Y at position 25; S, V, or T at position 27; E or D at position 30; L at position 32; A, T or S at position 34; T or I at position 42; W at position 44; Y at position 46; and S at position 48. In some embodiments, SEQ ID NO:77 comprises: A or D at position 8; A at position 9; T at position 10; A or S at position 22; N or A at position 23; Y at position 25; V or T at position 27; D at position 30; L at position 32; S at position 34; I at position 42; W at position 44; Y at position 46; and S at position 48. In some embodiments, SEQ ID NO:77 comprises: A at position 9; T at position 10; Y at position 25; D at position 30; L at position 32; S at position 34; I at position 42; W at position 44; Y at position 46; and S at position 48. In some embodiments, SEQ ID NO:77 comprises: E at position 8; A at position 9; T at position 10; Y at position 22; Q at position 23; Y at position 25; S at position 27; E at position 30; L at position 32; A at position 34; T at position 42; W at position 44; V at position 46; and S at position 48. In some embodiments, SEQ ID NO:77 comprises: A at position 8; A at position 9; T at position 10; A at position 22; N at position 23; Y at position 25; V at position 27; D at position 30; L at position 32; S at position 34; I at position 42; W at position 44; Y at position 46; and S at position 48. In some embodiments, SEQ ID NO:77 comprises: D at position 8; A at position 9; T at position 10; S at position 22; A at position 23; Y at position 25; T at position 27; D at position 30; L at position 32; S at position 34; I at position 42; W at position 44; Y at position 46; and S at position 48.

In some embodiments, the amino acids at positions 2, 3, 4, 6, 15, 17, 18, 31, 37, 38, 39, 57, 58, 59, and 61-68 of SEQ ID NO:77 are the same as those found at the corresponding position in a protein selected from the group consisting of Sac7d from Sulfolobus acidocaldarius, Sac7e from Sulfolobus acidocaldarius, Sso7d from Sulfolobus solfataricus, Ssh7b from Sulfolobus shibatae, Ssh7a from Sulfolobus shibatae, DBP7 from Sulfolobus tokodaii, Sis7a from Sulfolobus islandicus, Mse7 from Metallosphaera sedula, Mcu7 from Metallosphaera cuprina, Aho7a from Acidianus hospitalis, Aho7b from Acidianus hospitalis, Aho7c from Acidianus hospitalis and Sto7 from Sulfurisphaera tokodaii, when said protein is aligned with SEQ ID NO: 77 with the proviso that the protein may comprise a D17E, N38Q, and/or M59L substitution.

In some embodiments, SEQ ID NO:77 comprises the following amino acids at the indicated positions:

| | |
|---|---|
| (2) . . . (2) | X is V |
| (3) . . . (3) | X is K |
| (4) . . . (4) | X is - |
| (6) . . . (6) | X is K |
| (15) . . . (15) | X is E |
| (17) . . . (17) | X is D or E |
| (18) . . . (18) | X is T |
| (31) . . . (31) | X is V |
| (37) . . . (39) | X X X is EGG, DN- or DQ- |
| (57) . . . (57) | X is L |
| (58) . . . (58) | X is D |
| (59) . . . (59) | X is M |
| (61) . . . (68) | XXXXXXXX is ARAEREKK (SEQ ID NO: |
| | 79) or ARAERE-- (SEQ ID NO: 80) |
| | or ARAEREK- (SEQ ID NO: 81) |

In some embodiments, SEQ ID NO:77 comprises E at position 17, Q at position 38, or both. In some embodiments, SEQ ID NO:77 comprises E at position 17 and Q at position 38.

In some embodiments, the C3 and/or C3b binding polypeptide comprises amino acids 2-58 of SEQ ID NO: 27. In some embodiments, the C3 and/or C3b binding polypeptide comprises amino acids 2-58 of SEQ ID NO: 33. In some embodiments, the C3 and/or C3b binding polypeptide comprises amino acids 2-58 of SEQ ID NO: 34.

In some aspects, the disclosure provides a protein comprising (i) a C3 and/or C3b binding polypeptide and (ii) a VEGF inhibitor, wherein the C3 and/or C3b binding polypeptide comprises amino acids 2-58 of the sequence MVKVKFXXXGEEKEVXTSKIXXVXRXGKXVXFTYDDXGKXGXGXVXEKDAPKELLD MLARAEREKK (SEQ ID NO: 65), wherein each X independently represents any amino acid, and wherein SEQ ID NO: 65 comprises: (i) D or E at position 16; (ii) E or Q at position 37; and (iii) Y at position 24, W at position 42, or both.

In some embodiments, SEQ ID NO: 65 comprises Y at position 24 and W at position 42. In some embodiments, SEQ ID NO: 65 comprises: A, I, L, V, Y, M, F or H at position 31; A, T or S at position 33; T or I at position 40; and A, I, L, V, Y, M, F or H at position 44. In some embodiments, SEQ ID NO: 65 comprises L at position 31, Y at position 44, or both. In some embodiments, SEQ ID NO: 65 comprises L at position 31 and Y at position 44. In some embodiments, SEQ ID NO: 65 comprises S at position 33, I at position 40, or both. In some embodiments, SEQ ID NO: 65 comprises S at position 33 and I at position 40. In some embodiments, SEQ ID NO: 65 comprises A at position 8, T at position 9, or both. In some embodiments, SEQ ID NO: 65 comprises A at position 8 and T at position 9. In some embodiments, SEQ ID NO: 6565 comprises S at position 46. In some embodiments, SEQ ID NO: 6565 comprises E or D at position 29. In some embodiments, SEQ ID NO: 65 comprises D at position 29. In some embodiments, SEQ ID NO: 65 comprises E, A, or D at position 7. In some embodiments, SEQ ID NO: 65 comprises A or D at position 7. In some embodiments, SEQ ID NO: 65 comprises Y, A, or S at position 21. In some embodiments, SEQ ID NO: 65 comprises A or S at position 21. In some embodiments, SEQ ID NO: 65 comprises Q, N, or A at position 22. In some embodiments, SEQ ID NO: 65 comprises N or A at position 22. In some embodiments, SEQ ID NO: 65 comprises S, V, or T at position 26. In some embodiments, SEQ ID NO: 65 comprises V or T at position 26. In some embodiments, SEQ ID NO: 65 comprises: E, A, or D at position 7; A at position 8; T at position 9; Y, A, or S at position 21; Q, N, or A at position 22; Y at position 24; S, V, or T at position 26; E or D at position 29; A, I, L, V, Y, M, F or H at position 31; A, T or S at position 33; T or I at position 39; W at position 42; A, I, L, V, Y, M, F or H at position 44; and S at position 46. In some embodiments, SEQ ID NO: 65 comprises: Y at position 24; L at position 31; A, T or S at position 33; T or I at position 40; W at position 42; and Y at position 44. In some embodiments, SEQ ID NO: 65 comprises: A at position 8; T at position 9; Y at position 24; L at position 31; W at position 42; Y at position 44; and S at position 46. In some embodiments, SEQ ID NO: 65 comprises: E, A, or D at position 7; A at position 8; T at position 9; Y, A, or S at position 21; Q, N, or A at position 22; Y at position 24; S, V, or T at position 26; E or D at position 29; L at position 31; A, T or S at position 33; T or I at position 40; W at position 42; Y at position 44; and S at position 46. In some embodiments, SEQ ID NO: 65 comprises: A or D at position 7; A at position 8; T at position 9; A or S at position 21; N or A at position 22; Y at position 24; V or T at position 26; D at position 29; L at position 31; S at position 33; I at position 40; W at position 42; Y at position 44; and S at position 46. In some embodiments, SEQ ID NO: 65 comprises: A at position 8; T at position 9; Y at position 24; D at position 29; L at position 31; S at position 33; I at position 40; W at position 42; Y at position 44; and S at position 46. In some embodiments, SEQ ID NO: 65 comprises: E at position 7; A at position 8; T at position 9; Y at position 21; Q at position 22; Y at position 24; S at position 26; E at position 29; L at position 31; A at position 33; T at position 40; W at position 42; V at position 44; and S at position 46. In some embodiments, SEQ ID NO: 65 comprises: A at position 7; A at position 8; T at position 9; A at position 21; N at position 22; Y at position 24; V at position 26; D at position 29; L at position 31; S at position 33; I at position 40; W at position 42; Y at position 44; and S at position 46. In some embodiments, SEQ ID NO: 65 comprises: D at position 7; A at position 8; T at position 9; S at position 21; A at position 22; Y at position 24; T at position 26; D at position 29; L at position 31; S at position 33; I at position 40; W at position 42; Y at position 44; and S at position 46. In some embodiments, SEQ ID NO: 65 comprises E at position 16, Q at position 37, or both. In some embodiments, SEQ ID NO: 65 comprises E at position 16 and Q at position 37. In some embodiments, SEQ ID NO: 65 comprises the sequence of SEQ ID NO: 27. In some embodiments, SEQ ID NO: 65 comprises the sequence of SEQ ID NO: 33. In some embodiments, SEQ ID NO: 65 comprises the sequence of SEQ ID NO: 34.

In another aspect, the disclosure provides a protein comprising: (i) a C3 and/or C3b binding polypeptide comprising an amino acid sequence that is at least 70% identical to amino acids 2-58 of SEQ ID NO: 1, wherein the amino acid sequence comprises D or A at position 8, A at position 9, T at position 10, E at position 17, S or A at position 22, A or N at position 23, Y at position 25, T or V at position 27, D at position 30, L at position 32, S at position 34, I at position 42, W at position 44, Y at position 46, and S at position 48; and (ii) a VEGF inhibitor.

In another aspect, the disclosure provides a protein comprising (i) a C3 and/or C3b binding polypeptide comprising amino acids 2-58 of any one of SEQ ID NOs: 22, 23, or 24, wherein the amino acid sequence comprises D or A at position 7, A at position 8, T at position 9, S or A at position 21, A or N at position 22, Y at position 24, T or V at position 26, D at position 29, L at position 31, S at position 33, I at position 40, W at position 42, Y at position 44, and S at position 46 and (ii) a VEGF inhibitor.

In another aspect, the disclosure provides a protein comprising (i) C3 and/or C3b binding polypeptide comprising amino acids 2-59 of SEQ ID NO: 25 or 26, wherein the amino acid sequence comprises D or A at position 7, A at position 8, T at position 9, E at position 16, S or A at position 21, A or N at position 22, Y at position 24, T or V at position 26, D at position 29, L at position 31, S at position 33, I at position 41, W at position 43, Y at position 45, and S at position 47; and (ii) a VEGF inhibitor.

In another aspect, the disclosure provides a protein comprising (i) a C3 and/or C3b binding polypeptide comprising an amino acid sequence at least 90% identical to any one of SEQ ID NOs: 27, 33, 34, 51, or 52, or a C3 and/or C3b binding portion thereof and (ii) a VEGF inhibitor.

In some embodiments, the protein comprises an amino acid sequence at least 91%, 92%, 93%, 94%, 95%, 96%, 97%, 98%, or 99% identical to any one of SEQ ID NOs: 27, 33, 34, 51, or 52 or a C3 and/or C3b binding portion thereof. In some embodiments, the protein comprises any one of SEQ ID NOs: 27, 33, 34, 51, or 52, or a C3 and/or C3b binding portion thereof.

In some embodiments, the protein further comprises a second C3 and/or C3b binding polypeptide, optionally wherein the first and second C3 and/or C3b binding polypeptides have the same amino acid sequence. In some embodiments, the second C3 and/or C3b binding polypeptide comprises an amino acid sequence at least 90% identical to any one of SEQ ID NOs: 27, 33, 34, 51, or 52, or a C3 and/or C3b binding portion thereof.

In any of the aspects described herein, some embodiments, the VEGF inhibitor comprises an extracellular domain of a VEGF receptor. In some embodiments, the VEGF inhibitor comprises an antibody fragment selected from the group consisting of a Fab, F(ab′)2, Fv, scFv, or a single domain antibody (e.g., a nanobody). In some embodiments, the antibody fragment is a Fab comprising a VL domain, a CL domain, a VH domain, and a CH1 domain.

In some embodiments, the protein comprises the C3 and/or C3b binding polypeptide fused directly or indirectly to one, two, three, or four of: (i) the VL of the Fab; (ii) the CL of the Fab; (iii) the VH of the Fab; or (iv) the CH1 of the Fab. In some embodiments, the protein comprises the C3 and/or C3b binding polypeptide fused directly or indirectly to the N-terminus of the VL of the Fab, the C-terminus of the CL of the Fab, the N-terminus of the VH of the Fab, or the C-terminus of CH1 of the Fab.

In some embodiments, the protein comprises a linker peptide joining the C3 and/or C3b binding polypeptide and the VEGF inhibitor.

In some embodiments, the antibody fragment comprises a heavy chain fragment having the amino acid sequence of SEQ ID NO: 36 and a light chain having the amino acid sequence of SEQ ID NO: 35, optionally wherein the protein comprises (i) SEQ ID NO: 35 and either SEQ ID NO: 38, SEQ ID NO: 40, or SEQ ID NO: 56 or (ii) SEQ ID NO: 36 and either SEQ ID NO: 37, SEQ ID NO: 39, or SEQ ID NO: 55.

In some embodiments, the antibody fragment is ranibizumab or a fragment of ranibizumab.

In some embodiments, the C3 and/or C3b binding polypeptide or the protein is characterized by a dissociation constant (KD) of between about 0.2 nM to about 100 nM for C3. In some embodiments, the KD about 0.1 nM to about 1 nM or is about 1 nM to about 5 nM or is about 5 nM to about 10 nM. In some embodiments, the C3 and/or C3b binding polypeptide or the protein is characterized by a KD of about 0.1 nM to about 100 nM for C3b. In some embodiments, the C3 and/or C3b binding polypeptide or the protein is characterized by a KD of about 0.1 nM to about 100 nM for C3b, optionally wherein the KD is less than about 100 nM, about 90 nM, about 80 nM, about 70 nM, about 60 nM, about 50 nM, or lower for C3b. In some embodiments, the C3 and/or C3b binding polypeptide or the protein is characterized by an IC50 of about 60 nM to about 1,759 nM for classical complement pathway inhibition. In some embodiments, the C3 and/or C3b binding polypeptide or the protein is characterized by an IC50 of about 60 nM to about 1,759 nM for classical complement pathway inhibition and/or wherein the C3 and/or C3b binding polypeptide or the protein is characterized by an IC50 of about 70 nM to about 700 nM for alternate complement pathway inhibition.

In some embodiments, the protein further comprises a half-life extending polypeptide, optionally wherein the protein has a structure depicted in FIG. 11, FIG. 12, FIG. 13, or FIG. 14.

In another aspect, the disclosure provides a nucleic acid encoding any of the proteins described herein. In another aspect, the disclosure provides an expression vector comprising any of the nucleic acids described herein, optionally wherein the vector is an adeno-associated viral (AAV) vector. In another aspect, the disclosure provides a host cell comprising any expression vector described herein.

In another aspect, the disclosure provides a pharmaceutical composition comprising any of the proteins, nucleic acids, expression vectors, or host cells described herein, and a pharmaceutically acceptable carrier.

In another aspect, the disclosure provides a method of treating a subject having or at risk of a complement-mediated disorder, the method comprising administering to the subject a composition comprising an effective amount of any of the proteins, nucleic acids, expression vectors, host cells, or pharmaceutical compositions described herein. In some embodiments, after the administration of the composition, a level of complement activity in the subject or in a biological sample from the subject is reduced relative to a level before the administration of the composition. In some embodiments, the level of complement activity is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration. In some embodiments, the composition is administered intravenously or subcutaneously to the subject. In some embodiments, the composition is administered to a hepatocyte of the subject.

In some embodiments, the method further comprises administering to the subject a second agent. In some embodiments, the second agent is a compstatin analog or a composition comprising a nucleotide sequence encoding an miRNA or siRNA targeting a C3 transcript. In some embodiments, the second agent comprises a composition comprising a viral vector comprising the nucleotide sequence encoding the miRNA or siRNA. In some embodiments, the viral vector is an adeno-associated viral (AAV) vector. In some embodiments, the AAV vector is an AAV1, AAV2, AAV3 (e.g., AAV3B), AAV4, AAV5, AAV6, AAV7, AAV8, AAV9, AAV10, or AAV11 vector.

In some embodiments, the subject has a defect in complement regulation, optionally wherein the defect comprises abnormally low expression of one or more complement regulatory proteins by at least some of the subject's cells. In some embodiments, the complement-mediated disorder is a chronic disorder. In some embodiments, the complement-mediated disorder involves complement-mediated damage to red blood cells, optionally wherein the disorder is paroxysmal nocturnal hemoglobinuria or atypical hemolytic uremic syndrome. In some embodiments, the complement-mediated disorder is an autoimmune disease, optionally wherein the disorder is multiple sclerosis. In some embodiments, the complement-mediated disorder involves the kidney, optionally wherein the disorder is membranoproliferative glomerulonephritis, lupus nephritis, IgA nephropathy (IgAN), primary membranous nephropathy (primary MN), C3 glomerulopathy (C3G), or acute kidney injury. In some embodiments, the complement-mediated disorder involves the central or peripheral nervous system or neuromuscular junction, optionally wherein the disorder is neuromyelitis optica, Guillain-Barré syndrome, multifocal motor neuropathy, or myasthenia gravis. In some embodiments, the complement-mediated disorder involves the respiratory system, optionally wherein the disorder is characterized by pulmonary fibrosis. In some embodiments, the complement-mediated disorder involves the vascular system, optionally wherein the disorder is characterized by vasculitis.

In some embodiments, the composition is administered to the eye of a subject suffering from an eye disorder. In some embodiments, the composition is administered intravitreally. In some embodiments, the eye disorder is age-related macular degeneration (AMD). In some embodiments, the eye has geographic atrophy. In some embodiments, the eye has wet AMD. In some embodiments, the eye has geographic atrophy and wet AMD. In some embodiments, the eye has intermediate AMD.

### BRIEF DESCRIPTION OF THE DRAWING

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 is an image showing an exemplary Fab fusion protein comprising a Fab (e.g., comprising a heavy chain having the amino acid sequence of SEQ ID NO: 36 and a light chain having the amino acid sequence of SEQ ID NO: 35 (ranibizumab (Lucentis®, Genentech))) and a C3 and/or C3b binding polypeptide described herein (e.g., a polypeptide having the amino acid sequence of SEQ ID NO: 27, SEQ ID NO 33, or SEQ ID NO 34). The polypeptide may be fused to the Fab at position A (CH1), position B (VH), position C (CL), or position D (VL). It is to be understood that Fab fusion proteins of the disclosure can include the polypeptide fused directly or indirectly to the Fab at position A, B, C, or D. For example, “fused to the heavy or light chain of the Fab at position A, B, C, or D” as used herein encompasses direct or indirect fusion at position A, B, C, or D. In some embodiments, a Fab fusion protein includes one or more amino acids between the CH1 domain and the polypeptide at position A, and in such embodiments the polypeptide is “fused to the heavy chain of the Fab at position A” or is “fused to CH1 at position A”, as used herein. In some embodiments, a Fab fusion protein may include a flexible linker between the Fab and polypeptide (e.g., a flexible linker of 15 amino acids in length).

FIG. 2 is a schematic showing the role of C3 in classical, lectin, and alternative pathway complement activation.

FIGS. 3A-3D are a series of graphs showing complement activation as measured using an ELISA-based assay for polypeptides having the amino acid sequence of SEQ ID NO: 27, 33, or 34, and the Fab fusion proteins Fab NF-A, Fab NF-C, and Fab NF-D. Complement activation correlates with the color intensity measured in absorbance (optical density).

FIGS. 4A-4B are a series of graphs showing inhibition of VEGF-induced proliferation by Fab fusion proteins Fab NF-A, Fab NF-C, and Fab NF-D using the classical MTT assay. Lucentis® was used as a control.

FIGS. 5A-5D are graphs showing the results of isothermal titration calorimetry (ITC) to determine the K_{D} of polypeptides (SEQ ID NO:27, 33, or 34) or Fab fusion proteins (NF Fab-A, NF Fab-C, or NF Fab-D) for C3 and C3b.

FIGS. 6A-6F are sensorgrams generated by SPR showing association and dissociation of polypeptides (SEQ ID NO:27, 33, or 34) or Fab fusion proteins (NF Fab-A, NF Fab-C, or NF Fab-D) to C3 and C3b. Dissociation constants (K_{D}) for each polypeptide or Fab fusion protein are also shown.

FIG. 7 is a schematic showing alignment of amino acid sequences of Sac7d family members, which may be used to generate a consensus sequence. Figure discloses SEQ ID NOS 10, 6, 11, 2, 1, 3, 7, 8, 9, 5, 4, 14, 12, 13, and 6, respectively, in order of appearance.

FIG. 8 is an image showing the structure of an exemplary pegylated compstatin analog.

FIG. 9 is a graph of the ratio binding response of C3 and/or C3b binding polypeptides described herein in a competition assay with the exemplary pegylated compstatin analog shown in FIG. 8 (assuming a 10 kD PEG).

FIG. 10 is a schematic showing exemplary Fab fusion proteins comprising a Fab and two, three, or four C3 and/or C3b binding polypeptides. NF represents a C3 and/or C3b binding polypeptide.

FIG. 11 presents schematic diagrams of 6 different structures for exemplary Fab fusion proteins comprising a Fab, a C3 and/or C3b binding polypeptide and a half-life extending polypeptide. NF represents a C3 and/or C3b binding polypeptide. HEP represents a half-life extending polypeptide.

FIG. 12 presents schematic diagrams of 4 different structures for exemplary Fab fusion proteins comprising a Fab, two C3 and/or C3b binding polypeptides, and one half-life extending polypeptide (“HEP”). NF represents a C3 and/or C3b binding polypeptide. HEP represents a half-life extending polypeptide.

FIG. 13 presents schematic diagrams of 4 different structures for exemplary Fab fusion proteins comprising a Fab, one C3 and/or C3b binding polypeptides, and two half-life extending polypeptide. NF represents a C3 and/or C3b binding polypeptide. HEP represents a half-life extending polypeptide.

FIG. 14 presents schematic diagrams of 4 different structures for exemplary Fab fusion proteins comprising a Fab, two C3 and/or C3b binding polypeptides, and two half-life extending polypeptides. NF represents a C3 and/or C3b binding polypeptide. HEP represents a half-life extending polypeptide.

FIG. 15 is a graph showing the inhibition of VEGF-induced proliferation by protein RNF-LA2 or Lucentis® using the MTT assay described in Example 4. The bar on the left in each pair of bars represents Lucentis® while the bar on the right in each pair of bars represents RNF-LA2.

FIG. 16 presents schematic diagrams of 6 different structures for exemplary Fab fusion proteins comprising a Fab, one or two C3 and/or C3b binding polypeptides, and one or two half-life extending polypeptides. NF represents a C3 and/or C3b binding polypeptide. HEP represents a half-life extending polypeptide (e.g., an HSA binding polypeptide).

FIG. 17A-C are a series of graphs showing inhibition of VEGF-induced proliferation by Fab fusion proteins Fab NF-E, Fab NF-F, Fab NF-H, Fab NF-I, and Fab NF-J using the classical MTT assay. Lucentis® was used as a control. The bar on the left in each pair of bars represents Lucentis® while the bar on the right in each pair of bars represents the specific Fab fusion NF protein.

### DEFINITIONS

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically engineered animal, and/or a clone.

Antibody: As used herein, the term “antibody” refers to an immunoglobulin or a derivative thereof containing an immunoglobulin domain capable of binding to an antigen. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxyl-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxyl-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and the tetramer is formed. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments described herein the antibody is a fragment such as a Fab, Fab′, F(ab′)₂, scFv (single-chain variable) or other fragment that retains an antigen binding site. See, e.g., Allen, T., Nature Reviews Cancer, Vol. 2, 750-765, 2002, and references therein. A Fab includes the variable and constant domains of the light chain and the variable domain and CH1 domain of the heavy chain. Generally, a Fab lacks the CH2 and CH3 domains of the heavy chain. In some embodiments, a Fab includes a portion of the hinge region on the carboxyl-terminal end of the CH1 domain. A F(ab′)₂ comprises a pair of Fabs that are generally covalently linked by their hinge cysteines. A Fab′ can be derived from a F(ab′)₂ by cleavage of the hinge disulfide bonds connecting the heavy chains in the F(ab′)₂. Other chemical couplings than disulfide linkages of antibody fragments are also known in the art. In some embodiments, antibody fragments can be derived from an antibody by cleavage of the heavy chain in the hinge region on the N-terminal or C-terminal side of the hinge cysteine residues connecting the heavy chains of the antibody. For example, antibody fragments may be produced by methods known in the art, such as by enzymatic cleavage of an antibody using papain to obtain a Fab or pepsin to obtain a F(ab′)₂. Antibody fragments, such as Fabs, may also be produced recombinantly using techniques that are well known to a person skilled in the art. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present disclosure monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Combination therapy: As used herein, the term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered followed by one or more additional agents. In the separate administration protocol, two or more agents may be administered a few minutes apart, or a few hours apart, a few days apart, or a few weeks apart. In some embodiments, two or more agents may be administered 1-2 weeks apart.

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a polypeptide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., amino acids) as found in the whole polypeptide. In some embodiments, a polypeptide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole polypeptide. The whole material or entity may in some embodiments be referred to as the “parent” of the fragment.

Host cell: As used herein, the term “host cell” refers to a cell into which exogenous nucleic acid, e.g., DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Heterologous: As used herein, the term “heterologous” with respect to a protein or a polypeptide refers to a protein or polypeptide that is non-naturally occurring in a particular organism, such as a C3 and/or C3b binding polypeptide or antigen-binding moiety as described herein. As used herein, the term “heterologous” with respect to a protein domain generally refers to a protein domain that is non-naturally occurring in a particular protein and/or is not joined to or part of the protein when the protein is first identified or generated. In a protein or composition comprising a C3 and/or C3b binding polypeptide and a heterologous polypeptide, the heterologous polypeptide may be any polypeptide that is present as part of a protein or composition in addition to the C3 and/or C3b binding polypeptide.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

K_{D}: As used herein, “K_{D}” refers to an equilibrium dissociation constant, which can be obtained from the ratio of k_{d} to kₐ (i.e., k_{d}/kₐ) and is expressed as a molar concentration (M). K_{D} values can be determined using methods well established in the art, e.g., by using surface plasmon resonance (SPR), e.g., using a Biacore® SPR system.

Linked: As used herein, the term “linked”, when used with respect to two or more moieties, means that the moieties are physically associated or connected with one another. In certain preferred embodiments the linkage is a covalent linkage. In certain embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. A variety of different linker elements that can appropriately be used when engineering polypeptides (e.g., fusion polypeptides) are known in the art (see e.g., Holliger, P., et al. (1993) Proc. Natl. Acad. Sci. USA 90:6444-6448; Poljak, R. J., et al. (1994) Structure 2: 1 121-1123).

Local administration: As used herein, the term “local administration” or “local delivery”, in reference to delivery of a composition or agent, refers to delivery that does not rely upon transport of the composition or agent to its intended target tissue or site via the vascular system. The composition or agent may be delivered directly to its intended target tissue or site, or in the vicinity thereof, e.g., in close proximity to the intended target tissue or site. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site. It will be understood that once having been locally delivered a fraction of a therapeutic agent (typically only a minor fraction of the administered dose) may enter the vascular system and be transported to another location, including back to its intended target tissue or site.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly- refers to a nucleic acid containing 2 to about 10,000, 2 to about 50,000, or 2 to about 100,000 nucleotide monomer units. In some embodiments, the prefix oligo- refers to a nucleic acid containing 2 to about 200 nucleotide monomer units.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Polypeptide: The term “polypeptide”, as used herein, generally has its art-recognized meaning of a polymer of at least three amino acids. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) and immunogenic fragments of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides.

Polypeptides described herein may contain only amino acids such as those that are naturally found in proteins or may include one or more amino acids that are not naturally found in proteins. In some embodiments a polypeptide is composed of standard amino acids, which term refers to the 20 L-amino acids encoded by the standard genetic code. Certain embodiments of the disclosure encompass use of one or more non-standard amino acids in a protein described herein. Such non-standard amino acids may be naturally occurring or may be non-naturally occurring. Useful non-standard amino acids include, e.g., singly and multiply halogenated (e.g., fluorinated) amino acids, D-amino acids, homo-amino acids, N-alkyl amino acids, dehydroamino acids, methoxylated amino acids. One or more amino acids in a polypeptide may be modified, for example, by the addition of a chemical entity such as a carbohydrate group, a phosphate group, a farnesyl group, an isofarnesyl group, a fatty acid group, a protecting group, a moiety comprising a reactive functional group for conjugation, functionalization, or other modification, etc. A polypeptide may be acetylated, e.g., at the N-terminus and/or amidated, e.g., at the C-terminus. In some embodiments one or more non-standard amino acids and/or modifications may be introduced during a chemical synthesis process or by post-translational modification. In certain embodiments, where the present disclosure indicates that X represents any amino acid, the amino acid is a naturally occurring amino acid. In certain embodiments a naturally occurring amino acid (e.g., represented by X) is a standard amino acid. In certain embodiments, where the present disclosure indicates that X represents any naturally occurring amino acid, the amino acid is a standard amino acid. In some embodiments the naturally occurring amino acid is a standard amino acid. Thus, any instance of X may be limited to naturally occurring amino acids or may be limited to standard amino acids unless otherwise stated.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans). In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Systemic: As used herein, the term “systemic” in reference to complement components, refers to complement proteins that are synthesized by liver hepatocytes and enter the bloodstream, or are synthesized by circulating macrophages or monocytes and secreted into the bloodstream.

Systemic administration: As used herein, the term “systemic administration” and like terms are used herein consistently with their usage in the art to refer to administration of an agent such that the agent becomes widely distributed in the body in significant amounts and has a biological effect, e.g., its desired effect, in the blood and/or reaches its desired site of action via the vascular system. Typical systemic routes of administration include administration by (i) introducing the agent directly into the vascular system or (ii) subcutaneous, oral, pulmonary, or intramuscular administration wherein the agent is absorbed, enters the vascular system, and is carried to one or more desired site(s) of action via the blood.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition. The terms “disease”, “disorder”, and “condition” are used interchangeably herein.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The present disclosure is based, in part, on the discovery of polypeptides that surprisingly exhibit binding to C3 and/or C3b with specificity and/or high affinity. In some instances, as compared with a reference polypeptide, a polypeptide (e.g., a monomer, dimer, or a fusion protein) is capable of specifically binding to complement component 3 (C3) and/or C3b with improved binding affinity. In some instances, as compared with a reference polypeptide, a C3 and/or C3b binding polypeptide exhibits increased inhibition of one or both the classical and alternative complement pathways. In some embodiments a reference polypeptide comprises the amino acid sequence of the wild-type Sac7d polypeptide (SEQ ID NO: 1). In some embodiments a reference polypeptide comprises a variant of SEQ ID NO: 1 that has not been selected for binding to C3 and/or C3b. In some embodiments a reference polypeptide comprises a Sac7d family member listed in Table 1. Binding of C3 and/or C3b binding polypeptides disclosed herein to C3 and/or C3b can lead to improved therapeutic properties (e.g., decreased C3 activation) relative to a reference polypeptide. It should be understood that, in general, where a protein, composition, or sequence is described herein as comprising one or more particular sequence(s), element(s), or feature(s), the disclosure provides embodiments that consist, or consist essentially of, such sequence(s), elements, or features unless otherwise indicated or unless the context clearly dictates otherwise.

The present disclosure also provides proteins with multiple targets and/or that bind to different locations on a single target. For example, the disclosure provides fusion proteins, chimeric proteins, and multi-domain proteins that contain multiple binding domains, moieties, or regions for specific targets. In certain instances, a fusion protein is capable of binding with specificity to C3 and/or C3b in addition to vascular endothelial growth factor A (VEGF-A). In certain instances, a fusion protein reduces or inhibits angiogenesis, e.g., as compared with a reference polypeptide. In certain instances, a reference polypeptide comprises an antibody (or fragment thereof) comprising a heavy chain fragment having the amino acid sequence of SEQ ID NO: 36 and a light chain having the amino acid sequence of SEQ ID NO: 35 (ranibizumab (marketed as Lucentis®, Genentech)). Thus, the present disclosure represents a significant advancement in protein therapeutics, e.g., agents that are useful for inhibiting the classical, lectin, and/or alternative complement pathways, such as for treating a complement-associated disorder, and agents that are useful both for inhibiting complement and for inhibiting VEGF, such as for treating a disorder characterized by complement-mediated damage and by aberrant or excessive angiogenesis and/or vascular permeability.

### I. COMPLEMENT SYSTEM

To facilitate understanding of the disclosure, and without intending to limit the invention in any way, this section provides an overview of complement and its pathways of activation. Further details are found, e.g., in Kuby Immunology, 6th ed., 2006; Paul, W. E., Fundamental Immunology, Lippincott Williams & Wilkins; 6th ed., 2008; and Walport MJ., Complement. First of two parts. N Engl J Med., 344(14):1058-66, 2001.

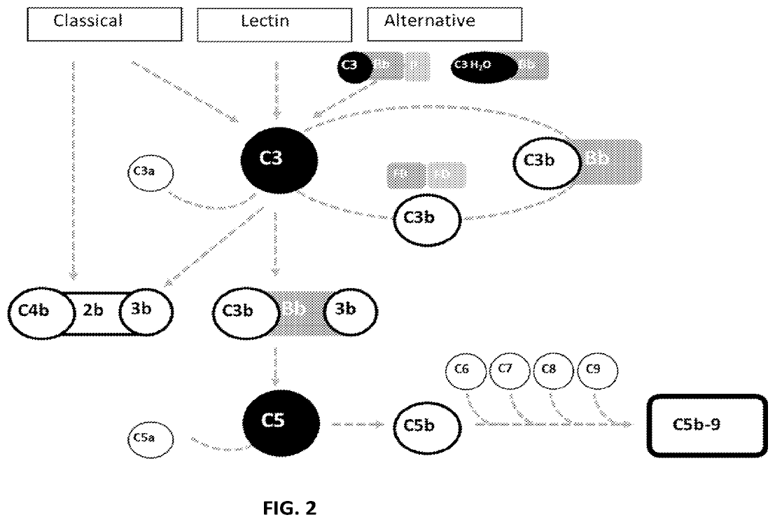

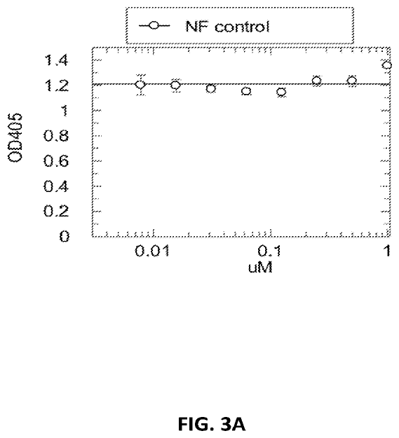

Complement is an arm of the innate immune system that plays an important role in defending the body against infectious agents. The complement system comprises more than 30 serum and cellular proteins that are involved in three major pathways, known as the classical, alternative, and lectin pathways. FIG. 2 provides a schematic overview of these pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylatoxins and mediate multiple reactions in the acute inflammatory response. C3a and C5a are also chemotactic factors that attract immune system cells such as neutrophils.

The alternative pathway is initiated by and amplified at, e.g., microbial surfaces and various complex polysaccharides. In this pathway, hydrolysis of C3 to C3(H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders, e.g., as discussed herein.

### II. C3 AND/OR C3B BINDING POLYPEPTIDES

The disclosure provides C3 and/or C3b binding polypeptides and compositions and methods relating thereto. Polypeptides described herein may be variants of wild-type Sac7d protein that bind to particular targets (e.g., engineered using Nanofitins® technology). In some aspects, the disclosure provides polypeptides derived from the wild-type Sac7d protein or derived from a Sac7d family member that specifically bind to C3 and/or C3b.

Sac7d belongs to a small class of chromosomal proteins discovered in the hyperthermophilic archaeon Sulfolobus acidocaldarius, which are extremely stable in both high temperature and acidity in a natural environment of 85° C. with a pH 2. While the wild-type Sac7d protein binds to double-stranded DNA without any particular sequence preference, Sac7d can be used to obtain polypeptides with specific targets through the introduction of random mutations in residues involved in ligand binding followed by selection of variants with high binding affinity for the specific target. Other proteins homologous to the Sac7d protein (e.g., other members of the Sac7d family of proteins) can also be used to obtain polypeptides that specifically bind to a target of interest (Table 1). A variant of a member of the Sac7d family that binds to a target of interest may be referred to as a Nanofitin®.

TABLE 1
Members of Sac7d family of proteins.

| | | Sequence | |
|---|---|---|---|
| Source | Accession No. | Number | Amino Acid Sequences |
| Sac7d Sulfolobus | WP_011276993.1 | SEQ ID NO: 1 | MVKVKFKYKGEEKEVDTSKIKK |
| acidocaldarius | | | VWRVGKMVSFTYDDNGKTGRGA |
| | | | VSEKDAPKELLDMLARAEREKK |
| Sso7d Sulfolobus | WP_009990119.1 | SEQ ID NO: 2 | MATVKFKYKGEEKEVDISKIKK |
| solfataricus | | | VWRVGKMISFTYDEGGGKTGRG |
| | | | AVSEKDAPKELLQMLEKQKK |
| Sac7e Sulfolobus | WP_011277280.1 | SEQ ID NO: 3 | MAKVRFKYKGEEKEVDTSKIKK |
| acidocaldarius | | | VWRVGKMVSFTYDDNGKTGRGA |
| | | | VSEKDAPKELMDMLARAEKKK |
| Ssh7b Sulfolobus | Uniprot 059632 | SEQ ID NO: 4 | MVTVKFKYKGEEKEVDTSKIKK |
| shibatae | | | VWRVGKMISFTYDEGGGKTGRG |
| | | | AVSEKDAPKELLQMLEKQKK |
| Ssh7a Sulfolobus | WP_009992021.1 | SEQ ID NO: 5 | MATVKFKYKGEEKQVDISKIKK |
| shibatae | | | VWRVGKMISFTYDEGGGKTGRG |
| | | | AVSEKDAPKELLQMLEKQKK |
| DBP7 Sulfolobus | WP_010978621.1 | SEQ ID NO: 6 | MVTVKFKYKGEEKEVDISKIKK |
| tokodaii | | | VWRVGKMISFTYDDNGKTGRGA |
| | | | VSEKDAPKELLQMLEKSGKK |
| Sis7a Sulfolobus | WP_012953380.1 | SEQ ID NO: 7 | MVTVKFKYKGEEKQVDTSKIKK |
| islandicus | | | VWRVGKMISFTYDEGGGKTGRG |
| | | | AVSEKDAPKELLQMLEKQKK |
| Sis7a Sulfolobus | WP_014512200.1 | SEQ ID NO: 8 | MTTVKFKYKGEEKQVDTSKIKK |
| islandicus | | | VWRVGKMISFTYDEGGGKTGRG |
| | | | AVSEKDAPKELLQMLEKQKK |
| Sis7a Sulfolobus | WP_012713334.1 | SEQ ID NO: 9 | MTTVKFKYKGEEKQVDISKIKK |
| islandicus | | | VWRVGKMISFTYDEGGGKTGRG |
| | | | AVSEKDAPKELLQMLEKQKK |
| Mse7 | A4YEA2 | SEQ ID NO: 10 | MATKIKFKYKGQDLEVDISKVK |
| Metallosphaera | | | KVWKVGKMVSFTYDDNGKTGRG |
| sedula | | | AVSEKDAPKELLNMIGKK |
| Mcu7 | F4FYY6 | SEQ ID NO: 11 | MATKIKFKYKGQDLEVDISKVK |
| Metallosphaera | | | KVWKVGKMVSFTYDDNGKTGRG |
| cuprina | | | AVSEKDAPKELLSMIGKK |
| Aho7a Acidianus | f4b991 | SEQ ID NO: 12 | MTTVKFKYKGEEKEVDISKIKK |
| hospitalis | | | VWRVGKMISFTYDDNGKTGRGA |
| | | | VSEKDAPKELLEKLEKK |
| Aho7b Acidianus | f4b9i5 | SEQ ID NO: 13 | MATKVKFKYKGEEKEVDISKIK |
| hospitalis | | | KVWRVGKMISFTYDDNGKTGRG |
| | | | AVSEKDAPKELLDKLEKK |
| Aho7c Acidianus | f4b8×5 | SEQ ID NO: 14 | MATKVKFKYKGEEKEVDISKIK |
| hospitalis | | | KVWRVGKMISFTYDDNGKTGRG |
| | | | AVSEKDAPKELLEKLK |
| Sto7 | q96x56 | SEQ ID NO: 15 | MVTVKFKYKGEEKEVDISKIKK |
| Sulfurisphaera | | | VWRVGKMISFTYDDNGKTGRGA |
| tokodaii | | | VSEKDAPKELLQMLEKSGKK |

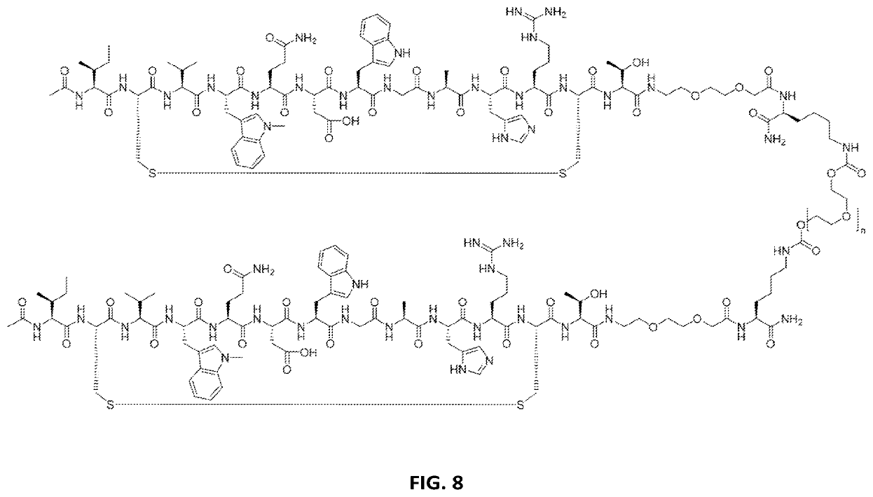

C3 and/or C3b binding polypeptides can be obtained, e.g., by the methods described in PCT Patent Application Publication Nos. WO 2008/068637 and WO 2019/096797, the entirety of each of which is hereby incorporated by reference. As described further in the Examples, in some aspects, C3 and/or C3b binding polypeptides described herein bind to C3 and/or C3b and can also compete with an analog of the C3 inhibitor compstatin (described further below) for binding to C3 and/or C3b. In some embodiments, the ratio of the binding response of a C3 and/or C3b polypeptide described herein to C3 and/or C3b relative to a compstatin analog described herein (e.g., the compound of FIG. 8) in a competition assay (e.g., an ELISA competition assay) is about 2, about 3, about 4, about 5, about 6, about 7, about 8, about 9, about 10, about 11, about 12, about 13, about 14, about 15, about 20, about 25, about 30, about 35, about 40, or higher. As described further in the Examples, in some aspects, C3 and/or C3b binding polypeptides described herein potently inhibit complement activation.

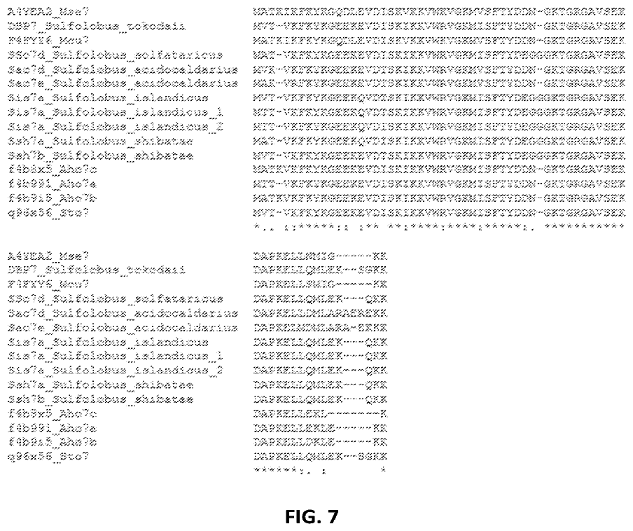

C3 and/or C3b binding polypeptides may be described with reference to a consensus amino acid sequence for the Sac7d family of proteins, which may be generated based on members of the family (see, e.g., Table 1 and FIG. 7). An exemplary consensus amino acid sequence is MXXXVXFKYKGEEKXVDXSKIKKVWRVGKMXSFTYDXXXGKTGRGAVSEKDAPKEL XXXLXXXXXXXX (SEQ ID NO: 16), where the amino acids at positions 2, 3, 4, 6, 15, 18, 31, 37-39, 57, 58, 59, and 61-68 are as follows (Table 2):

TABLE 2

| Position | Amino Acid |
|---|---|
| (2) . . . (2) | X is V, A or T |
| (3) . . . (3) | X is T or K |
| (4) . . . (4) | X is - or K |
| (6) . . . (6) | X is R or K |
| (15) . . . (15) | X is E or Q |
| (18) . . . (18) | X is T or I |
| (31) . . . (31) | X is V or I |
| (37) . . . (39) | X X X is EGG or DN- |
| (57) . . . (57) | X is M or L |
| (58) . . . (58) | X is Q, D or E |
| (59) . . . (59) | X is M or K |
| (61) . . . (68) | XXXXXXXX is -------K, EKS--GKK |
| | (SEQ ID NO: 82), EK---QKK (SEQ |
| | ID NO: 83), ARAEREKK (SEQ ID |
| | NO: 79), ARAERE-K (SEQ ID NO: |
| | 81), ARAERE-- (SEQ ID NO: 80), |
| | ARA-EKKK (SEQ ID NO: 84), E--- |
| | --KK, EKS--GKK (SEQ ID NO: 82), |
| | ACAEREKK (SEQ ID NO: 85) or |
| | ACA-EKKK (SEQ ID NO: 86) |

In some embodiments, the consensus sequence can be varied to include one or more amino acid substitutions that may improve one or more properties of a polypeptide that is otherwise within the scope of the consensus sequence, as compared to a polypeptide having the same sequence but without such substitutions(s), without detrimentally affecting binding of such a polypeptide to a target. For example, the amino acid at position 17 (which is D in the consensus sequence SEQ ID NO: 16) may be changed to E and/or the amino acid at position 38 (which is G or N in the consensus sequence of SEQ ID NO: 16), may be changed to Q and/or the amino acid at position 59 (which is M or K in the consensus sequence of SEQ ID NO: 16) may be changed to L. A variant of the consensus amino acid sequence of SEQ ID NO: 16 that incorporates these possible substitutions is as follows: MXXXVXFKYKGEEKXVXXSKIKKVWRVGKMXSFTYDXXXGKTGRGAVSEKDAPKEL XXXLXXXXXXXX (SEQ ID NO:76), where the amino acids at positions 2, 3, 4, 6, 15, 17, 18, 31, 37-39, 57, 58, 59, and 61-68 are as follows (Table 3):

TABLE 3

| Position | Amino Acid |
|---|---|
| (2) . . . (2) | X is V, A or T |
| (3) . . . (3) | X is T or K |
| (4) . . . (4) | X is - or K |
| (6) . . . (6) | X is R or K |
| (15) . . . (15) | X is E or Q |
| (17) . . . (17) | X is D or E |
| (18) . . . (18) | X is T or I |
| (31) . . . (31) | X is V or I |
| (37) . . . (39) | X X X is EGG, DN- or DQ- |
| (57) . . . (57) | X is M or L |
| (58) . . . (58) | X is Q, D or E |
| (59) . . . (59) | X is M, K, or L |
| (61) . . . (68) | XXXXXXXX is -------K, EKS--GKK |
| | (SEQ ID NO: 82), EK---QKK (SEQ |
| | ID NO: 83), ARAEREKK (SEQ ID |
| | NO: 79), ARAERE-K (SEQ ID NO: |
| | 81), ARAERE--(SEQ ID NO: 80), |
| | ARA-EKKK (SEQ ID NO: 84), E--- |
| | --KK, EKS--GKK (SEQ ID NO: 82), |
| | ACAEREKK (SEQ ID NO: 85) or |
| | ACA-EKKK (SEQ ID NO: 86) |

Mutations may be introduced into a Sac7d family consensus sequence (e.g., SEQ ID NO: 16 or SEQ ID NO: 76) at selected positions to generate a consensus sequence for C3 and/or C3b binding polypeptides. In some embodiments, a C3 and/or C3b binding polypeptide has a consensus sequence that comprises a variant of a Sac7d consensus amino acid sequence described herein (e.g., SEQ ID NO: 16 or SEQ ID NO: 76), wherein the variant has between 4 and 22 mutations as compared to the Sac7d family consensus sequence of SEQ ID NO: 16 or SEQ ID NO: 76. In some embodiments the variant consensus sequence has between 4 and 8, between 4 and 12, between 4 and 16, between 6 and 10, between 6 and 12 mutations as compared to the Sac7d family consensus sequence of SEQ ID NO: 16 or SEQ ID NO: 76. In some embodiments the mutations are at positions selected from positions 2, 3, 6, 8, 9, 10, 11, 12, 14, 15, 17, 18, 22, 23, 25, 27, 28, 29, 30, 32, 34, 35, 37, 38, 40, 41, 42, 44, 46, 48, 49, 50, 51, 52, and 53 in SEQ ID NO: 16 or in SEQ ID NO: 76, which correspond to V2, K3, K5, K7, Y8, K9, G10, E11, K13, E14, D16, T17, K21, K22, W24, V26, G27, K28, M29, S31, T33, Y34, D36, N37, G38, K39, T40, R42, A44, S46, E47, K48, D49, A50 and P51 of Sac7d (SEQ ID NO: 1). In some embodiments, a C3 and/or C3b binding polypeptide comprises mutations at positions 8, 9, 10, 22, 23, 25, 27, 30, 32, 34, 42, 44, 46, and/or 48 of a polypeptide defined by a Sac7d family consensus sequence described herein (e.g., SEQ ID NO: 16 or SEQ ID NO: 76), wherein the amino acids at positions 8, 9, 10, 22, 23, 25, 27, 30, 32, 34, 42, 44, 46, and/or 48 are selected such that the polypeptide binds to C3 and/or C3b with increased affinity relative to a reference polypeptide (e.g., Sac7d). Thus, in some embodiments, a C3 and/or C3b binding polypeptide comprises the amino acid sequence MXXXVXFXXXGEEKXVXXSKIXXVXRXGKXXXFXYDXXXGKXGXGXVXEKDAPKEL XXXLXXXXXXXX (SEQ ID NO:77), wherein each X at positions 8, 9, 10, 22, 23, 25, 27, 30, 32, 34, 42, 44, 46, and 48 independently represents any amino acid and the polypeptide has higher affinity for C3 and/or C3b than a reference polypeptide (e.g., Sac7d) and wherein the amino acids at positions 2, 3, 4, 6, 15, 17, 18, 31, 37 to 39, 57, 58, 59, and 61 to 68 are as set forth in Table 3.

Variants of Sac7d within the scope of SEQ ID NO: 77 that bind to C3 were identified as described in the Examples (e.g., SEQ ID NO: 27, SEQ ID NO: 33, SEQ ID NO: 34, which are described below) and are representative of a family of C3 and/or C3b binding polypeptides that inhibit complement activation. These polypeptides have mutations at positions corresponding to positions 8, 9, 10, 22, 23, 25, 27, 30, 32, 34, 42, 44, 46, and/or 48 in SEQ ID NO 77 (indicated in bold and underlined in SEQ ID NO:77 and certain other sequences below). Thus, in some embodiments, the amino acid sequence of a C3 and/or C3b binding polypeptide comprises or consists of the amino acid sequence MXXXVXFXXXGEEKXVXXSKIXXVXRXGKXXXFXYDXXXGKXGXGXVXEKDAPKEL XXXLXXXXXXXX (SEQ ID NO:78) or a C3 and/or C3b binding portion thereof, wherein the amino acids at positions 2, 3, 4, 6, 15, 17, 18, 31, 37 to 39, 57, 58, 59, and 61 to 68 are as set forth in Table 3, and wherein the polypeptide comprises at least 2, 3, 4, 5, 6, 7, 8, 9, 10, 11, 12, 13, or 14 of the following: E, A, or D at position 8; A at position 9; T at position 10; Y, A, or S at position 22; Q, N, or A at position 23; Y at position 25; S, V, or T at position 27; E or D at position 30; A, I, L, V, Y, M, F or H at position 32; A, T or S at position 34; T or I at position 42; W at position 44; A, I, L, V, Y, M, F or H at position 46; and S at position 48.

Certain C3 and/or C3b binding polypeptides described herein comprise a methionine at the N-terminus. However, such methionine is not necessary for C3 and/or C3b binding and complement inhibiting activity of the polypeptides and may be omitted from any of the polypeptides described herein. It is further contemplated that the amino acid corresponding to position 2 of SEQ ID NO: 78 or any Sac7d family member may be omitted in certain embodiments. Similarly, one or more amino acids located at the C-terminus of the polypeptides may be omitted. For example, in certain embodiments one or more of the amino acids located after residue L58 of a variant of Sac7d (SEQ ID NO:1) or located after L60 of a variant of SEQ ID NO: 16, SEQ ID NO: 76, or SEQ ID NO: 78 or located after L59 of a variant of Sso7d (SEQ ID NO: 2) or located after L56 of a variant of Aho7c (SEQ NO: 14) or located after corresponding amino acids in other Sac7d family members may be omitted in certain embodiments. Accordingly, in some embodiments a C3 and/or C3b binding polypeptide comprises amino acids 3-60 of SEQ ID NO: 78. Where the present disclosure refers to a C3 and/or C3b binding portion of SEQ ID NO 78, the disclosure provides embodiments in which such portion may comprise or consist of amino acids 3-60, 3-61, 3-62, 3-63, 3-64, 3-65, 3-66, 3-67, 3-68, 2-60, 2-61, 2-62, 2-63, 2-64, 2-65, 2-66, 2-67, 2-68, 1-60, 1-61, 1-62, 1-63, 1-64, 1-65, 1-66, or 1-67 of SEQ ID NO: 78. In some aspects, one or more amino acids may be added at the C-terminal end of any polypeptide sequence (e.g., any C3 and/or C3b binding sequence or C3 and/or C3b binding portion thereof) described herein. For example, in certain embodiments the C-terminal end of any polypeptide sequence disclosed herein (e.g., any C3 and/or C3b binding sequence or portion thereof) may be extended by a sequence comprising or consisting of one or more (e.g., 1, 2, 3, or more) lysine residues. Certain Nanofitins selected for binding to C3 and/or C3b comprise the amino acid sequence of SEQ ID NO: 78 or a C3 and/or C3b binding portion thereof (e.g., amino acids 2-60), wherein the amino acids at positions 25 and 44 in SEQ ID NO: 78 (corresponding to positions 24 and 42 in the sequence of Sac7d (SEQ ID NO: 1)) are Y and W, respectively. Without wishing to be bound by any theory, it is believed that the substitutions Y at position 25 and W at position 44 (corresponding to W24Y and R42W in Sac7d (SEQ ID NO: 1)) play a particularly important role in the C3 and C3b binding activity and complement inhibiting activity of this family of polypeptides. In some embodiments, the amino acid sequence of a C3 and/or C3b binding polypeptide comprises the amino acid sequence: MXXXVXFXXXGEEKXVXXSKIXXVYRXGKXXXFXYDXXXGKXGWGXVXEKDAPKE LXXXLXXXXXXXX (SEQ ID NO: 17) or a C3 and/or C3b binding portion thereof (e.g., amino acids 2-60), wherein the amino acids at positions 2, 3, 4, 6, 15, 17, 18, 31, 37 to 39, 57, 58, 59, and 61 to 68 are as listed in Table 3, and wherein the amino acids at positions 8, 9, 10, 22, 23, 27, 30, 32, 34, 42, 46, and 48 are as follows (Table 4):

TABLE 4

| Position | Amino Acid |
|---|---|
| (8) . . . (10) | X can be any amino acid |
| (22) . . . (23) | X can be any amino acid |
| (27) . . . (27) | X can be any amino acid |
| (30) . . . (30) | X can be any amino acid |
| (32) . . . (32) | X is A, I, L, V, Y, M, F or H |
| (34) . . . (34) | X is S, T or A |
| (42) . . . (42) | X is T or I |
| (46) . . . (46) | X is A, I, L, V, Y, M, F or H |
| (48) . . . (48) | X can be any amino acid |

In certain embodiments the amino acids at positions 2, 3, 4, 6, 15, 17, 18, 31, 37 to 39, 57, 58, 59, and 61 to 68 in SEQ ID NO: 17 are as listed in Table 3, and the amino acids at positions 8, 9, 10, 22, 23, 27, 30, 32, 34, 42, 46, and 48 in SEQ ID NO: 17 are as follows (Table 5):

TABLE 5

| | | Preferred amino acids |
|---|---|---|
| Position | Amino acid | in certain embodiments |
| 8 | X can be any amino acid | D, E A, V, or P |
| 9 | X can be any amino acid | A, L, I, V, T or H |
| 10 | X can be any amino acid | T, I |
| 22 | X is A, S, T, V, I, L, Y, or F | F, Y, A, L, or S |
| 23 | X can be any amino acid | H, N, A, K, Q |
| 27 | X is S, T, V, I, L, Y, or F | I, L, Y, V or T |
| 30 | X can be any amino acid | D, E, S, or A |
| 32 | X is L, I, V, Y, F, M, or H | L, Y, I or M |
| 34 | X is S, T or A | S, T or A |
| 42 | X is T or I | T or I |
| 46 | X is F, Y, I, V, L, M or H | Y, F |
| 48 | X is S, T, H or R | S |

In certain embodiments C3 and/or C3b binding polypeptides have T at position 10, L at position 32, and Y at position 46 in SEQ ID NO: 77 in addition to Y at position 25 and W at position 44 in SEQ ID NO: 77. Thus, in some embodiments, a C3 and/or C3b binding polypeptide comprises the amino acid sequence: MXXXVXFXXTGEEKXVXXSKIXXVYRXGKXXLFXYDXXXGKXGWGYVXEKDAPKE LXXXLXXXXXXXX (SEQ ID NO: 18) or a C3 and/or C3b binding portion thereof (e.g., amino acids 2-60), wherein the amino acids at positions 2, 3, 4, 6, 15, 17, 18, 31, 37 to 39, 57, 58, 59, and 61 to 68 are as listed in Table 3, and wherein the amino acids at positions 8, 9, 22, 23, 27, 30, 34, 42, and 48 are as follows (Table 6):

TABLE 6

| Position | Amino Acid |
|---|---|
| (8) . . . (9) | X can be any amino acid |
| (22) . . . (23) | X can be any amino acid |
| (27) . . . (27) | X can be any amino acid |
| (30) . . . (30) | X can be any amino acid |
| (34) . . . (34) | X is S, T or A |
| (42) . . . (42) | X is T or I |
| (48) . . . (48) | X can be any amino acid |

In certain embodiments the amino acids at positions 2, 3, 4, 6, 15, 17, 18, 31, 37 to 39, 57, 58, 59, and 61 to 68 in SEQ ID NO: 18 are as listed in Table 3, and the amino acids at positions 8, 9, 22, 23, 27, 30, 34, 42, and 48 in SEQ ID NO: 18 are as follows (Table 7):

TABLE 7

| | | Preferred amino acids |
|---|---|---|
| Position | Amino acid | in certain embodiments |
| 8 | X can be any amino acid | D, E A, V, or P |
| 9 | X can be any amino acid | A, L, I, V, T or H |
| 22 | X is A, S, T, V, I, L, Y, or F | F, Y, A, L, or S |
| 23 | X can be any amino acid | H, N, A, K, Q |
| 27 | X is S, T, V, I, L, Y, or F | I, L, Y, V or T |
| 30 | X can be any amino acid | D, E, S, or A |
| 34 | X is S, T or A | S, T or A |
| 42 | X is T or I | T or I |
| 48 | X is S, T, H or R | S |

In certain embodiments C3 and/or C3b binding polypeptides have A at position 9, D at position 30, S at position 34, I at position 42, and S at position 48 in SEQ ID NO: 77, in addition to Y at position 25, W at position 44, T at position 10, L at position 32, and Y at position 46 in SEQ ID NO: 77. Thus, in some embodiments, a C3 and/or C3b binding polypeptide comprises the amino acid sequence of MXXXVXFXATGEEKXVXXSKIXXVYRXGKDXLFSYDXXXGKIGWGYVSEKDAPKEL XXXLXXXXXXXX (SEQ ID NO: 19) or a C3 and/or C3b binding portion thereof (e.g., amino acids 2-60), wherein the amino acids at positions 2, 3, 4, 6, 15, 17, 18, 31, 37 to 39, 57, 58, 59, and 61 to 68 are as listed in Table 3, and wherein the amino acids at positions 8, 22, 23, and 27 can be any amino acid.

In certain embodiments the amino acids at positions 2, 3, 4, 6, 15, 17, 18, 31, 37 to 39, 57, 58, 59, and 61 to 68 SEQ ID NO: 19 are as listed in Table 3 and the amino acids at positions 8, 22, 23, and 27 in SEQ ID NO: 19 are as follows (Table 8):

TABLE 8

| | | Preferred amino acids |
|---|---|---|
| Position | Amino acid | in certain embodiments |
| 8 | X can be any amino acid | D, E A, V, or P |
| 22 | X is A, S, T, V, I, L, Y, or F | F, Y, A, L, or S |
| 23 | X can be any amino acid | H, N, A, K, Q |
| 27 | X is S, T, V, I, L, Y, or F | I, L, Y, V or T |

Certain C3 and/or C3b binding polypeptides comprise the amino acid sequence of SEQ ID NO: 77 or a C3 and/or C3b binding portion thereof (e.g., amino acids 2-60), wherein the sequence has D at position 8, S at position 22, A at position 23, and T at position 27 in SEQ ID NO 77 in addition to Y at position 25, W at position 44, T position 10, L at position 32, Y at position 46, A at position 9, D at position 30, S at position 34, I at position 42, and S at position 48. Thus, in some embodiments, a C3 and/or C3b binding polypeptide comprises the amino acid sequence: MXXXVXFDATGEEKXVXXSKISAVYRTGKDXLFSYDXXXGKIGWGYVSEKDAPKELX XXLXXXXXXXX (SEQ ID NO: 20) or a C3 and/or C3b binding portion thereof (e.g., amino acids 2-60), wherein the amino acids at positions 2, 3, 4, 6, 15, 17, 18, 31, 37 to 39, 57, 58, 59, and 61 to 68 are as listed in Table 3.

Certain C3 and/or C3b binding polypeptides comprise the amino acid sequence of SEQ ID NO: 77 or a C3 and/or C3b binding portion thereof (e.g., amino acids 2-60), wherein the sequence has A at position 8, A at position 22, N at position 23, and V at position 27 in SEQ ID NO: 77 in addition to Y at position 25, W at position 44, T at position 10, L at position 32, Y at position 46, A at position 9, D at position 30, S at position 34, I at position 42, and S at the position 48 in SEQ ID NO: 77 Thus, in some embodiments, a C3 and/or C3b binding polypeptide comprises the amino acid sequence MXXXVXFAATGEEKXVXXSKIANVYRVGKDXLFSYDXXXGKIGWGYVSEKDAPKEL XXXLXXXXXXXX (SEQ ID NO: 21) or a C3 and/or C3b binding portion thereof (e.g., amino acids 2-60), wherein the amino acids at positions 2, 3, 4, 6, 15, 17, 18, 31, 37 to 39, 57, 58, 59, and 61 to 68 are as listed in Table 3.

Where the present disclosure refers to a C3 and/or C3b binding portion of SEQ ID NO: 17, SEQ ID NO: 18, SEQ ID NO: 19, SEQ ID NO: 20, or SEQ ID NO: 21, the disclosure provides embodiments in which such portion may comprise or consist of amino acids 2-60, 2-61, 2-62, 2-63, 2-64, 2-65, 2-66, 2-67, 2-68, 1-60, 1-61, 1-62, 1-63, 1-64, 1-65, 1-66, or 1-67 of the respective sequence. In some embodiments such portion may comprise or consist of amino acids 3-60, 3-61, 3-62, 3-63, 3-64, 3-65, 3-66, 3-67, or 2-68 of the respective sequence.

In some embodiments, C3 and/or C3 binding polypeptides are variants of Sac7d (SEQ ID NO: 1). In certain embodiments a variant has between 4 and 22 substitutions as compared to Sac7d. A variant of Sac7d (SEQ ID NO: 1) may be represented as MVKVKFXXXGEEKEVXTSKIXXVXRXGKXVXFXYDDXGKXGXGXVXEKDAPKELLD XLARAEREKK (SEQ ID NO: 66), wherein each X independently represents any amino acid, wherein certain positions (e.g., positions 7, 8, 9, 21, 22, 24, 26, 29, 31, 33, 40, 42, 44, and/or 46) of the sequence may be varied for C3 and/or C3b binding, and wherein the polypeptide comprises: D or E at position 16, N or Q at position 37, and L or M at position 57. Positions 7, 8, 9, 21, 22, 24, 26, 29, 31, 33, 40, 42, 44, and 46 are indicated in bold and are underlined in SEQ ID NO: 66 and certain other sequences below. In certain embodiments the C3 and/or C3b binding polypeptide comprises at least 2, 3, 4, 5, 6, 7, 8, 9, 10, 11, 12, 13, or 14 of the following: E, A, or D at position 7; A at position 8; T at position 9; Y, A, or S at position 21; Q, N, or A at position 22; Y at position 24; S, V, or T at position 26; E or D at position 29; A, I, L, V, Y, M, F or H at position 31; A, T or S at position 33; T or I at position 40; W at position 42; A, I, L, V, Y, M, F or H at position 44; and S at position 46 in SEQ ID NO:66. As noted above, the methionine at the N-terminus is not necessary for C3 and/or C3b binding and complement inhibiting activity of the polypeptides and may be omitted from any of the polypeptides described herein, and it is also contemplated that the amino acid at position 2 may be omitted or substituted (e.g., with an A as in certain other Sac7d family members). Similarly, one or more amino acids located at the C-terminus of the polypeptides are not necessary for C3 and/or C3b binding and complement inhibiting activity of the polypeptides and may be omitted. For example, in certain embodiments one or more of the amino acids located after residue L58 of SEQ ID NO: 66 may be omitted. Where the present disclosure refers to a C3 and/or C3b binding portion of SEQ ID NO: 66 (or of any sequence in which one or more amino acids indicated by an X in SEQ ID NO: 66 is further specified), such portion may comprise or consist of amino acids 2-58, 2-59, 2-60, 2-61, 2-62, 2-63, 2-64, 2-65, 2-66, 1-58, 1-59, 1-60, 1-61, 1-62, 1-63, 1-64, or 1-65 of SEQ ID NO: 66 (or of the respective sequence). In some embodiments such portion may comprise or consist of amino acids 3-58, 3-59, 3-60, 3-61, 3-62, 3-63, 3-64, or 3-65, or 2-66, of SEQ ID NO: 66 (or of the respective sequence).

In certain Nanofitins selected for binding to C3 and C3b, the amino acids at positions 24 and 42 in SEQ ID NO: 66 are Y and W, respectively. Thus, in some embodiments, a C3 and/or C3b binding polypeptide comprises the amino acid sequence MVKVKFXXXGEEKEVXTSKIXXVYRXGKXVXFXYDDXGKXGWGXVXEKDAPKELLD XLARAERE (SEQ ID NO: 22) or a C3 and/or C3b binding portion thereof (e.g., amino acids 2-58), where the amino acids at positions 7, 8, 9, 16, 21, 22, 26, 29, 31, 33, 37, 40, 44, 46, and 57 are as set forth in the following table (Table 9):

TABLE 9

| Position | Amino Acid |
|---|---|
| (7) . . . (9) | X can be any amino acid |
| (16) . . . (16) | X is E or D |
| (21) . . . (22) | X can be any amino acid |
| (26) . . . (26) | X can be any amino acid |
| (29) . . . (29) | X can be any amino acid |
| (31) . . . (31) | X is A, I, L, V, Y, M, F or H |
| (33) . . . (33) | X is S, T or A |
| (37) | X is N or Q |
| (40) . . . (40) | X is T or I |
| (44) . . . (44) | X is A, I, L, V, Y, M, F or H |
| (46) . . . (46) | X can be any amino acid |
| 57 | X is M or L |

In certain embodiments the amino acids at positions 16, 37, and 57 of SEQ ID NO: 22 are as set forth in Table 9, and the amino acids at positions 7, 8, 9, 21, 22, 26, 29, 31, 33, 40, 44, and 46 of SEQ ID NO: 22 are as follows (Table 10):

TABLE 10

| | | Preferred amino acid |
|---|---|---|
| Position | Amino acid | in certain embodiments |
| 7 | X can be any amino acid | D, E A, V, or P |
| 8 | X can be any amino acid | A, L, I, V, T or H |
| 9 | X can be any amino acid | T, I |
| 21 | X is A, S, T, V, I, L, Y, or F | F, Y, A, L, or S |
| 22 | X can be any amino acid | H, N, A, K, Q |
| 26 | X is S, T, V, I, L, Y, or F | I, L, Y, V or T |
| 29 | X can be any amino acid | D, E, S, or A |
| 31 | X is L, I, V, Y, F, M, or H | L, Y, I or M |
| 33 | X is S, T or A | S, T or A |
| 40 | X is T or I | T or I |
| 44 | X is F, Y, I, V, L, M or H | Y, F |
| 46 | X is S, T, H or R | S |

In some embodiments, a C3 and/or C3b binding polypeptide comprises an amino acid sequence of SEQ ID NO: 22 or a C3 and/or C3b binding portion thereof (e.g., amino acids 2-58), wherein the amino acid sequence comprises D or A at position 7, A at position 8, T at position 9, S or A at position 21, A or N at position 22, Y at position 24, T or V at position 26, D at position 29, L at position 31, S at position 33, I at position 40, W at position 42, Y at position 44, and S at position 46, and the amino acids at positions 16, 37, and 57 are as set forth in Table 9.

Certain Nanofitins selected for binding to C3 and C3b have T at position 9, L at position 31, and Y at position 44 in SEQ ID NO: 65 in addition to Y at position 24 and W at position 42. Thus, in some embodiments, a C3 and/or C3b binding polypeptide comprises the amino acid sequence: MVKVKFXXTGEEKEVXTSKIXXVYRXGKXVLFXYDDXGKXGWGYVXEKDAPKELLD XLARAERE (SEQ ID NO: 23) or a C3 and/or C3b binding portion thereof (e.g., amino acids 2-58), where the amino acids at positions 7, 8, 16, 21 22, 26, 29, 33, 37, 40, 46, and 57 are as set forth in Table 9.

In certain embodiments the amino acids at positions 16, 37, and 57 in SEQ ID NO: 23 are as set forth in Table 9 and the amino acids at positions 7, 8, 21, 22, 26, 29, 33, 40, and 46 in SEQ ID NO: 23, are as set forth in the following table (Table 11):

TABLE 11

| | | Preferred amino acid |
|---|---|---|
| Position | Amino acid | in certain embodiments |
| 7 | X can be any amino acid | D, E A, V, or P |
| 8 | X can be any amino acid | A, L, I, V, T or H |
| 21 | X is A, S, T, V, I, L, Y, or F | F, Y, A, L, or S |
| 22 | X can be any amino acid | H, N, A, K, Q |
| 26 | X is S, T, V, I, L, Y, or F | I, L, Y, V or T |
| 29 | X can be any amino acid | D, E, S, or A |
| 33 | X is S, T or A | S, T or A |
| 40 | X is T or I | T or I |
| 46 | X is S, T, H or R | S |

In some embodiments, a C3 and/or C3b binding polypeptide comprises an amino acid sequence of SEQ ID NO: 23, wherein the amino acid sequence comprises D or A at position 7, A at position 8, S or A at position 21, A or N at position 22, T or V at position 26, D at position 29, S at position 33, I at position 40, and S at position 46, and the amino acids at positions 16, 37, and 57 are as set forth in Table 9.

Certain C3 and/or C3b binding Nanofitins have A at position 8, D at position 29, S at position 33, I at position 40, and S at position 46 in SEQ ID NO: 65 in addition to Y at position 24, W at position 42, T at position 9, L at position 31, and Y at position 44. Thus, in certain embodiments a C3 and/or C3b binding polypeptide comprises the amino acid sequence MVKVKFXATGEEKEVXTSKIXXVYRXGKDVLFSYDDXGKIGWGYVSEKDAPKELLDX LARAERE (SEQ ID NO: 24) or a C3 and/or C3b binding portion thereof (e.g., amino acids 2-58), wherein the amino acids at positions 7, 16, 21, 22, 26, 37 and 57 are as set forth in Table 9.

In certain embodiments the amino acids at positions 7, 21, 22, and 26 in SEQ ID NO: 24, are as set forth in the following table (Table 12), and the amino acids at positions 16, 37, and 57 are as set forth in Table 9.

TABLE 12

| | | Preferred amino acid |
|---|---|---|
| Position | Amino acid | in certain embodiments |
| 7 | X can be any amino acid | D, E A, V, or P |
| 21 | X is A, S, T, V, I, L, Y, or F | F, Y, A, L, or S |
| 22 | X can be any amino acid | H, N, A, K, Q |
| 26 | X is S, T, V, I, L, Y, or F | I, L, Y, V or T |

In some embodiments, a C3 and/or C3b binding polypeptide comprises an amino acid sequence of SEQ ID NO: 24 or a C3 and/or C3b binding portion thereof (e.g., amino acids 2-58), wherein the amino acid sequence comprises D or A at position 7, S or A at position 21, A or N at position 22, and T or V at position 26, and the amino acids at positions 16, 37, and 57 are as set forth in Table 9. In some embodiments a C3 and/or C3b binding polypeptide comprises an amino acid sequence of SEQ ID NO: 24 or a C3 and/or C3b binding portion thereof (e.g., amino acids 2-58), wherein the amino acid sequence comprises D at position 7, S at position 21, A at position 22, and T at position 26, and the amino acids at positions 16, 37, and 57 are as set forth in Table 9. In some embodiments a C3 and/or C3b binding polypeptide comprises an amino acid sequence of SEQ ID NO: 24 or a C3 and/or C3b binding portion thereof (e.g., amino acids 2-58), wherein the amino acid sequence comprises A at position 7, A at position 21, N at position 22, and V at position 26, and the amino acids at positions 16, 37, and 57 are as set forth in Table 9. In each sequence in which positions 16, 37, and 57 may vary, embodiments are disclosed in which the amino acids are as set forth in any row of Table 13. In certain embodiments a C3 and/or C3b binding polypeptide comprises SEQ ID NO: 27, SEQ ID NO: 33, or SEQ ID NO: 34 (see Table 16) or a C3 and/or C3b binding portion of SEQ ID NO: 27, SEQ ID NO: 33, or SEQ ID NO: 34.

TABLE 13

| Position 16 | Position 37 | Position 57 |
|---|---|---|
| D | N | M |
| D | N | L |
| D | Q | M |
| D | Q | L |
| E | N | M |
| E | N | L |
| E | Q | M |
| E | Q | L |

In some embodiments, a C3 and/or C3b binding polypeptide is a variant of a Sac7d family member other than Sac7d, e.g., Sac7e from Sulfolobus acidocaldarius, Sso7d from Sulfolobus solfataricus, Ssh7b from Sulfolobus shibatae, Ssh7a from Sulfolobus shibatae, DBP7 from Sulfolobus tokodaii, Sis7a from Sulfolobus islandicus, Mse7 from Metallosphaera sedula, Mcu7 from Metallosphaera cuprina, Aho7a from Acidianus hospitalis, Aho7b from Acidianus hospitalis, Aho7c from Acidianus hospitalis or Sto7 from Sulfurisphaera tokodaii. Sequences of these Sac7d family members are set forth in Table 1, and an alignment of the sequences is depicted in FIG. 7. The sequences of C3 and/or C3b binding polypeptides that are variants of these Sac7d family members can be obtained by substituting the amino acids located at positions 7, 8, 9, 21, 22, 24, 26, 29, 31. 33, 40. 42, 44, and 46 in C3 and/or C3b binding variants of Sac7d described herein (SEQ ID Nos: 22, 23, 24, 27, 33, or 34, with X as described in Tables 9, 10, 11, and 12 above) into the corresponding positions of the respective Sac7d family member (i.e., the positions aligning with positions 7, 8, 9, 21, 22, 24, 26, 29, 31. 33, 40. 42, 44, and 46 of Sac7d in the alignment shown in FIG. 7). Further, in the case of those Sac7d family members that have D at the position corresponding to position 16 of Sac7d, such D may be substituted by E. In the case of those Sac7d family members that have N at the position corresponding to position 37 of Sac7d, such N may be substituted by Q. In the case of those Sac7d family members that have M at the position corresponding to position 57 in Sac7d, such M may be substituted by L.

For example, positions 7, 8, 9, 21, 22, 24, 26, 29, 31, 33, 40, 42, 44, and 46 in Sac7d correspond to positions 7, 8, 9, 21, 22, 24, 26, 29, 31, 33, 41, 43, 45, and 47 in Sso7d. Thus, in some embodiments a C3 and/or C3b binding polypeptide that is a variant of Sso7d may have at least 2, 3, 4, 5, 6, 7, 8, 9, 10, 11, 12, 13, or 14 of the following substitutions in SEQ ID NO:2: E, A, or D at position 7; A at position 8; T at position 9; Y, A, or S at position 21; Q, N, or A at position 22; Y at position 24; S, V, or T at position 26; E or D at position 29; A, I, L, V, Y, M, F or H at position 31; A, T or S at position 33; T or I at position 41; W at position 43; A, I, L, V, Y, M, F or H at position 45; and S at position 47. Positions 16 and 58 in Sso7d correspond to (align with) positions 16 and 57 in Sac7d. Ssd7d has D at position 16 and M at position 58. Thus, any variant of Sso7d may comprise D or E at position 16 (corresponding to position 16 in Sac7d) and/or may comprise L or M at position 58 (corresponding to position 57 in Sac7d). In some embodiments the amino acids at positions 16 and 58 of an Sso7d variant are D and L, respectively. In some embodiments the amino acids at positions 16 and 58 of an Sso7d variant are E and L respectively. In some embodiments the amino acids at positions 16 and 58 of an Sso7d variant are D and M, respectively. In some embodiments the amino acids at positions 16 and 58 of an Sso7d variant are E and M, respectively.

In certain embodiments a C3 and/or C3b binding polypeptide that is a variant of Sso7d has Y at position 24 and W at position 43 (corresponding to a Sac7d variant of SEQ ID NO: 22 having Y at position 24 and W at position 42) and comprises the amino acid sequence MATVKFXXXGEEKEVXISKIXXVYRXGKXIXFXYDEGGGKXGWGXVXEKDAPKELLQ XLEKQKK (SEQ ID NO: 25) or a C3 and/or C3b binding portion thereof (e.g., amino acids 2-59), wherein the amino acids at positions 7, 8, 9, 16, 21, 22, 26, 29, 31, 33, 41, 45, 47, and 58 are as set forth in the following table (Table 14):

TABLE 14

| Position | Amino Acid |
|---|---|
| (7) . . . (9) | X can be any amino acid |
| (16) . . . (16) | X is E or D |
| (21) . . . (22) | X can be any amino acid |
| (26) . . . (26) | X can be any amino acid |
| (29) . . . (29) | X can be any amino acid |
| (31) . . . (31) | X is A, I, L, V, Y, M, F or H |
| (33) . . . (33) | X is S, T or A |
| (41) . . . (41) | X is T or I |
| (45) . . . (45) | X is A, I, L, V, Y, M, F or H |
| (47) . . . (47) | X can be any amino acid |
| 58 | X is M or L |

In certain embodiments the amino acids at positions 7, 8, 9, 21, 22, 26, 29, 31, 33, 41, 45, and 47 of SEQ ID NO: 25, are as set forth in the following table (Table 15), and the amino acids at positions 16 and 58 are as set forth in Table 14 and described above.

TABLE 15

| | | Preferred amino acid |
|---|---|---|
| Position | Amino acid | in certain embodiments |
| 7 | X can be any amino acid | D, E A, V, or P |
| 8 | X can be any amino acid | A, L, I, V, T or H |
| 9 | X can be any amino acid | T, I |
| 21 | X is A, S, T, V, I, L, Y, or F | F, Y, A, L, or S |
| 22 | X can be any amino acid | H, N, A, K, Q |
| 26 | X is S, T, V, I, L, Y, or F | I, L, Y, V or T |
| 29 | X can be any amino acid | D, E, S, or A |
| 31 | X is L, I, V, Y, F, M, or H | L, Y, I or M |
| 33 | X is S, T or A | S, T or A |
| 41 | X is T or I | T or I |
| 45 | X is F, Y, I, V, L, M or H | Y, F |
| 47 | X is S, T, H or R | S |

In some embodiments, a C3 and/or C3b binding polypeptide comprises an amino acid sequence of SEQ ID NO: 25 or a C3 and/or C3b binding portion thereof (e.g., amino acids 2-59 or 3-59), wherein the amino acid sequence comprises D or A at position 7, A at position 8, T at position 9, S or A at position 21, A or N at position 22, T or V at position 26, D at position 29, L at position 31, S at position 33, I at position 41, Y at position 45, and S at position 47.

In some embodiments, a C3 and/or C3b binding polypeptide is a variant of Sso7d and comprises the amino acid sequence MATVKFXATGEEKEVXISKIXXVYRXGKDILFSYDEGGGKIGWGYVSEKDAPKELLQX EKQKK (SEQ ID NO: 26) or a C3 and/or C3b binding portion thereof (e.g., amino acids 2-59 or 3-59), wherein the amino acids at positions 7, 21, 22, and 26, are as set forth in Table 15 and the amino acids at positions 16 and 58 are as set forth in Table 14.

In some embodiments, a C3 and/or C3b binding polypeptide comprises an amino acid sequence of SEQ ID NO: 26 or a C3 and/or C3b binding portion thereof (e.g., amino acids 2-59 or 3-59), wherein the amino acid sequence comprises D or A at position 7, S or A at position 21, A or N at position 22, T or V at position 26. In some embodiments the polypeptide comprises D at position 7, S at position 21, A at position 22, and T at position 26. In some embodiments the polypeptide comprises A at position 7, A at position 21, N at position 22, and V at position 26.

In some embodiments of SEQ ID NO: 25 or 26 the amino acid at position 16 is D. In some embodiments of SEQ ID NO: 25 or 26 the amino acid at position 16 is E. In some embodiments of SEQ ID NO: 25 or 26 the amino acid at position 58 is M. In some embodiments of SEQ ID NO: 25 or 26 the amino acid at position 58 is L. Amino acids 16 and 58 may thus be DM, DL, EM, or EL in various embodiments.

Where the present disclosure refers to a C3 and/or C3b binding portion of a variant of a Sac7d family member, e.g., a variant represented by SEQ ID NO: 22, SEQ ID NO: 23, SEQ ID NO: 24, SEQ ID NO: 25, SEQ ID NO: 26, SEQ ID NO: 27, SEQ ID NO: 33, or SEQ ID NO: 34, such portion may comprise or consist of amino acids 2-58, 2-59, 2-60, 2-61, 2-62, 2-63, 2-64, 2-65, 1-58, 1-59, 1-60, 1-61, 1-62, 1-63, or 1-64 of the respective sequence in various embodiments. In certain embodiments such portion may comprise or consist of amino acids 3-58, 3-59, 3-60, 3-61, 3-62, 3-63, 3-64, 3-65, or 3-66 of the respective sequence in various embodiments. The disclosure provides embodiments in which such C3 and/or C3b binding portions may be used in fusion proteins, compositions, and/or methods described herein.

C3 and/or C3b binding polypeptides that are variants of any individual member of the Sac7d family may be obtained as described above for Sso7d. By way of further example, variants of Aho7c from Acidianus hospitalis may be generated. Positions 7, 8, 9, 21, 22, 24, 26, 29, 31, 33, 40, 42, 44, and 46 in Sac7d correspond to positions 8, 9, 10, 22, 23, 25, 27, 30, 32, 34, 41, 43, 45, and 47 in Aho7c. Thus, substitutions described above that can be made in the Sac7d sequence at these positions to generate a sequence of a C3 and/or C3b binding polypeptide may be made at positions 8, 9, 10, 22, 23, 25, 27, 30, 32, 34, 41, 43, 45, and 47, respectively, in Aho7c. Thus in some embodiments a C3 and/or C3b binding variant of Aho7c comprises at least 2, 3, 4, 5, 6, 7, 8, 9, 10, 11, 12, 13, or 14 of the following substitutions of SEQ ID NO:14: E, A, or D at position 8; A at position 9; T at position 10; Y, A, or S at position 22; Q, N, or A at position 23; Y at position 25; S, V, or T at position 27; E or D at position 30; A, I, L, V, Y, M, F or H at position 32; A, T or S at position 34; T or I at position 41; W at position 43; A, I, L, V, Y, M, F or H at position 45; and S at position 47. Aho7c has a D at position 17 (corresponding to position 16 in Sac7d) and an N at position 38 (corresponding to position 37 in Sac7d). Thus, any variant of Aho7c may comprise D or E at position 17 (corresponding to position 16 in Sac7d) and/or may comprise N or Q at position 38 (corresponding to position 37 in Sac7d). In some embodiments the amino acids at positions 17 and 38 of a variant of Aho7c are D and N, respectively. In some embodiments the amino acids at positions 17 and 38 of an Aho7c variant are E and N respectively. In some embodiments the amino acids at positions 17 and 38 of an Aho7c variant are D and M, respectively. In some embodiments the amino acids at positions 17 and 38 of an Aho7c variant are D and L, respectively.

In some embodiments, a C3 and/or C3b binding polypeptide comprises an amino acid sequence that is at least 90%, 91%, 92%, 93%, 94%, 95%, 96%, 97%, 98%, 99%, or 100% identical to any one of SEQ ID NOs: 27, 33, 34, 51, or 52 (or a C3 and/or C3b binding portion thereof) listed in Table 16; optionally where the polypeptide does not include the methionine listed at the N-terminal end of each sequence and/or the lysine at the C-terminal end of each sequence. In some embodiments, the C3 and/or C3b binding portion of a C3 and/or C3b binding polypeptide described herein is at least 30, 40, 45, 50, 55, or more amino acid residues in length; optionally, where the polypeptide does not include the methionine listed at the N-terminal end of each sequence and/or the lysine or glutamine at the C-terminal end of each sequence. In some embodiments, any of the C3 and/or C3b binding polypeptides described herein may be modified to not include a methionine at an N-terminal end and/or to not include a lysine or glutamine at a C-terminal end and retain ability to bind to C3 and/or C3b with specificity and/or high affinity, and inhibit complement activation. In some embodiments a C3 and/or C3b binding variant of Sac7d described herein may lack 1, 2, 3, 4, 5, or 6 amino acids corresponding to the 1, 2, 3, 4, 5, or 6 amino acids that are present at the C-terminus of Sac7d and retain ability to bind to C3 and/or C3b with specificity and/or high affinity and inhibit complement activation. In some embodiments any C3 and/or C3b binding variant of Sso7d described herein may lack 1, 2, or 3 amino acids corresponding to the 1, 2, or 3 amino acids that are present at the C-terminus of Sso7d and retain ability to bind to C3 and/or C3b with specificity and/or high affinity and inhibit complement activation.

TABLE 16

| Sequence | |
|---|---|
| Number | Amino Acid Sequences |
| SEQ ID NO: 27 | MVKVKFEATGEEKEVDTSKIYQVYRSGKEVLFTY |
| | DDNGKTGWGYVSEKDAPKELLDMLARAEREK |
| SEQ ID NO: 33 | MVKVKFAATGEEKEVETSKIANVYRVGKDVLFSY |
| | DDQGKIGWGYVSEKDAPKELLDLLARAEREK |
| SEQ ID NO: 34 | MVKVKFDATGEEKEVETSKISAVYRTGKDVLFSY |
| | DDQGKIGWGYVSEKDAPKELLDLLARAEREK |
| SEQ ID NO: 51 | MATVKFDATGEEKEVEISKISAVYRTGKDILFSY |
| | DEGGGKIGWGYVSEKDAPKELLQLLEKQ |
| SEQ ID NO: 52 | MATVKFAATGEEKEVEISKIANVYRVGKDILFSY |
| | DEGGGKIGWGYVSEKDAPKELLQLLEKQ |

Table 17 summarizes amino acids that may be present at positions 8, 9, 10, 22, 23, 25, 27, 30, 32, 34, 42, 44, 46, and/or 48 of a C3 and/or C3b binding Sac7d consensus sequence or corresponding positions in a C3 and/or C3b binding polypeptide that is a variant of Sac7d family member Sac7d, Sso7d, and Aho7c in various embodiments. C3 and/or C3b binding polypeptides that are variants of other Sac7d family members described herein may generated by introducing these amino acids into the sequences of these proteins at corresponding positions. Additionally, any combination of substitutions at positions corresponding to positions 17, 38, and 59 in the Sac7d consensus sequence may be made as described above (e.g., D17E, N38Q, and M59L).

TABLE 17

| Position | | | | | | | | | |
|---|---|---|---|---|---|---|---|---|---|
| in Sac7d | Position | | | | | | | | |
| Consensus | in Sac7d | Position | Position | | | | | | |
| (SEQ ID | (SEQ ID | in Sso7d | in Aho7c | | | | | | |
| NOs 16, | Nos 1, | (SEQ ID | (SEQ ID | | | | | | |
| 76-78) | 65-66) | NO: 2) | NO: 14) | X | X | X | X | X | X |
| 8 | 7 | 7 | 8 | AAA | AAA | D, E A, | AAA | AAA | D, E A, |
| | | | | | | V, or P | | | V, or P |
| 9 | 8 | 8 | 9 | AAA | AAA | A, L, I, | AAA | AAA | A, L, I, |
| | | | | | | V, T or H | | | V, T or H |
| 10 | 9 | 9 | 10 | AAA | AAA | T, I | T | T | T |
| 22 | 21 | 21 | 22 | AAA | A, S, T, | F, Y, A, | AAA | A, S, T, | F, Y, A, |
| | | | | | V, I, L, | L, or S | | V, I, L, | L, or S |
| | | | | | Y, or F | | | Y, or F | |
| 23 | 22 | 22 | 23 | AAA | AAA | H, N, A, | AAA | AAA | H, N, A, |
| | | | | | | K, Q | | | K, Q |
| 25 | 24 | 24 | 25 | Y | Y | Y | Y | Y | Y |
| 27 | 26 | 26 | 27 | AAA | S, T, V, | I, L, Y, | AAA | S, T, V, | I, L, Y, |
| | | | | | I, L, Y, | V or T | | I, L, Y, | V or T |
| | | | | | or F | | | or F | |
| 30 | 29 | 29 | 30 | AAA | AAA | D, E, S, | AAA | AAA | D, E, S, |
| | | | | | | or A | | | or A |
| 32 | 31 | 31 | 32 | A, I, L, | L, I, V, | L, Y, I | L | L | L |
| | | | | V, Y, M, | Y, F, M, | or M | | | |
| | | | | F or H | or H | | | | |
| 34 | 33 | 33 | 34 | S, T | S, T | S, T | S, T, A | S, T, | S, T, |
| | | | | or A | or A | or A | | or A | or A |
| 42 | 40 | 41 | 41 | T or I | T or I | T or I | T or I | T or I | T or I |
| 44 | 42 | 43 | 43 | W | W | W | W | W | W |
| 46 | 44 | 45 | 45 | A, I, L, | F, Y, I, | Y, F | Y | Y | Y |
| | | | | V, Y, M, | V, L, M | | | | |
| | | | | F or H | or H | | | | |
| 48 | 46 | 47 | 47 | AAA | S, T, | S | AAA | S, T, | S |
| | | | | | H or R | | | H or R | |

| Position | | | | | | | | | | | |
|---|---|---|---|---|---|---|---|---|---|---|---|
| in Sac7d | Position | Position | Position | | | | | | | | |
| Consensus | in Sac7d | in Sso7d | in Aho7c | | | | | | | | |
| (SEQ ID 16, | (SEQ ID | (SEQ ID | (SEQ ID | | | | | | | | |
| 76-78, 77) | 1, 65-66) | NO: 2) | NO: 14) | X | X | X | X | X | X | X | X |
| 8 | 7 | 7 | 8 | AAA | AAA | D, E A, | E, A, | A or D | E | A | D |
| | | | | | | V, or P | or D | | | | |
| 9 | 8 | 8 | 9 | A | A | A | A | A | A | A | A |
| 10 | 9 | 9 | 10 | T | T | T | T | T | T | T | T |
| 22 | 21 | 21 | 22 | AAA | A, S, T, | F, Y, A, | Y, A, | A or S | Y | A | S |
| | | | | | V, I, L, | L, or S | or S | | | | |
| | | | | | Y, or F | | | | | | |
| 23 | 22 | 22 | 23 | AAA | AAA | H, N, A, | Q, N, | N or A | Q | N | A |
| | | | | | | K, Q | or A | | | | |
| 25 | 24 | 24 | 25 | Y | Y | Y | Y | Y | Y | Y | Y |
| 27 | 26 | 26 | 27 | AAA | S, T, V, | I, L, Y, | S, V, | V or T | S | V | T |
| | | | | | I, L, Y, | V or T | or T | | | | |
| | | | | | or F | | | | | | |
| 30 | 29 | 29 | 30 | D | D | D | D or E | D | E | D | D |
| 32 | 31 | 31 | 32 | L | L | L | L | L | L | L | L |
| 34 | 33 | 33 | 34 | S | S | S | S or T | S | T | S | S |
| 42 | 40 | 41 | 41 | I | I | I | I | I | T | I | I |
| 44 | 42 | 43 | 43 | W | W | W | W | W | W | W | W |
| 46 | 44 | 45 | 45 | Y | Y | Y | Y | Y | Y | Y | Y |
| 48 | 46 | 47 | 47 | S | S | S | S | S | S | S | S |

Note:
AAA in Table 17 stands for “any amino acid”.

It should be noted that where it is indicated in the present disclosure that a particular amino acid can be any amino acid, in certain embodiments such amino acid is one of the standard 20 amino acids. In certain embodiments, e.g., when a polypeptide is produced by expression in a host cell or in vivo, the amino acids in the polypeptide are standard amino acids in certain embodiments. In certain embodiments, e.g., when a polypeptide is produced using chemical synthesis, one or more non-standard amino acids, which may be analogs of standard amino acids, may be incorporated.

In some embodiments, a C3 and/or C3b binding polypeptide comprises an amino acid sequence that is at least 90%, 91%, 92%, 93%, 94%, 95%, 96%, 97%, 98%, 99%, or 100% identical to SEQ ID NO: 27, or a C3 and/or C3b binding portion thereof (e.g., amino acids 2-58); optionally, where the polypeptide does not include the methionine listed at the N-terminal end of SEQ ID NO: 27 and/or the lysine at the C-terminal end of SEQ ID NO: 27. In some embodiments, a C3 and/or C3b binding polypeptide comprises an amino acid sequence that is at least 90%, 91%, 92%, 93%, 94%, 95%, 96%, 97%, 98%, 99%, or 100% identical to SEQ ID NO: 33, or a C3 and/or C3b binding portion thereof (e.g., amino acids 2-58); optionally, where the polypeptide does not include the methionine listed at the N-terminal end of SEQ ID NO: 33 and/or the lysine at the C-terminal end of SEQ ID NO: 33. In some embodiments, a C3 and/or C3b binding polypeptide comprises an amino acid sequence that is at least 90%, 91%, 92%, 93%, 94%, 95%, 96%, 97%, 98%, 99%, or 100% identical to SEQ ID NO: 34, or a C3 and/or C3b binding portion thereof (e.g., amino acids 2-58); optionally, where the polypeptide does not include the methionine listed at the N-terminal end of each sequence and/or the lysine at the C-terminal end of SEQ ID NO: 34. In some embodiments, a C3 and/or C3b binding polypeptide comprises an amino acid sequence that is at least 90%, 91%, 92%, 93%, 94%, 95%, 96%, 97%, 98%, 99%, or 100% identical to SEQ ID NO: 51, or a C3 and/or C3b binding portion thereof (e.g., amino acids 2-59); optionally, where the polypeptide does not include the methionine listed at the N-terminal end of each sequence. In some embodiments, a C3 and/or C3b binding polypeptide comprises an amino acid sequence that is at least 90%, 91%, 92%, 93%, 94%, 95%, 96%, 97%, 98%, 99%, or 100% identical to SEQ ID NO: 52, or a C3 and/or C3b binding portion thereof (e.g., amino acids 2-59); optionally, where the polypeptide does not include the methionine listed at the N-terminal end of each sequence.

In some embodiments, a C3 and/or C3b binding polypeptide comprises a dimer comprising a first C3 and/or C3b binding polypeptide and a second C3 and/or C3b binding polypeptide, optionally wherein the dimer comprises a linker between the first C3 and/or C3b binding polypeptide and the second C3 and/or C3b binding polypeptide. The first and second C3 and/or C3b binding polypeptides may be the same or different. In embodiments in which they are different, any set of two different C3 and/or C3b binding polypeptides described herein may be used. In some embodiments the C3 and/or C3b polypeptides are covalently joined, e.g., as a fusion protein. In some embodiments the dimer includes a linker (e.g., a flexible linker) between the C3 and/or C3b binding polypeptides. In some embodiments, the first C3 and/or C3b binding polypeptide comprises an amino acid sequence that is at least 90%, 91%, 92%, 93%, 94%, 95%, 96%, 97%, 98%, 99%, or 100% identical to any one of SEQ ID NOs: 27, 33, 34, 51, or 52 (or a portion thereof) listed in Table 16; optionally, where the polypeptide does not include the methionine listed at the N-terminal end of each sequence and/or the lysine or glutamine at the C-terminal end of each sequence. In some embodiments, the C3 and/or C3b binding portion of the first C3 and/or C3b binding polypeptide is at least 10, 20, 30, 40, 45, 50, 55, or more amino acid residues long; optionally, where the polypeptide does not include the methionine listed at the N-terminal end of each sequence and/or the lysine or glutamine at the C-terminal end of each sequence. In some embodiments, the second C3 and/or C3b binding polypeptide comprises an amino acid sequence that is at least 90%, 91%, 92%, 93%, 94%, 95%, 96%, 97%, 98%, 99%, or 100% identical to any one of SEQ ID NOs: 27, 33, 34, 51, or 52 (or a C3 and/or C3b binding portion thereof) listed in Table 16; optionally, where the polypeptide does not include the methionine listed at the N-terminal end of each sequence and/or the lysine or glutamine at the C-terminal end of each sequence. In some embodiments, the C3 and/or C3b binding portion of the first C3 and/or C3b binding polypeptide and the C3 and/or C3b binding portion of the second C3 and/or C3b binding polypeptide is at least 10, 20, 30, 40, 45, 50, 55, or more amino acid residues long; optionally, where the polypeptide does not include the methionine listed at the N-terminal end of each sequence and/or the lysine or glutamine at the C-terminal end of each sequence.

In some embodiments, the first C3 and/or C3b binding polypeptide and/or the second C3 and/or C3b binding polypeptide comprises an amino acid sequence that is at least 90%, 91%, 92%, 93%, 94%, 95%, 96%, 97%, 98%, 99%, or 100% identical to SEQ ID NO: 33, or a C3 and/or C3b binding portion thereof; optionally, where the polypeptide does not include the methionine listed at the N-terminal end of each sequence and/or the lysine at the C-terminal end of SEQ ID NO: 33. In some embodiments, the first C3 and/or C3b binding polypeptide and/or the second C3 and/or C3b binding polypeptide comprises an amino acid sequence that is at least 90%, 91%, 92%, 93%, 94%, 95%, 96%, 97%, 98%, 99%, or 100% identical to SEQ ID NO: 34, or a C3 and/or C3b binding portion thereof; optionally, where the polypeptide does not include the methionine listed at the N-terminal end of each sequence and/or the lysine at the C-terminal end of SEQ ID NO: 34. In some embodiments, the first C3 and/or C3b binding polypeptide and/or the second C3 and/or C3b binding polypeptide comprises an amino acid sequence that is at least 90%, 91%, 92%, 93%, 94%, 95%, 96%, 97%, 98%, 99%, or 100% identical to SEQ ID NO: 51, or a C3 and/or C3b binding portion thereof; optionally, where the polypeptide does not include the methionine listed at the N-terminal end of each sequence. In some embodiments, the first C3 and/or C3b binding polypeptide and/or the second C3 and/or C3b binding polypeptide comprises an amino acid sequence that is at least 90%, 91%, 92%, 93%, 94%, 95%, 96%, 97%, 98%, 99%, or 100% identical to SEQ ID NO: 52, or a C3 and/or C3b binding portion thereof; optionally, where the polypeptide does not include the methionine listed at the N-terminal end of each sequence.

C3 and/or C3b binding polypeptides (or fusion proteins containing one or more C3 and/or C3b binding polypeptides, as described below) can be designed and/or synthesized as mature molecules or precursors (e.g., including a signal peptide). C3 and/or C3b binding polypeptides described herein include both secreted forms and non-secreted forms. In some embodiments, after secretion, a C3 and/or C3b binding polypeptide lacks an N-terminal signal peptide. In some embodiments, prior to secretion, a C3 and/or C3b binding polypeptide includes an N-terminal signal peptide. A signal sequence can be introduced at the N-terminus of a polypeptide comprising a C3 and/or C3b binding polypeptide. In some embodiments a signal sequence found in a mammalian protein, e.g., a human protein, that is secreted may be used. The protein may be, e.g., a cytokine (e.g., an interleukin such as IL-2 or an interferon), a polypeptide hormone (e.g., insulin, growth hormone), an abundant serum protein such as albumin, or a secreted enzyme. One skilled in the art will appreciate that the position of the N-terminal signal peptide can vary and can include, for example, the first 5, 8, 10, 11, 12, 13, 14, 15, 16, 17, 18, 19, 20, 21, 22, 23, 24, 25, 27, 30, or more amino acid residues on the N-terminus of the polypeptide. One of skill in the art can predict the position of a signal sequence cleavage site, e.g., by an appropriate computer algorithm such as that described in Bendtsen et al. (J. Mol. Biol. 340(4):783-795, 2004) and available on the Web at cbs.dtu.dk/services/SignalP/.

### III. FUSION PROTEINS

In some embodiments, the disclosure provides fusion proteins or protein conjugates comprising (i) one or more C3 and/or C3b binding polypeptides described herein; and (ii) a heterologous polypeptide. The one or more C3 and/or C3b binding polypeptide(s) may be any of the C3 and/or C3b binding polypeptides described herein. The one or more C3 and/or C3b binding polypeptides and heterologous polypeptide can be connected by conjugation, e.g., through a covalent bond (see, e.g., Lussow et al., Eur. J. Immun. 21:2297-2302 (1991); Barrios et al., Eur. J. Immun. 22:1365-1372 (1992)), or they can be combined recombinantly to produce a recombinant fusion protein. In some embodiments, the C3 and/or C3b binding polypeptide is fused or conjugated to the N-terminus of the heterologous polypeptide. In some embodiments, the C3 and/or C3b binding polypeptide is fused or conjugated to the C-terminus of the heterologous polypeptide. In some embodiments, a fusion protein or protein conjugate includes a linker (e.g., a flexible linker) between the C3 and/or C3b binding polypeptide and the heterologous polypeptide. In some embodiments, a fusion protein or protein conjugate lacks a linker and a C3 and/or C3b binding polypeptide is fused or conjugated directly to the heterologous polypeptide. The linker may be of any sequence and length that allows the polypeptide to remain biologically active, e.g., not sterically hindered.

Exemplary linker lengths are between 1 and 200 amino acid residues, e.g., 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-110, 111-120, 121-130, 131-140, 141-150, 151-160, 161-170, 171-180, 181-190, or 191-200 amino acid residues. In some embodiments, the linker comprises 1-20 amino acid residues (e.g., 5, 6, 7, 8, 9, 10, 11, 12, 13, 14, 15, 16, 17, 18, 19 or 20 amino acid residues). Linkers are typically between 5 and 30 amino acids in length, e.g., between 10 and 20 amino acids in length, e.g., between 12 and 18 amino acids in length, e.g., 15 amino acids in length. For instance, linkers include or consist of flexible portions, e.g., regions without significant fixed secondary or tertiary structure. Such flexibility is generally increased if the amino acids are small and do not have bulky side chains that impede rotation or bending of the amino acid chain. Thus, preferably a linker of the present disclosure has an increased content of small amino acids, in particular of glycines, alanines, serines, threonines, leucines and isoleucines. For example, the linker may comprise at least 50%, at least 60%, at least 65%, at least 70%, at least 75%, at least 80%, at least 85%, at least 90%, at least 95%, or more glycine, serine, alanine, and/or threonine residues. Linkers may be glycine-rich linkers, e.g., comprising at least 50%, at least 60%, at least 65%, at least 70%, at least 75%, at least 80%, at least 85%, at least 90%, at least 95%, or more glycine residues. Linkers may also be serine-rich linkers, e.g., comprising at least 50%, at least 60%, at least 65%, at least 70%, at least 75%, at least 80%, at least 85%, at least 90%, at least 95%, or more serine residues. In certain embodiments, the linker comprises at least 80%, at least 85%, at least 90%, at least 95%, or more glycine, serine, alanine, and/or threonine residues, and the remaining residues, if any, are glutamine, phenylalanine, and/lysine. In some embodiments, the linker comprises (GGGGS)n where n=1, 2, 3, 4, or 5 (SEQ ID NO: 87), or permuted versions thereof, e.g., (SGGGG)n where n=1, 2, 3, 4, or 5 (SEQ ID NO: 88). In some embodiments, the linker comprises (G)n where n is between 5 and 25 (SEQ ID NO: 89), e.g., between 5 and 15. Exemplary flexible linkers comprise the amino acid sequence of GSGGSGSAGSGSGGS (SEQ ID NO: 41), GSAGSAAGSGEF (SEQ ID NO: 42), or EGKSSGSGSESKST (SEQ ID NO: 43). In certain embodiments, the linker comprises the amino acid sequence of GSGGSGSAGSGSGGS (SEQ ID NO: 41).

In certain embodiments a heterologous polypeptide may be up to about 1,000 amino acids long. In certain embodiments a heterologous polypeptide is at least 10 amino acids long, e.g., between 10 and 100 amino acids long, between 100 and 250 amino acids long, between 250 and 500 amino acids long, between 500 and 1000 amino acids long.

In some embodiments, a heterologous polypeptide binds to a one or more target polypeptides of interest. In some embodiments, a heterologous polypeptide reduces expression and/or activity of one or more target polypeptides of interest. In some embodiments, a heterologous polypeptide is a Nanofitin® that is not a C3 and/or C3b binding polypeptide described herein. In certain embodiments a target polypeptide of interest is a protein that promotes angiogenesis, e.g., a component of the vascular endothelial growth factor (VEGF) pathway, e.g., VEGF-A or a VEGF-A receptor.

In some embodiments, a heterologous polypeptide described herein is an antibody chain polypeptide (or fragment thereof). In some embodiments, the disclosure provides antibody fusion proteins or antibody fragment fusion proteins (e.g., Fab fusion proteins), which include one or more C3 and/or C3b binding polypeptides fused to one or more antibody chain polypeptides (or fragments thereof). Antibodies or antibody fragments include, e.g., an antigen-binding region of an immunoglobulin, a heavy chain of an antibody, light chain of an antibody, LRR-based antibody, or other protein scaffold with antibody-like properties, as well as other antigen binding moieties known in the art, including, e.g., a Fab, Fab′, Fab′2, Fab₂, Fab₃, F(ab′)₂, Fd, Fv, Feb, scFv, SMIP, antibody, diabody, triabody, tetrabody, minibody, maxibody, tandab, DVD, BiTe, TandAb, or the like) or one or more variable domains described herein, or a portion thereof (e.g., one or more CDRs described herein)). In some embodiments a C3 and/or C3b binding polypeptide may be fused to the N-terminus of a heavy chain of an antibody or antibody fragment (e.g., a Fab). In some embodiments a C3 and/or C3b binding polypeptide may be fused to the N-terminus of light chain of an antibody or antibody fragment (e.g., a Fab). In some embodiments a C3 and/or C3b binding polypeptide may be fused to the C-terminus of a heavy chain of an antibody or antibody fragment (e.g., a Fab). In some embodiments a C3 and/or C3b binding polypeptide may be fused to the C-terminus of a light chain of an antibody or antibody fragment (e.g., a Fab).

In some embodiments, a heterologous polypeptide comprises an angiogenesis inhibitor, i.e., an agent that inhibits the growth of new blood vessels. Angiogenesis inhibitors are capable of inhibiting or reducing one or more processes associated with angiogenesis including, but not limited to, endothelial cell proliferation, endothelial cell migration, and/or capillary tube formation. In some embodiments, such agents may reduce vascular permeability and thereby inhibit fluid exudation from blood vessels. The vascular endothelial growth factor (VEGF) pathway plays a major role in angiogenesis. Suitable angiogenesis inhibitors include, but are not limited to, inhibitors of the VEGF pathway, e.g., inhibitors of VEGFR1 tyrosine kinase activity; inhibitors of VEGFR2 tyrosine kinase activity; antibodies that bind to VEGF; antibodies that bind to VEGFR1; antibodies that bind to VEGFR2; a soluble VEGFR; and the like (see, e.g., Takayama et al. (2000) Cancer Res. 60:2169-2177; Mori et al. (2000) Gene Ther. 7:1027-1033; and Mahasreshti et al. (2001) Clin. Cancer Res. 7:2057-2066; and U.S. Patent Publication No. 20030181377). In certain embodiments, the angiogenesis inhibitor inhibits one or more VEGF family members (e.g., VEGF-A, PGF, VEGF-B, VEGF-C, and/or VEGF-D). In certain embodiments, the angiogenesis inhibitor is a VEGF inhibitor. As used herein, the term “VEGF inhibitor” refers to an agent (e.g., a protein) that binds to one or more VEGF family members (e.g., VEGF-A) and inhibits one or more biological activities of such VEGF family member, e.g., promotion of vascular endothelial cell proliferation, migration, and/or capillary tube formation. In some embodiments the VEGF inhibitor binds to the receptor binding site of active forms of VEGF-A and prevents interaction of VEGF-A with its receptors (VEGFR1 and VEGFR2) on the surface of endothelial cells. In some embodiments the VEGF inhibitor comprises an antibody or antibody fragment (e.g., a Fab or an scFv) that binds to VEGF-A.

In some embodiments, a heterologous polypeptide described herein is or comprises a polypeptide chain that is included in an antibody or antibody fragment. In certain embodiments, the antibody (or fragment thereof) comprises a heavy chain fragment having the amino acid sequence of SEQ ID NO: 36 (or a VEGF-binding fragment thereof) and/or a light chain having the amino acid sequence of SEQ ID NO: 35 (or a VEGF-binding fragment thereof) (Table 18). In some embodiments, the antibody (or VEGF-binding fragment thereof) comprises a heavy chain fragment having an amino acid sequence at least 90%, 91%, 92%, 93%, 94%, 95%, 96%, 97%, 98%, or 99% identical to SEQ ID NO: 36 (or a VEGF-binding fragment thereof); and/or a light chain having an amino acid sequence at least 90%, 91%, 92%, 93%, 94%, 95%, 96%, 97%, 98%, or 99% identical to SEQ ID NO: 35 (or VEGF-binding fragment thereof). In certain embodiments, the antibody is ranibizumab (also known as rhuFAb V2 or Lucentis® (Genentech)) or a VEGF-binding fragment thereof. Ranibizumab is known in the art and is a monoclonal antibody fragment (Fab) created from the same parent mouse antibody as bevacizumab. Ranibizumab is approved for treatment of wet AMD. Ranibizumab is described in Papadopoulos et al. (2012). Binding and neutralization of vascular endothelial growth factor (VEGF) and related ligands by VEGF Trap, ranibizumab and bevacizumab. Angiogenesis. 15. 171-85, which is hereby incorporated by reference in its entirety. In some embodiments the antibody competes with ranibizumab for binding to VEGF-A.

In some embodiments, a Fab fusion protein comprises a C3 and/or C3b binding polypeptide fused to the N-terminus of the heavy chain of ranibizumab. In some embodiments, a Fab fusion protein comprises a C3 and/or C3b binding polypeptide fused to the N-terminus of the light chain of ranibizumab. In some embodiments, a Fab fusion protein comprises a C3 and/or C3b binding polypeptide fused to the C-terminus of the heavy chain of ranibizumab. In some embodiments, a Fab fusion protein comprises a C3 and/or C3b binding polypeptide fused to the C-terminus of the light chain of ranibizumab. In some embodiments, a Fab fusion protein comprises a peptide linker between the heavy chain or the light chain of ranibizumab and the C3 and/or C3b binding polypeptide.

In some embodiments, a Fab fusion protein comprises or consists of the amino acid sequence of any one of SEQ ID NOs: 37-40 or 55-63. In some embodiments, a Fab fusion protein comprises or consists of an amino acid sequence at least 90%, 91%, 92%, 93%, 94%, 95%, 96%, 97%, 98%, or 99% identical to one of SEQ ID NOs: 37-40 or 55-63. In certain embodiments, a Fab fusion protein comprises or consists of the amino acid sequence of SEQ ID NO: 37. In certain embodiments, a Fab fusion protein comprises or consists of the amino acid sequence of SEQ ID NO: 38. In certain embodiments, a Fab fusion protein comprises or consists of the amino acid sequence of SEQ ID NO: 39. In certain embodiments, a Fab fusion protein comprises or consists of the amino acid sequence of SEQ ID NO: 40. In certain embodiments, a Fab fusion protein comprises or consists of the amino acid sequence of SEQ ID NO: 55. In certain embodiments, a Fab fusion protein comprises or consists of the amino acid sequence of SEQ ID NO: 56. In certain embodiments, a Fab fusion protein comprises or consists of the amino acid sequence of SEQ ID NO: 57. In certain embodiments, a Fab fusion protein comprises or consists of the amino acid sequence of SEQ ID NO: 58. In certain embodiments, a Fab fusion protein comprises or consists of the amino acid sequence of SEQ ID NO: 59. In certain embodiments, a Fab fusion protein comprises or consists of the amino acid sequence of SEQ ID NO: 60. In certain embodiments, a Fab fusion protein comprises or consists of the amino acid sequence of SEQ ID NO: 61. In certain embodiments, a Fab fusion protein comprises or consists of the amino acid sequence of SEQ ID NO: 62. In certain embodiments, a Fab fusion protein comprises or consists of the amino acid sequence of SEQ ID NO: 63.

TABLE 18

| Sequence | | |
|---|---|---|
| Number | Description | Amino Acid Sequence |
| SEQ ID NO: 35 | Ranibizumab Light | DIQLTQSPSSLSASVGDRVTITCSASQDISNYLNWYQQK |
| | Chain | PGKAPKVLIYFTSSLHSGVPSRESGSGSGTDFTLTISSL |
| | | QPEDFATYYCQQYSTVPWTFGQGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 36 | Ranibizumab | EVQLVESGGGLVQPGGSLRLSCAASGYDFTHYGMNWVRQ |
| | Heavy Chain | APGKGLEWVGWINTYTGEPTYAADFKRRFTFSLDTSKST |
| | | AYLQMNSLRAEDTAVYYCAKYPYYYGTSHWYFDVWGQGT |
| | | LVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKKVEPKSCDKTHL |
| SEQ ID NO: 37 | Ranibizumab Light | DIQLTQSPSSLSASVGDRVTITCSASQDISNYLNWYQQK |
| | Chain fused to | PGKAPKVLIYFTSSLHSGVPSRFSGSGSGTDFTLTISSL |
| | SEQ ID NO: 34 | QPEDFATYYCQQYSTVPWTFGQGTKVEIKRTVAAPSVFI |
| | lacking M at the | FPPSDEQLKSGTASVVCLLNNFYPREAKVQWKVDNALQS |
| | N-terminus of SEQ | GNSQESVTEQDSKDSTYSLSSTLTLSKADYEKHKVYACE |
| | ID NO: 34 | VTHQGLSSPVTKSFNRGECGSGGSGSAGSGSGGSVKVKF |
| | (linker region | DATGEEKEVETSKISAVYRTGKDVLFSYDDQGKIGWGYV |
| | underlined and | SEKDAPKELLDLLARAEREK |
| | bolded) | |
| SEQ ID NO: 38 | Ranibizumab | EVQLVESGGGLVQPGGSLRLSCAASGYDFTHYGMWVRQA |
| | Heavy Chain fused | PGKGLEWVGWINTYTGEPTYAADFKRRFTFSLDTSKSTA |
| | to SEQ ID NO: 34 | YLQMNSLRAEDTAVYYCAKYPYYYGTSHWYFDVWGQGTL |
| | lacking M at the | VTVSSASTKGPSVFPLAPSSKSTSGGTAALGCLVKDYFP |
| | N-terminus of SEQ | EPVTVSWNSGALTSGVHTFPAVLQSSGLYSLSSVVTVPS |
| | ID NO: 34 | SSLGTQTYICNVNHKPSNTKVDKKVEPKSCDKTHLGSGG |
| | (linker region | SGSAGSGSGGSVKVKFDATGEEKEVETSKISAVYRTGKD |
| | underlined and | VLFSYDDQGKIGWGYVSEKDAPKELLDLLARAEREK |
| | bolded) | |
| SEQ ID NO: 39 | Ranibizumab Light | DIQLTQSPSSLSASVGDRVTITCSASQDISNYLNWYQQK |
| | Chain fused to | PGKAPKVLIYFTSSLHSGVPSRFSGSGSGTDFTLTISSL |
| | SEQ ID NO: 27 | QPEDFATYYCQQYSTVPWTFGQGTKVEIKRTVAAPSVFI |
| | lacking M at the | FPPSDEQLKSGTASVVCLLNNFYPREAKVQWKVDNALQS |
| | N-terminus of SEQ | GNSQESVTEQDSKDSTYSLSSTLTLSKADYEKHKVYACE |
| | ID NO: 27 | VTHQGLSSPVTKSFNRGECGSGGSGSAGSGSGGSVKVKF |
| | (linker region | EATGEEKEVDTSKIYQVYRSGKEVLFTYDDNGKTGWGYV |
| | underlined and | SEKDAPKELLDMLARAEREK |
| | bolded) | |
| SEQ ID NO: 40 | Ranibizumab | EVQLVESGGGLVQPGGSLRLSCAASGYDFTHYGMNWVRQ |
| | Heavy Chain fused | APGKGLEWVGWINTYTGEPTYAADFKRRFTFSLDTSKST |
| | to SEQ ID NO: 27 | AYLQMNSLRAEDTAVYYCAKYPYYYGTSHWYFDVWGQGT |
| | lacking M at the | LVTVSSASTKGPSVFPLAPSSKSTSGGTAALGCLVKDYF |
| | N-terminus of SEQ | PEPVTVSWNSGALTSGVHTFPAVLQSSGLYSLSSVVTVP |
| | ID NO: 27 | SSSLGTQTYICNVNHKPSNTKVDKKVEPKSCDKTHLGSG |
| | (linker region | GSGSAGSGSGGSVKVKFEATGEEKEVDTSKIYQVYRSGK |
| | underlined and | EVLFTYDDNGKTGWGYVSEKDAPKELLDMLARAEREK |
| | bolded) | |
| SEQ ID NO: 55 | Ranibizumab Light | DIQLTQSPSSLSASVGDRVTITCSASQDISNYLNWYQQK |
| | Chain fused to | PGKAPKVLIYFTSSLHSGVPSRFSGSGSGTDFTLTISSL |
| | SEQ ID NO: 33 | QPEDFATYYCQQYSTVPWTFGQGTKVEIKRTVAAPSVFI |
| | lacking M at the | FPPSDEQLKSGTASVVCLLNNFYPREAKVQWKVDNALQS |
| | N-terminus of SEQ | GNSQESVTEQDSKDSTYSLSSTLTLSKADYEKHKVYACE |
| | ID NO: 33 | VTHQGLSSPVTKSFNRGECGSGGSGSAGSGSGGSVKVKF |
| | (linker region | AATGEEKEVETSKIANVYRVGKDVLFSYDDQGKIGWGYV |
| | underlined and | SEKDAPKELLDLLARAEREK |
| | bolded) | |
| SEQ ID NO: 56 | Ranibizumab | EVQLVESGGGLVQPGGSLRLSCAASGYDFTHYGMNWVRQ |
| | Heavy Chain fused | APGKGLEWVGWINTYTGEPTYAADFKRRFTFSLDTSKST |
| | to SEQ ID NO: 33 | AYLQMNSLRAEDTAVYYCAKYPYYYGTSHWYFDVWGQGT |
| | lacking M at the | LVTVSSASTKGPSVFPLAPSSKSTSGGTAALGCLVKDYF |
| | N-terminus of SEQ | PEPVTVSWNSGALTSGVHTFPAVLQSSGLYSLSSVVTVP |
| | ID NO: 33 | SSSLGTQTYICNVNHKPSNTKVDKKVEPKSCDKTHLGSG |
| | (linker region | GSGSAGSGSGGSVKVKFAATGEEKEVETSKIANVYRVGK |
| | underlined and | DVLFSYDDQGKIGWGYVSEKDAPKELLDLLARAEREK |
| | bolded) | |
| SEQ ID NO: 57 | Ranibizumab Light | DIQLTQSPSSLSASVGDRVTITCSASQDISNYLNWYQQK |
| | Chain fused to | PGKAPKVLIYFTSSLHSGVPSRFSGSGSGTDFTLTISSL |
| | SEQ ID NO: 34 | QPEDFATYYCQQYSTVPWTFGQGTKVEIKRTVAAPSVFI |
| | (lacking M at the | FPPSDEQLKSGTASVVCLLNNFYPREAKVQWKVDNALQS |
| | N terminus and K | GNSQESVTEQDSKDSTYSLSSTLTLSKADYEKHKVYACE |
| | at the C terminus | VTHQGLSSPVTKSFNRGECGSGGSGSAGSGSGGSVKVKF |
| | of SEQ ID NO: | DATGEEKEVETSKISAVYRTGKDVLFSYDDQGKIGWGYV |
| | 34) (linker region | SEKDAPKELLDLLARAERE |
| | underlined and | |
| | bolded) | |
| SEQ ID NO: 58 | Ranibizumab | EVQLVESGGGLVQPGGSLRLSCAASGYDFTHYGMNWVRQ |
| | Heavy Chain fused | APGKGLEWVGWINTYTGEPTYAADFKRRFTFSLDTSKST |
| | to SEQ ID NO: 34 | AYLQMNSLRAEDTAVYYCAKYPYYYGTSHWYFDVWGQGT |
| | (lacking M at the | LVTVSSASTKGPSVFPLAPSSKSTSGGTAALGCLVKDYF |
| | N terminus and K | PEPVTVSWNSGALTSGVHTFPAVLQSSGLYSLSSVVTVP |
| | at the C terminus | SSSLGTQTYICNVNHKPSNTKVDKKVEPKSCDKTHLGSG |
| | of SEQ ID NO: | GSGSAGSGSGGSVKVKFDATGEEKEVETSKISAVYRTGK |
| | 34) (linker region | DVLFSYDDQGKIGWGYVSEKDAPKELLDLLARAERE |
| | underlined and | |
| | bolded) | |
| SEQ ID NO: 59 | Ranibizumab | EVQLVESGGGLVQPGGSLRLSCAASGYDFTHYGMNWVRQ |
| | Heavy Chain fused | APGKGLEWVGWINTYTGEPTYAADFKRRFTFSLDTSKST |
| | to SEQ ID NO: 73 | AYLQMNSLRAEDTAVYYCAKYPYYYGTSHWYFDVWGQGT |
| | (lacking K at the | LVTVSSASTKGPSVFPLAPSSKSTSGGTAALGCLVKDYF |
| | C terminus of SEQ | PEPVTVSWNSGALTSGVHTFPAVLQSSGLYSLSSVVTVP |
| | ID NO: 73) (linker | SSSLGTQTYICNVNHKPSNTKVDKKVEPKSCDKTHLGSG |
| | region underlined | GSGSAGSGSGGSVKVKFVFSGEEKEVETSKIKWVLRWGK |
| | and bolded) | AVGFKYDDQGKIGYGFVAEKDAPKELLDLLARAERE |
| SEQ ID NO: 60 | Ranibizumab Light | DIQLTQSPSSLSASVGDRVTITCSASQDISNYLNWYQQK |
| | Chain fused to | PGKAPKVLIYFTSSLHSGVPSRESGSGSGTDFTLTISSL |
| | SEQ ID NO: 34 | QPEDFATYYCQQYSTVPWTFGQGTKVEIKRTVAAPSVFI |
| | (lacking M at the | FPPSDEQLKSGTASVVCLLNNFYPREAKVQWKVDNALQS |
| | N terminus of SEQ | GNSQESVTEQDSKDSTYSLSSTLTLSKADYEKHKVYACE |
| | ID NO: 34) fused | VTHQGLSSPVTKSFNRGECGSGGSGSAGSGSGGSVKVKF |
| | to SEQ ID NO: 73 | DATGEEKEVETSKISAVYRTGKDVLFSYDDQGKIGWGYV |
| | (lacking K at the | SEKDAPKELLDLLARAEREKGSGGSGSAGSGSGGSVKVK |
| | C terminus of SEQ | FVFSGEEKEVETSKIKWVLRWGKAVGFKYDDQGKIGYGF |
| | ID NO: 73) (linker | VAEKDAPKELLDLLARAERE |
| | regions underlined | |
| | and bolded) | |
| SEQ ID NO: 61 | Ranibizumab | EVQLVESGGGLVQPGGSLRLSCAASGYDFTHYGMNWVRQ |
| | Heavy Chain fused | APGKGLEWVGWINTYTGEPTYAADFKRRFTFSLDTSKST |
| | to SEQ ID NO: 34 | AYLQMNSLRAEDTAVYYCAKYPYYYGTSHWYFDVWGQGT |
| | (lacking M at the | LVTVSSASTKGPSVFPLAPSSKSTSGGTAALGCLVKDYF |
| | N terminus of SEQ | PEPVTVSWNSGALTSGVHTFPAVLQSSGLYSLSSVVTVP |
| | ID NO: 34) fused | SSSLGTQTYICNVNHKPSNTKVDKKVEPKSCDKTHLGSG |
| | to SEQ ID NO: 73 | GSGSAGSGSGGSVKVKFDATGEEKEVETSKISAVYRTGK |
| | (lacking K at the | DVLFSYDDQGKIGWGYVSEKDAPKELLDLLARAEREKGS |
| | C terminus of SEQ | GGSGSAGSGSGGSVKVKFVFSGEEKEVETSKIKWVLRWG |
| | ID NO: 73) (linker | KAVGFKYDDQGKIGYGFVAEKDAPKELLDLLARAERE |
| | regions underlined | |
| | and bolded) | |
| SEQ ID NO: 62 | Ranibizumab Light | DIQLTQSPSSLSASVGDRVTITCSASQDISNYLNWYQQK |
| | Chain fused to | PGKAPKVLIYFTSSLHSGVPSRFSGSGSGTDFTLTISSL |
| | SEQ ID NO: 73 | QPEDFATYYCQQYSTVPWTFGQGTKVEIKRTVAAPSVFI |
| | fused to SEQ ID | FPPSDEQLKSGTASVVCLLNNFYPREAKVQWKVDNALQS |
| | NO: 34 (lacking M | GNSQESVTEQDSKDSTYSLSSTLTLSKADYEKHKVYACE |
| | at the N terminus | VTHQGLSSPVTKSFNRGECGSGGSGSAGSGSGGSVKVKF |
| | and K at the C | VFSGEEKEVETSKIKWVLRWGKAVGFKYDDQGKIGYGFV |
| | terminus of SEQ | AEKDAPKELLDLLARAEREKGSGGSGSAGSGSGGSVKVK |
| | ID NO: 34) (linker | FDATGEEKEVETSKISAVYRTGKDVLFSYDDQGKIGWGY |
| | regions underlined | VSEKDAPKELLDLLARAERE |
| | and bolded) | |
| SEQ ID NO: 63 | Ranibizumab | EVQLVESGGGLVQPGGSLRLSCAASGYDFTHYGMNWVRQ |
| | Heavy Chain fused | APGKGLEWVGWINTYTGEPTYAADFKRRFTFSLDTSKST |
| | to SEQ ID NO: 73 | AYLQMNSLRAEDTAVYYCAKYPYYYGTSHWYFDVWGQGT |
| | fused to SEQ ID | LVTVSSASTKGPSVFPLAPSSKSTSGGTAALGCLVKDYF |
| | NO: 34 (lacking M | PEPVTVSWNSGALTSGVHTFPAVLQSSGLYSLSSVVTVP |
| | at the N terminus | SSSLGTQTYICNVNHKPSNTKVDKKVEPKSCDKTHLGSG |
| | and K at the C | GSGSAGSGSGGSVKVKFVFSGEEKEVETSKIKWVLRWGK |
| | terminus of SEQ | AVGFKYDDQGKIGYGFVAEKDAPKELLDLLARAEREKGS |
| | ID NO: 34) (linker | GGSGSAGSGSGGSVKVKFDATGEEKEVETSKISAVYRTG |
| | regions underlined | KDVLFSYDDQGKIGWGYVSEKDAPKELLDLLARAERE |
| | and bolded) | |

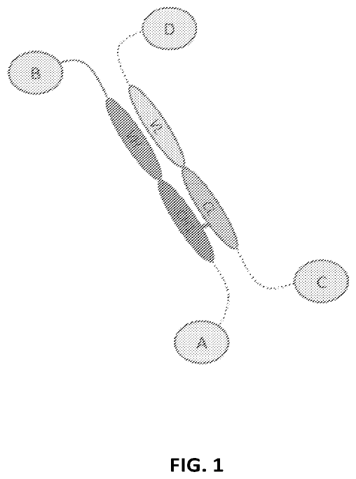

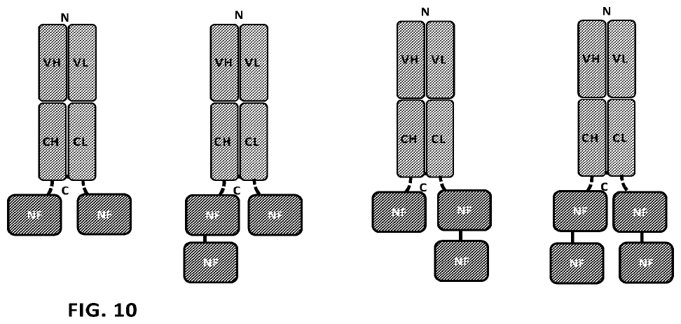

In some aspects, described herein is a protein comprising a modified Fab (e.g., ranibizumab) that binds to VEGF-A, wherein a C3 and/or C3b binding polypeptide is fused to the heavy chain or the light chain of the Fab. In some embodiments the C3 and/or C3b binding polypeptide is fused to the heavy or light chain of the Fab at position A, B, C, or D (where positions are as shown in FIG. 1). In some embodiments the heavy chain or the light chain of the Fab and the C3 and/or C3b binding polypeptide are joined by a peptide linker to form a linear polypeptide. In some embodiments the modified Fab comprises SEQ ID NO: 35 and SEQ ID NO: 38. In some embodiments the modified Fab comprises SEQ ID NO: 35 and SEQ ID NO: 40. In some embodiments the modified Fab comprises SEQ ID NO: 35 and SEQ ID NO: 56. In some embodiments the modified Fab comprises SEQ ID NO: 36 and SEQ ID NO: 37. In some embodiments the modified Fab comprises SEQ ID NO: 36 and SEQ ID NO: 39. In some embodiments the modified Fab comprises SEQ ID NO: 36 and SEQ ID NO: 55. In some embodiments the modified Fab may have two, three, or four C3 and/or C3b binding polypeptides fused thereto. The C3 and/or C3b binding polypeptides may be attached at any one or more of positions A, B, C, and D shown in FIG. 1. In some embodiments two or more C3 and/or C3b binding polypeptides may be joined as a linear polypeptide, which is attached at position A, B, C, or D shown in FIG. 1. FIG. 10 shows schematic diagrams of certain proteins comprising a Fab and two, three, or four C3 and/or C3b binding polypeptides. In the proteins depicted in FIG. 10, the C3 and/or C3b binding polypeptides are attached at the C-terminal end of the heavy or light chain of the Fab (positions A and C in FIG. 1). In some embodiments, one or more C3 and/or C3b binding polypeptide(s) may additionally or alternately be attached at the N-terminal end of the heavy and/or light chain of the Fab (positions B and D in FIG. 1). The C3 and/or C3b binding polypeptides in a protein comprising multiple C3 and/or C3b binding polypeptides may be the same or different. In certain embodiments a modified Fab comprises two or more instances of the C3 and/or C3b binding polypeptide of SEQ ID NO: 33 or a C3 and/or C3b binding fragment thereof. In certain embodiments a modified Fab comprises two or more instances of the C3 and/or C3b binding polypeptide of SEQ ID NO: 33 or SEQ ID NO: 34 or a C3 and/or C3b binding portion thereof. Peptide linkers may be located between any two polypeptides that are part of a longer polypeptide. In some embodiments the N-terminal methionine and/or one or more amino acids at the C-terminal end of SEQ ID NO: 33 or SEQ ID NO: 34 may be omitted, as described above. While proteins comprising a Fab and a C3 and/or C3b binding polypeptide are of particular interest herein, the disclosure provides analogous embodiments in which a different antibody fragment containing an antigen-binding domain, or a full size antibody, is used instead of a Fab.

In certain embodiments, the heterologous polypeptide is or comprises a bevacizumab (Avastin®; Roche) polypeptide chain, or a VEGF-binding fragment thereof. Bevacizumab is a humanized monoclonal IgG1 antibody that binds to and inhibits the activity of VEGF. Bevacizumab contains human framework regions and the complementarity-determining regions of a murine antibody that binds to VEGF. Bevacizumab is approved for treatment of various cancers and has been widely used for treatment of neovascular (wet) AMD. Bevacizumab is described in Papadopoulos et al. (2012). Binding and neutralization of vascular endothelial growth fact (VEGF) and related ligands by VEGF Trap, ranibizumab and bevacizumab. Angiogenesis. 15: 171-85, which is hereby incorporated by reference in its entirety. In some embodiments, the disclosure provides a modified bevacizumab, or fragment thereof, comprising a C3 and/or C3b binding polypeptide described herein.

In certain embodiments, the heterologous polypeptide is or comprises an aflibercept (Eylea®; Regeneron) polypeptide chain, or a VEGF-binding portion thereof. Aflibercept is also known as VEGF-Trap or VEGF-Trap Eye. Aflibercept is a fusion protein containing VEGF-binding portions from the extracellular domains of human VEGF receptors 1 and 2 (the second extracellular domain of human VEGFR-1 and the third extracellular domain of human VEGFR-2) fused to the Fc portion of human immunoglobulin IgG1. Aflibercept is approved for the treatment of wet AMD. Aflibercept is a decoy receptor that binds to VEGF, thereby preventing VEGF from binding to its receptors. Aflibercept (VEGF-Trap) is also described in Papadopoulos et al. (2012). Binding and neutralization of vascular endothelial growth fact (VEGF) and related ligands by VEGF Trap, ranibizumab and bevacizumab. Angiogenesis. 15: 171-85, which is hereby incorporated by reference in its entirety. The amino acid sequence of Aflibercept (VEGF-Trap) is described in Holash et al. (2002) VEGF-Trap: a VEGF blocker with potent antitumor effects. Proc Natl Acad Sci, 99: 11393-11398, which is hereby incorporated by reference in its entirety. In some embodiments, the disclosure provides a modified aflibercept, or fragment thereof, comprising a C3 and/or C3b binding polypeptide described herein.

In certain embodiments, the heterologous polypeptide is or comprises a brolucizumab (Beovu®; Novartis) polypeptide chain, or a VEGF-binding portion thereof. Brolucizumab is a humanized scFv that binds to and inhibits VEGF-A. Brolucizumab is approved for the treatment of wet AMD. Brolucizumab is described in Yannuzzi & Freund (2019). Brolucizumab: evidence to date in the treatment of neovascular age-related macular degeneration. Clinical Ophthalmology. 13: 1323-1329, which is hereby incorporated by reference in its entirety. In some embodiments, the disclosure provides a modified brolucizumab, or fragment thereof, comprising a C3 and/or C3b binding polypeptide described herein.

In certain embodiments, the heterologous polypeptide is or comprises abicipar (Allergan plc/Molecular Partners; CAS: 1327278-94-3), or a VEGF-binding portion thereof. Abicipar is also known as MP0112. Abicipar is a designed ankyrin repeat protein (DARPin) that specifically binds with high affinity to all soluble isoforms of VEGF-A. Abicipar is described in Kunimoto et al. (2019). Evaluation of Abicipar Pegol (an Anti-VEGF DARPin Therapeutic) in Patients With Neovascular Age-Related Macular Degeneration: Studies in Japan and the United States. Ophthalmic Surgery, Lasers and Imaging Retina. 50: e10-e22, and in Souied et al. (2014), Treatment of Exudative Age-Related Macular Degeneration with a Designed Ankyrin Repeat Protein that Binds Vascular Endothelial Growth Factor: A Phase I/II Study, American Journal of Ophthalmology. 158: 724-732; which are hereby incorporated by reference in their entirety.

In certain embodiments, the heterologous polypeptide is or comprises a Conbercept (Lumitin®; Chengdu Kanghong Biotechnologies) polypeptide chain, or a VEGF-binding portion thereof, and is a decoy receptor. Conbercept is also known as KH902. Conbercept is a fusion protein containing VEGF-binding portions from the extracellular domains of human VEGF receptors 1 and 2 (second extracellular domain of human VEGFR-1 and the third and fourth extracellular domains of human VEGFR-2) fused to the Fc portion of human immunoglobulin IgG1. Conbercept is approved in China for treatment of wet AMD and is in clinical trials aiming towards US approval. Conbercept is described in Wang et al. (2013). Novel VEGF Decoy Receptor Fusion Protein Conbercept Targeting Multiple VEGF Isoforms Provide Remarkable Anti-Angiogenesis Effect In Vivo. PloS one. 8: e70544, which is hereby incorporated by reference in its entirety. Conbercept is also described in Wu et al. (2019). Conbercept is also described in Wu et al. Intravitreal conbercept injection for neovascular age-related macular degeneration. International Journal of Ophthalmology. 12: 252-257, which is hereby incorporated by reference in its entirety. In some embodiments, the disclosure provides a modified Conbercept, or fragment thereof, comprising a C3 and/or C3b binding polypeptide described herein.

In some embodiments a protein comprising (i) a C3 and/or C3b binding polypeptide and (ii) an angiogenesis inhibitor (e.g., a VEGF inhibitor) inhibits angiogenesis with an activity at least 50%, 60%, 70%, 80%, 90%, or more of that of the angiogenesis inhibitor alone on a molar basis, e.g., the activity of the protein is between 50% and about 100% of the activity of the angiogenesis inhibitor alone on a molar basis. For example, in some embodiments a protein comprising a modified Fab described herein inhibits angiogenesis with an activity at least 50%, 60%, 70%, 80%, 90%, or more of that of the Fab alone on a molar basis, e.g., the activity of the modified Fab is between 50% and about 100% of that of the VEGF inhibitor alone on a molar basis. In some embodiments a protein comprising a modified Fab described herein inhibits angiogenesis with an activity at least 50%, 60%, 70%, 80%, 90%, or more of that of ranibizumab on a molar basis, e.g., the activity of the modified Fab is between 50% and about 100% of that of ranibizumab on a molar basis. In some embodiments the angiogenesis-inhibiting activity of an agent (e.g., a modified Fab or modified scFv) may be measured using an assay that measures the ability of an agent to inhibit VEGF-induced (e.g., VEGF-A induced) proliferation of endothelial cells (e.g., human umbilical vein endothelial cells). An exemplary assay is described in the Examples.

In some embodiments, the heterologous polypeptide comprises a polypeptide that extends half-life of a C3 and/or C3b binding polypeptide, e.g., relative to the C3 and/or C3b binding polypeptide without the heterologous polypeptide. Such a polypeptide may be referred to herein as a “half-life extending polypeptide” (HEP), and any of the following heterologous polypeptides can be a HEP. In some embodiments, the heterologous polypeptide is a transferrin polypeptide or a portion thereof. Transferrin is recycled by binding to a transferrin receptor (see, e.g., Widera et al., Adv. Drug Deliv. Rev. 55:1439-66 (2003)). In some embodiments, the heterologous polypeptide comprises an Fc domain of an immunoglobulin or a portion thereof. In some embodiments the Fc domain contains one or more mutations that substantially reduces or eliminates one or more effector functions of a wild type Fc domain. In certain embodiments, the Fc domain or a portion thereof comprises one or more amino acid substitutions (e.g., one, two, three, four, five, six, or seven of L234A, L235A, G237A, N297A, E318A, K320A, or K322A). In some embodiments, the heterologous polypeptide is albumin (e.g., bovine serum albumin (BSA), human serum albumin (HSA), or mouse serum albumin (MSA)) or a fragment thereof. In some embodiments, the heterologous polypeptide is a polypeptide that binds a serum protein. In some embodiments, the heterologous polypeptide is a serum albumin binder (e.g., a BSA, HSA, or MSA binder). In some embodiments the serum albumin binder is an albumin binding peptide. Peptides that bind to albumin are described in WO200145746, WO2002076489, WO2008068280, WO2009127691, WO2011095545, and US Pat. Pub. Nos. 20040001827, 20080187517, and 20130316952. In some embodiments the albumin binding peptide comprises a sequence that is at least 90%, e.g., 100%, identical to DICLPRWGCLW (SEQ ID NO: 91). See Dennis M S, et al. (2002) Albumin binding as a general strategy for improving the pharmacokinetics of proteins. J Biol. Chem. 277: 35035-43. In some embodiments the albumin binding peptide comprises a sequence at least 90%, 95%, or more (e.g., 100%) identical to RLIEDICLPRWGCLWEDD (SEQ ID NO: 68) or QRLMEDICLPRWGCLWEDDF (SEQ ID NO: 69). In certain embodiments the albumin binding peptide comprises a sequence at least 90%, 95%, or more (e.g., 100%) identical to the sequence of the peptide known as ABD035, which is SDFYKRLINKAKTVEGVEALKLHILAALP (SEQ ID NO: 70). In some embodiments, a serum albumin binder is an HSA binding polypeptide. In some embodiments, an HSA binding polypeptide is a variant of Sac7d (SEQ ID NO: 1). In certain embodiments, a variant has between 13 and 17 substitutions as compared to Sac7d. A variant of Sac7d (SEQ ID NO: 1) may be represented as MVKVKFVFSGEEKEVXTSKIKWVLRWGKAVGFKYDDXGKIGYGFVAEKDAPKELLDX LARAEREKK (SEQ ID NO: 64), wherein each X independently represents an amino acid, wherein the polypeptide comprises: D or E at position 16; N or Q at position 37; and M or L at position 57. In some embodiments, an HSA binding polypeptide comprises D at position 16, N at position 37, and M at position 57 in SEQ ID NO: 64. In some embodiments, an HSA binding polypeptide comprises E at position 16, Q at position 37, and L at position 57 in SEQ ID NO: 64. As noted above, the methionine at the N-terminus is not necessary for HSA binding and may be omitted from any of the polypeptides described herein. One or more amino acids located at the C-terminus of the polypeptide (e.g., lysine) are not necessary for HSA binding and may be omitted. In some embodiments, an albumin binding polypeptide (e.g., HSA binding polypeptide) comprises an amino acid sequence of VKVKFVFSGEEKEVXTSKIKWVLRWGKAVGFKYDDXGKIGYGFVAEKDAPKELLDXL ARAEREK (SEQ ID NO: 71), wherein each X independently represents an amino acid, wherein the polypeptide comprises: D or E at position 15; N or Q at position 36; and M or L at position 56, and optionally where the peptide does not include the lysine at the C-terminal. In some embodiments, an albumin binding polypeptide (e.g., HSA binding polypeptide) comprises an amino acid sequence of VKVKFVFSGEEKEVDTSKIKWVLRWGKAVGFKYDDNGKIGYGFVAEKDAPKELLDML ARAEREK (SEQ ID NO: 72), optionally where the peptide does not include the lysine at the C-terminal. In some embodiments, an albumin binding polypeptide (e.g., HSA binding polypeptide) comprises an amino acid sequence of VKVKFVFSGEEKEVETSKIKWVLRWGKAVGFKYDDQGKIGYGFVAEKDAPKELLDLL ARAEREK (SEQ ID NO: 73), optionally where the peptide does not include the lysine at the C-terminal.

In some embodiments the heterologous polypeptide that extends the half-life comprises a hyaluronan binding domain. Hyaluronan (HA), also known as hyaluronic acid, is a glycosaminoglycan that is found in connective and other tissues and is abundant in synovial fluid, skin, and the vitreous body. HA binds to a large number naturally occurring hyaluronan-binding proteins (HABPs). Some HABPs contain an HA binding domain referred to as a link module through which they bind to HA (Kohda, C. J et al., Cell 86 (1996) 767-775.). Some HAPBs contain a linear 9-11 residue HA-binding motif containing multiple basic amino acids termed a B-X7-B motif (Yang B., et al. Identification of a common hyaluronan binding motif in the hyaluronan binding proteins RHAMM, CD44 and link protein. EMBO J., 13: 286-296, 1994). Each B in a B-X7-B motif is independently arginine or lysine, and each X is independently any amino acid other than aspartic acid or glutamic acid. In some embodiments at least one X is a basic amino acid (arginine, lysine, or histidine, typically arginine or lysine). HA binding peptides comprising a B-X7-B motif can be identified within HABP sequences or generated using methods such as phage display. RHAMM, IHABP, cdc37, P-32, SPACR and SPACRCAN are examples of HABPs that bind to HA via a B-X7-B motif. Examples of peptides comprising a B-X7-B motif are described in Amemiya, K., et al., (2005) Biochimica et Biophysica Acta 1724: 94-99). In some embodiments the HA binding peptide comprises the sequence RYPISRPRKR (SEQ ID NO: 74). In some embodiments the HA-binding peptide known as Pep-1 (GAHWQFNALTVR (SEQ ID NO: 75)) may be used. Additional HA binding peptides that may be used are described in WO2015009787. In some embodiments a HEP comprises an elastin-like polypeptide (ELP). The ELP sequence contains repeats of V-P-G-X-G, where X is any amino acid except proline (SEQ ID NO: 90). Exemplary ELPs are described in US Pat. Pub. No. 20110039776.

In some embodiments the HEP is a PAS polypeptide. As used herein a “PAS polypeptide” is a polypeptide characterized in that the sum of proline, alanine, and serine residues constitutes more than about 80%, or about 85%, or about 90%, or about 95%, or about 96%, or about 97%, or about 98%, or about 99%, or 100% of the total amino acid sequence of the HEP. In general, a PAS polypeptide is characterized in that it adopts a random coil conformation under physiological conditions as described in US Pat. Pub. No. 20100292130. PAS polypeptides that may be used as HEPs in the fusion proteins of the present disclosure are further described in WO 2008/155134, US Pat. Pub. No. 20100292130, U.S. Pat. No. 8,563,521 and/or U.S. Pat. No. 9,260,494.

In some embodiments a HEP consists solely of proline and alanine or consists predominantly of proline and alanine but can have up to 1%, 2%, 3%, 5%, or 10% other amino acid residues. Where other amino acids are present, they may all be the same, or multiple different amino acids may be present. Examples of polypeptides that are composed predominantly or entirely of proline and alanine and adopt a random coil conformation under physiological conditions, referred to as proline/alanine random coil polypeptides, are described in US Pat. Pub. No. 20130072420, U.S. Pat. Nos. 9,221,882, and/or U.S. Pat. No. 10,081,657.

In some embodiments a HEP is characterized in that the sum of glycine, alanine, serine, threonine, glutamate, and proline residues constitutes more than about 80%, or about 85%, or about 90%, or about 95%, or about 96%, or about 97%, or about 98%, or about 99%, or 100% of the total amino acid sequence of the HEP, and the HEP comprises at least 4 of these 6 different amino acids. Such a polypeptide may be referred to as being composed predominantly of amino acids selected from G, A, S, T, E, and P. A HEP composed predominantly of amino acids selected from G, A, S, T, E, and P may substantially lack secondary structure, e.g., may have no more than 5% of its amino acid residues participating in an alpha helix and no more than 5% 3-sheet structure, e.g., less than 2% alpha-helices and 2% beta-sheets, as determined by the Chou-Fasman algorithm (Chou, P. Y., et al. (1974) Biochemistry, 13: 222-45) and/or exhibit greater than 90% random coil formation as determined by the Garnier-Osguthorpe-Robson (“GOR”) algorithm (Gamier J, Gibrat J F, Robson B. (1996) Methods Enzymol 266:540-553), as described in US Pat. Pub. No. 20030228309. In some embodiments a HEP enriched for G, A, S, T, E, P and substantially lacking secondary structure comprises an XTEN polypeptide as described in Schellenberger et al., (2009) Nature Biotechnology, 27, 1186-90, WO2010091122, US Pat. Pub. Nos. 20100239554, 20170016042, U.S. Pat. Nos. 8,673,860; 8,703,717; 9,371,369; 9,926,351; 9,976,166; and/or 10,961,287.

In some embodiments a HEP may be up to about 1,000 amino acids long, e.g., between 100 and 250 amino acids long, between 250 and 500 amino acids long, or between 500 and 1000 amino acids long. In some embodiments a protein may comprise two or more HEPs, wherein the total number of amino acids in the HEPs is between 100 and 250, between 250 and 500, or between 500 and 1000. In some embodiments a HEP may be about 100, about 200, about 300, about 400, or about 500 amino acids long. In some embodiments a HEP may be about 600, about 700, about 800, about 900, or about 1000 amino acids long.

In some embodiments, the disclosure provides proteins (e.g., fusion proteins) that comprise a C3 and/or C3b binding polypeptide and two or more heterologous polypeptides. In some embodiments at least one of the heterologous polypeptides is an angiogenesis inhibitor, e.g., a VEGF inhibitor, e.g., a VEGF-inhibiting Fab such as ranibizumab. In some embodiments the disclosure provides proteins (e.g., fusion proteins) comprising a C3 and/or C3b binding polypeptide, an angiogenesis inhibitor and a polypeptide that extends the half-life of the protein such as those described above (e.g., an Fc domain, transferrin, albumin, an albumin fragment, an albumin binding peptide, or an HA binding peptide). Such proteins may have a variety of different designs. For example, a half-life extending polypeptide may be linked to an N-terminus or a C-terminus of a protein comprising a C3 and/or C3b binding polypeptide and a VEGF inhibitor. In some embodiments a half-life extending polypeptide may be linked to the N-terminus or the C-terminus of a linear polypeptide comprising a C3 and/or C3b binding polypeptide and a VEGF inhibitor. In some embodiments a modified Fab comprises a C3 and/or C3b binding polypeptide fused to the Fab at position A, B, C, or D (where positions are as shown in FIG. 1) and a heterologous polypeptide (e.g., a half-life extending polypeptide) fused to one of the other positions. For example, in some embodiments a modified Fab comprises a C3 and/or C3b binding polypeptide fused (e.g., directly or indirectly) to CH1 at position A and a heterologous polypeptide (e.g., a HEP) fused to CL at position C. In some embodiments a modified Fab comprises a C3 and/or C3b binding polypeptide fused to CL at position C (i.e., at the C-terminus of the light chain) and a heterologous polypeptide (e.g., a HEP) fused (e.g., directly or indirectly) to CH1 at position A (i.e., at the C-terminus of the heavy chain).

A protein comprising a Fab, one or more C3 and/or C3b binding polypeptides, and one or more half-life extending polypeptides may have a variety of different structural arrangements. In the representations below, NF represents a C3 and/or C3b binding polypeptide, HEP represents a half-life extending polypeptide, and HC and LC represent the heavy chain and the light chain of a Fab, respectively. The moieties present in each polypeptide chain are listed in an N-terminal to C-terminal order and a line (−) between two moieties indicates that the two moieties are fused to one another to form a single polypeptide chain either directly or via a peptide linker. The heavy and light chains are paired and joined to each other via a disulfide bond.

In some embodiments an NF and a HEP are fused to a HC, which is paired with a LC. A protein having this arrangement of moieties may be represented as follows: (1) HEP-HC NF paired with LC, (2) NF-HC-HEP paired with LC; (3) HC-HEP NF paired with LC; (4) HC-NF-HEP paired with LC; (5) HEP-NF-HC paired with LC; or (6) NF-HEP-HC paired with LC.

In some embodiments an NF and a HEP are fused to a LC, which is paired with a HC. A protein having this arrangement of moieties may be represented as follows: (7) HEP-LC NF paired with HC; (8) NF-LC-HEP paired with HC; (9) LC-HEP-NF paired with HC; (10) LC-NF-HEP paired with HC; (11) HEP-NF-LC paired with HC; (12) NF-HEP LC paired with HC.

In some embodiments an NF is fused to a HC, which is paired with a LC to which a half-life extending region is fused. A protein having this arrangement of moieties may be represented as follows: (13) NF-HC paired with LC-HEP; (14) NF-HC paired with HEP LC; (15) HC-NF paired with LC-HEP; or (16) HC-NF paired with HEP-LC.

In some embodiments an NF is fused to a LC, which is paired with a HC to which a half-life extending region is fused. A protein having this arrangement of moieties may be represented as follows: (17) NF-LC paired with HEP-HC; (18) LC-NF paired with HEP HC; (19) NF-LC paired with HC-HEP; (20) LC-NF paired with HC-HEP.

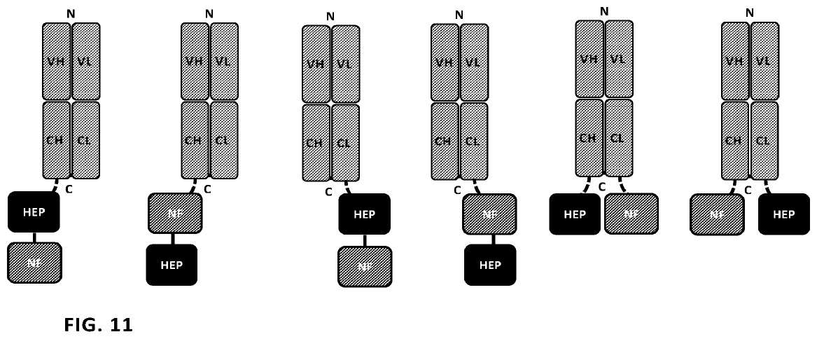

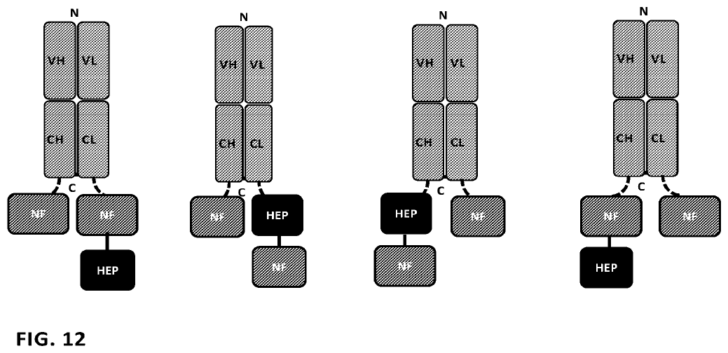

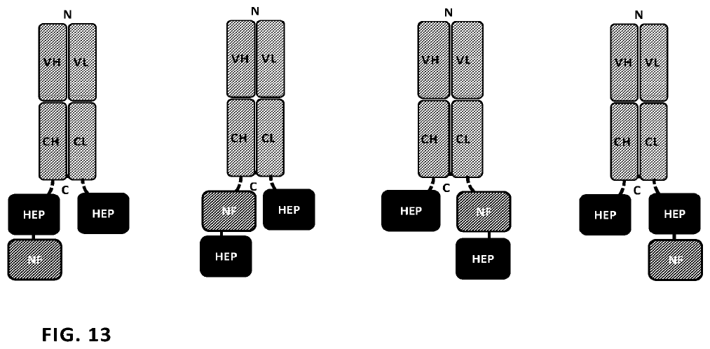

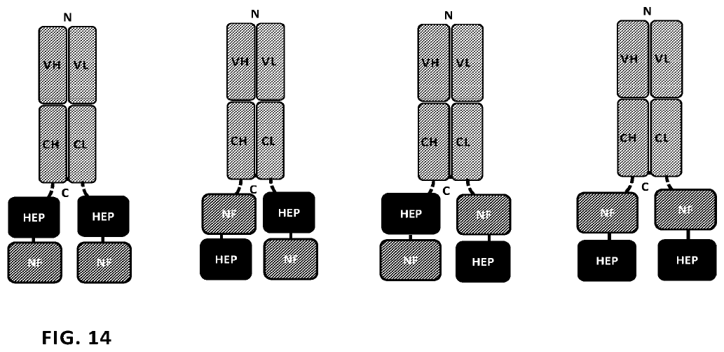

In some embodiments one or more NF(s) is/are fused to the C-terminal end of the heavy chain and/or the light chain of a Fab and one or more half-life extending region(s) is/are fused to the C-terminal end of the heavy chain and/or the light chain of the Fab, with the N-terminal ends of the heavy chain and the light chain not being fused to additional moieties. Proteins represented as (3), (4), (9), (10), (15), and (20) above are examples of these configurations. FIG. 11 provides schematic diagrams of certain of these proteins. FIGS. 12 and 13 provide schematic diagrams of proteins containing either two C3 and/or C3b binding polypeptides and one HEP or two HEPs and one C3 and/or C3b binding polypeptide. FIG. 14 provides schematic diagrams of proteins that contain two C3 and/or C3b binding polypeptides and two half-life extending regions. It is to be understood that fusion proteins of the disclosure can include fusions directly or indirectly to the CH or CL domain of the Fabs shown in FIGS. 10-14. For example, in some embodiments, the Fabs shown in FIGS. 10-14 include one or more amino acids between the CH domain and the NF or HEP. For example, a portion of a hinge region (e.g., EPKSCDKTH (SEQ ID NO: 44) or a portion thereof), optionally followed by a peptide linker, may be present between the CH domain and the NF or HEP. In some embodiments a sequence comprising or consisting of EPKSCDKTH (SEQ ID NO: 44) or a portion thereof or EPKSCDKTHL (SEQ ID NO: 67) or a portion thereof, optionally followed by a peptide linker, may be present between the CH domain and the NF or HEP. Many variations are possible. For example, each instance of HEP could represent two or more HEPs joined by peptide linkers. Similarly, each instance of “NF” could represent two or more C3 and/or C3b binding polypeptides joined by peptide linkers. In some embodiments any of the polypeptides in any of the structures depicted in FIGS. 10-14 may be extended to include one additional HEP. In some embodiments any of the polypeptides in any of the structures depicted in FIGS. 10-14 may be extended to include one additional C3 and/or C3b binding polypeptide. In some embodiments any of the polypeptides in any of the structures depicted in FIGS. 10-14 may be extended to include one additional HEP and one additional C3 and/or C3b binding polypeptide, which may be arranged in either order. In some embodiments any of the polypeptides in any of the structures depicted in FIGS. 10-14 may be extended to include one or more (e.g., two) additional HEP(s) and/or one or more (e.g., two) additional C3 and/or C3b binding polypeptide(s), which may be arranged in any order. It is to be understood that in any of the descriptions or depictions of a Fab herein (e.g., in FIG. 1, FIGS. 10-14), the heavy chain of the Fab may comprise a portion of a hinge region on the carboxyl-terminal end of the CH1 domain. While proteins comprising a Fab and a C3 and/or C3b binding polypeptide and, optionally, a HEP, are of particular interest herein, the disclosure provides analogous embodiments in which a different antibody fragment containing an antigen-binding domain, or a full size antibody, is used instead of a Fab.

In proteins that comprise two or more C3 and/or C3b binding polypeptides, the C3 and/or C3b binding polypeptides may be the same or different. It is particularly contemplated that they are the same. In proteins that comprise two or more HEPs, the HEPs may be the same or different. Peptide linkers may be present between any two of the regions in any of the proteins described herein. In proteins that comprise two or more peptide linkers, the peptide linkers may be the same or different, and any of the peptide linkers described herein may be used.

In some embodiments the modified Fab comprises a heavy chain having the amino acid sequence of SEQ ID NO: 36 and a light chain having the amino acid sequence of SEQ ID NO: 35, wherein a C3 and/or C3b binding polypeptide is fused at position A, B, C, or D, and a half-life extending polypeptide is fused at one of the other positions. In some embodiments a half-life extending polypeptide is fused to the N-terminus of SEQ ID NO: 37, SEQ ID NO: 39, or SEQ ID NO: 55 In some embodiments a half-life extending polypeptide is fused to the N-terminus of SEQ ID NO: 38, SEQ ID NO: 40, or SEQ ID NO: 56. In some embodiments a half-life extending polypeptide is fused to the C-terminus of SEQ ID NO: 37, SEQ ID NO: 39, or SEQ ID NO: 55. In some embodiments a half-life extending polypeptide is fused to the C-terminus of SEQ ID NO: 38, SEQ ID NO: 40, or SEQ ID NO: 56. In some embodiments a modified Fab comprises a light chain of a Fab (e.g., SEQ ID NO: 35) and a heavy chain of a Fab (e.g., SEQ ID NO: 36), wherein a HEP is fused to the C-terminus of the heavy chain and a C3 and/or C3b binding polypeptide (e.g., SEQ ID NO: 27, SEQ ID NO: 33, or SEQ ID NO: 34 or a C3 and/or C3b binding portion of SEQ ID NO: 27, SEQ ID NO: 33, or SEQ ID NO: 34) is fused to the C-terminus of the HEP. In some embodiments a modified Fab comprises a light chain of a Fab (e.g., SEQ ID NO: 35) and a heavy chain of a Fab (e.g., SEQ ID NO: 36), wherein a HEP is fused to the C-terminus of the light chain and a C3 and/or C3b binding polypeptide (e.g., SEQ ID NO: 27, SEQ ID NO: 33, or SEQ ID NO: 34 or a C3 and/or C3b binding portion of SEQ ID NO: 27, SEQ ID NO: 33, or SEQ ID NO: 34) is fused to the C-terminus of the HEP. Any C3 and/or C3b binding portion may be used. For example, in some embodiments amino acids 2-64 of SEQ ID NO: 27, SEQ ID NO: 33, or SEQ ID NO: 34 are used. In some embodiments amino acids 2-65 of SEQ ID NO: 27, SEQ ID NO: 33, or SEQ ID NO: 34 are used.

In some embodiments a modified Fab comprises SEQ ID NO: 35 and SEQ ID NO: 38 and further comprises a half-life extending polypeptide fused to the C-terminus of SEQ ID NO: 38. In some embodiments a modified Fab comprises SEQ ID NO: 35 and SEQ ID NO: 40 and further comprises a half-life extending polypeptide fused to the C-terminus of SEQ ID NO: 40. In some embodiments a modified Fab comprises SEQ ID NO: 35 and SEQ ID NO: 56 and further comprises a half-life extending polypeptide fused to the C-terminus of SEQ ID NO: 56. In some embodiments a modified Fab comprises SEQ ID NO: 36 and SEQ ID NO: 37 and further comprises a half-life extending polypeptide fused to the C-terminus of SEQ ID NO: 37. In some embodiments a modified Fab comprises SEQ ID NO: 36 and SEQ ID NO: 39 and further comprises a half-life extending polypeptide fused to the C-terminus of SEQ ID NO: 39. In some embodiments a modified Fab comprises SEQ ID NO: 36 and SEQ ID NO: 55 and further comprises a half-life extending polypeptide fused to the C-terminus of SEQ ID NO: 55. In some embodiments a modified Fab comprises SEQ ID NO: 35 and SEQ ID NO: 38 and further comprises a half-life extending polypeptide fused to the C-terminus of SEQ ID NO: 38. In some embodiments a modified Fab comprises SEQ ID NO: 35 and SEQ ID NO: 40 and further comprises a half-life extending polypeptide fused to the C-terminus of SEQ ID NO: 40. In some embodiments a modified Fab comprises SEQ ID NO: 35 and SEQ ID NO: 56 and further comprises a half-life extending polypeptide fused to the C-terminus of SEQ ID NO: 56. In some embodiments a modified Fab comprises SEQ ID NO: 36 and SEQ ID NO: 37 and further comprises a half-life extending polypeptide fused to the C-terminus of SEQ ID NO: 37. In some embodiments a modified Fab comprises SEQ ID NO: 36 and SEQ ID NO: 39 and further comprises a half-life extending polypeptide fused to the C-terminus of SEQ ID NO: 39. In some embodiments a modified Fab comprises SEQ ID NO: 36 and SEQ ID NO: 55 and further comprises a half-life extending polypeptide fused to the C-terminus of SEQ ID NO: 55. FIGS. 11, 12, 13, and 14 present schematic diagrams of proteins comprising a Fab, one or more C3 and/or C3b binding polypeptide(s), and one or more HEPs. Any of the C3 and/or C3b binding polypeptide(s) or light or heavy chain of a Fab may be joined to a half-life extending polypeptide by a peptide linker, such as those described herein (e.g., a 15 amino acid linker).

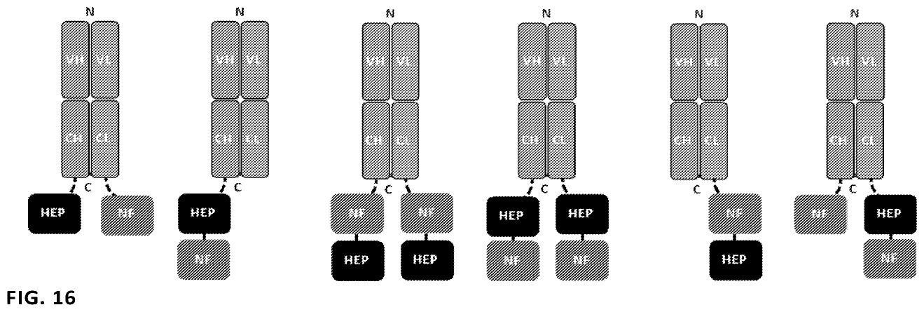

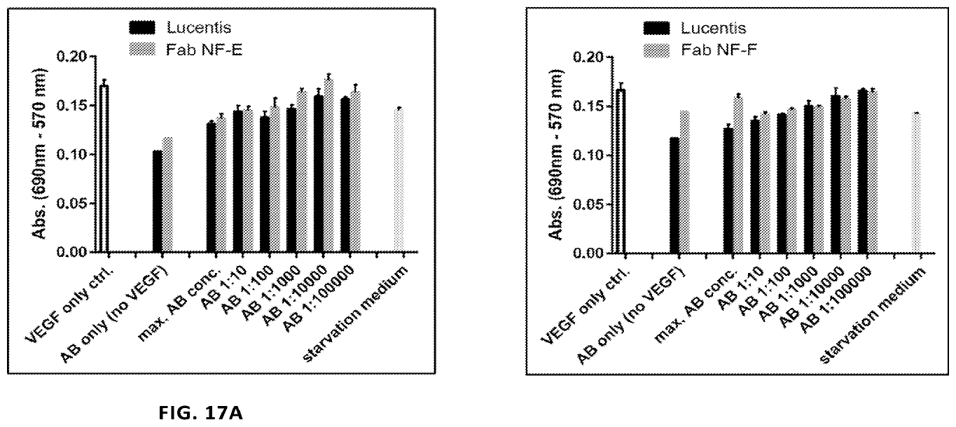

In some embodiments, a modified Fab comprises SEQ ID NO: 59 and SEQ ID NO: 57. In some embodiments, a modified Fab comprises SEQ ID NO: 63 and SEQ ID NO: 35. In some embodiments, a modified Fab comprises SEQ ID NO: 61 and SEQ ID NO: 60. In some embodiments, a modified Fab comprises SEQ ID NO: 63 and SEQ ID NO: 62. In some embodiments, a modified Fab comprises SEQ ID NO: 36 and SEQ ID NO: 60. In some embodiments, a modified Fab comprises SEQ ID NO: 58 and SEQ ID NO: 62. FIG. 16 presents schematic diagrams of proteins comprising a Fab, one or more C3 and/or C3b binding polypeptide(s), and one or more HEPs.

A variety of methods of making polypeptides are known in the art and can be used to make a C3 and/or C3b binding polypeptide or fusion protein or protein conjugate described herein. For example, a C3 and/or C3b binding polypeptide or fusion protein described herein can be recombinantly produced by utilizing a host cell system engineered to express a nucleic acid encoding a C3 and/or C3b binding polypeptide or fusion protein. Alternatively or additionally, a C3 and/or C3b binding polypeptide or fusion protein or protein conjugate described herein can be partially or fully prepared by chemical synthesis.

In order to produce a protein comprising a C3 and/or C3b binding polypeptide fused to a heterologous polypeptide, one can use a nucleic acid construct comprising (i) a DNA sequence comprising a sequence coding for a C3 and/or C3b binding polypeptide fused, at its 3′ end with a sequence coding for a heterologous polypeptide (e.g., a polypeptide comprising a VEGF inhibitor and/or a half-life extending polypeptide), optionally comprising an sequence coding for a linker located between these two sequences) or (ii) a DNA sequence comprising a sequence coding for a heterologous polypeptide (e.g., a polypeptide comprising a VEGF inhibitor and/or a half-life extending polypeptide) fused, at its 3′ end with a sequence coding for a C3 and/or C3b binding polypeptide, optionally comprising an sequence coding for a linker located between these two sequences.

In some embodiments a C3 and/or C3 binding polypeptide is fused to a heterologous polypeptide that comprises an antibody chain. Such a modified antibody chain may be joined to a second antibody chain via one or more disulfide bonds. In order to produce an antibody having a C3 and/or C3b binding polypeptide fused thereto, one can use a nucleic acid construct comprising a DNA sequence selected from the group consisting of: (i) a sequence coding for the heavy chain of an antibody fused, at its 3′ end with a sequence coding for the C3 and/or C3b binding polypeptide (optionally comprising a sequence coding for a linker located between these two sequences); (ii) a sequence coding for the heavy chain of an antibody fused, at its 5′ end with a sequence coding for the C3 and/or C3b binding polypeptide (optionally comprising a sequence coding for a linker located between these two sequences); (iii) a sequence coding for the light chain of an antibody fused, at its 3′ end with a sequence coding for the C3 and/or C3b binding polypeptide (optionally comprising a sequence coding for a linker located between these two sequences); (iv) a sequence coding for the light chain of an antibody fused, at its 5′ end with a sequence coding for the C3 and/or C3b binding polypeptide (optionally comprising a sequence coding for a linker located between these two sequences). A sequence coding for a further polypeptide, e.g., a half-life extending polypeptide, and, optionally, a linker peptide, may be incorporated into the construct in frame with any of the other coding sequences. In some embodiments a sequence encoding a HEP is located between a sequence encoding the heavy chain of a Fab and a sequence encoding a C3 and/or C3b binding polypeptide. In some embodiments a sequence encoding a HEP is located between a sequence encoding a light chain of a Fab and a sequence encoding a C3 and/or C3b binding polypeptide.

Fusion of a C3 and/or C3b binding polypeptide can be at the N-terminus and/or the C-terminus of the antibody chain (heavy and/or light chain). In certain embodiments the antibody may be a Fab. A molecule having the structure of a Fab and further binding region(s) comprising one or more C3 and/or C3b binding polypeptide(s) may be produced by co-expressing a heavy chain and a light chain of a Fab, wherein one or both of the chains is fused to a C3 and/or C3b binding polypeptide and/or to a half-life extending polypeptide, as described above. The heavy and light chains may be joined via a disulfide bond. In certain embodiments the antibody may be a full size antibody. A molecule having the structure of a full size antibody (two light chains paired to two heavy chains, and such dimers paired together), and further binding region(s) comprising one or more C3 and/or C3b binding polypeptide(s) can be produced by co-expressing heavy chain and a light chain of an antibody, wherein one or both of the chains is fused to a C3 and/or C3b binding polypeptide as described above. Each heavy chain is paired to a light chain via a disulfide bonds and two heavy chains (each paired to a light chain) are connected in the hinge region via disulfide bond(s),

Where a C3 and/or C3b binding polypeptide or fusion protein is recombinantly produced, any expression system can be used. Known expression systems include, without limitation, for example, egg, baculovirus, plant, yeast, or mammalian cells. In some embodiments, a C3 and/or C3b binding polypeptide or fusion protein described herein suitable for use in methods described herein are produced in mammalian cells. Non-limiting examples of mammalian cells that can be used include BALB/c mouse myeloma line (NSO/l, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); human fibrosarcoma cell line (e.g., HT1080); baby hamster kidney cells (BHK, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

The nucleic acid construct(s) encoding the C3 and/or C3b binding protein or encoding fusion protein(s) of interest (e.g., antibody chain fused to a C3 and/or C3b binding polypeptide) may comprise a promoter operably linked to the coding sequence. The nucleic acid construct(s) may be introduced into cells in order to produce the protein. The protein may be harvested from the cells or secreted into culture medium and harvested from the culture medium.

### IV. COMPOSITIONS AND ADMINISTRATION

C3 and/or C3b binding polypeptides described herein can be used to treat a complement-mediated disease or disorder, e.g., in subjects suffering from or susceptible to a complement-mediated disease or disorder described herein. It is noted that wherever the present disclosure refers to uses (e.g., methods of treatment), activity, methods of measuring activity, methods of making, and/or compositions (e.g., pharmaceutical compositions), comprising or using or otherwise relating to a C3 and/or C3b binding polypeptide, such disclosure encompasses embodiments in which any of the agents disclosed herein comprising a C3 and/or C3b polypeptide may be used, e.g., embodiments in which a C3 and/or C3b binding polypeptide is fused, conjugated, or otherwise linked to one or more heterologous polypeptide(s) (e.g., a VEGF inhibitor, e.g., a polypeptide chain of a VEGF inhibiting Fab) and embodiments in which a C3 and/or C3b binding polypeptide is not fused, conjugated, or otherwise linked to a heterologous polypeptide. For example, in certain embodiments, a protein comprising a C3 and/or C3b binding polypeptide and a heterologous polypeptide (e.g., a VEGF inhibitor), a protein comprising a C3 and/or C3b binding polypeptide and a half-life extending polypeptide, and/or a protein comprising a C3 and/or C3b binding polypeptide and a VEGF inhibitor and a half-life extending polypeptide may be used to treat any complement-mediated disease or disorder described herein. In certain embodiments a modified VEGF-inhibiting Fab comprising a heavy chain and a light chain, at least one of which is fused to a C3 and/or C3b binding polypeptide may be used to treat any complement-mediated disease or disorder described herein.

In some embodiments a protein comprising a C3 and/or C3b binding polypeptide and a VEGF inhibitor may be used to treat a subject suffering from or at risk of a complement-mediated disorder that is characterized by pathologic angiogenesis and/or pathologic blood vessel permeability or is associated with increased risk of developing pathologic angiogenesis and/or pathologic blood vessel permeability. In some embodiments the protein is a modified VEGF-inhibiting Fab having a C3 and/or C3b binding polypeptide fused to a heavy chain or a light chain of the Fab, as described herein. Pathologic angiogenesis and/or pathologic blood vessel permeability refers to angiogenesis or blood vessel permeability that is aberrant, excessive, and/or otherwise detrimental to a subject who experiences it. In some embodiments the disorder is an eye disorder. In some embodiments the eye disorder is AMD. In some embodiments the protein is administered to an eye suffering from GA, wherein the eye has not been diagnosed with neovascular AMD. In some embodiments the protein is administered to an eye suffering from neovascular AMD, wherein the eye has not been diagnosed with GA. In some embodiments the protein is administered to an eye suffering from neovascular AMD and GA. In some embodiments the protein is administered to an eye suffering from neovascular AMD and intermediate AMD. In some embodiments the protein is administered to an eye suffering from neovascular AMD, wherein the eye has not been diagnosed with GA. In some embodiments the eye disorder is choroidal neovascularization (CNV), which may be a manifestation of AMD or may result from other causes. In some embodiments the eye disorder is proliferative diabetic retinopathy, neovascular glaucoma, diabetic macular edema, retinopathy of prematurity, or macular edema secondary to retinal vein occlusions. In some embodiments the disorder is cancer.

One with skill in the art, e.g., a physician, is aware that dosage regimens can be adjusted to provide the desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. The mode of administration is left to the discretion of the practitioner.

In some embodiments a C3 and/or C3b binding polypeptide (or a protein comprising a C3 and/or C3b binding polypeptide) may be administered systemically, e.g., intravenously or subcutaneously, for treatment of a complement-mediated disorder described herein. In some embodiments a local administration route may be used, e.g., where the disorder primarily affects a particular body system, organ, or tissue. For example, in some embodiments a C3 and/or C3b binding polypeptide may be administered intraocularly (e.g., intravitreally) for treating an eye disorder. In some embodiments a C3 and/or C3b binding polypeptide is administered by the pulmonary route (e.g., for treating a disorder affecting the respiratory system). In some embodiments, a C3 and/or C3b binding polypeptide is administered intrathecally for treatment of a complement-mediated disorder that affects the central nervous system as described herein. In some embodiments intracisternal or intracerebroventricular administration may be used for treatment of a complement-mediated disorder that affects the central nervous system. Routes of administration described herein may be used to administer C3 and/or C3b binding polypeptides or nucleic acids or gene therapy vectors encoding such proteins.

C3 and/or C3b binding polypeptides (or proteins comprising C3 and/or C3b binding polypeptides) can be incorporated into pharmaceutical compositions. Such pharmaceutical compositions are useful for, among other things, administration and delivery to a subject in vivo. In some embodiments, pharmaceutical compositions also contain a pharmaceutically acceptable carrier or excipient. Such excipients include any pharmaceutical agent, e.g., a pharmaceutical agent that does not itself induce an immune response harmful to the individual receiving the composition, and which may be administered without undue toxicity. As used herein the terms “pharmaceutically acceptable” and “physiologically acceptable” mean a biologically acceptable formulation, gaseous, liquid or solid, or mixture thereof, which is suitable for one or more routes of administration, in vivo delivery or contact. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

In some embodiments, a C3 and/or C3b binding polypeptide (or a protein comprising a C3 and/or C3b binding polypeptide) is prepared in a delivery system, such as a formulation that will protect the C3 and/or C3b binding polypeptide against rapid elimination or degradation in vivo. In some embodiments, a C3 and/or C3b binding polypeptide is incorporated into or encapsulated in a microparticle or nanoparticle formulation. Exemplary biodegradable and/or biocompatible polymers that may be used in a microparticle or nanoparticle formulation for a C3 and/or C3b binding polypeptide include, but are not limited to, one or more of ethylene vinyl acetate, polyanhydrides, polyglycolic acid, collagen, polyorthoesters, polyethers, polylactic acid, or poly(lactic-co-glycolic acid (PLGA). In some embodiments, a C3 and/or C3b binding polypeptide is incorporated into or encapsulated in a liposome or lipid-based particle. Liposomes or lipid-based particles for delivery of the C3 and/or C3b binding polypeptides or nucleic acids encoding such polypeptides can be prepared according to methods known to those skilled in the art, for example, as described in U.S. Pat. No. 4,522,811. In some embodiments, the lipid-based particle is a lipid nanoparticle.

Pharmaceutical compositions may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

Compositions of a C3 and/or C3b binding polypeptide (or a protein comprising a C3 and/or C3b binding polypeptide) described herein can be administered in a sufficient or effective amount to a subject in need thereof. Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in milligrams per kilogram of body weight (mg/kg) or vector genomes/per kilogram of body weight (vg/kg), will vary based on several factors including, but not limited to: route of administration, the level of C3 and/or C3b binding required to achieve a therapeutic effect, the specific disease treated, any host immune response to the C3 and/or C3b binding polypeptide, and the stability of the C3 and/or C3b binding polypeptide. One skilled in the art can determine a C3 and/or C3b binding polypeptide dose range to treat a patient having a particular disease or disorder based on the aforementioned factors, as well as other factors. Generally, doses will range from at least 0.002 mg/kg to about 50 mg/kg, such as about 0.001 mg/kg to about 10 mg/kg of the weight of the subject, to achieve a therapeutic effect.

In some embodiments a protein described herein comprising a C3 and/or C3b binding polypeptide and a VEGF inhibitor, e.g., a protein comprising one or more C3 and/or C3b binding polypeptides (e.g., two C3 and/or C3b binding polypeptides) fused to a Fab and optionally comprising one or more half-life extending polypeptides (e.g., two HEPs), is administered via intravitreal (IVT) administration at a dose between about 0.1 milligram (mg) to about 20 mg. In certain embodiments the dose is between about 0.1 mg and about 0.5 mg, between about 0.5 mg and about 1.0 mg, between about 1.0 mg and about 2.5 mg, between about 2.5 mg and about 5.0 mg, between about 5.0 mg and about 1.0 mg, or between about 10 mg and about 20 mg. In some embodiments a dose administered by IVT administration is administered in a volume of between about 25 microliters and about 50 microliters, between about 50 microliters and about 100 microliters, or between about 100 microliters and about 125 microliters, e.g., about 50 microliters, about 75 microliters, about 100 microliters, or about 125 microliters. In certain embodiments the dose of a protein having a structure as depicted in FIG. 14 is 1 mg to 2 mg. In some embodiments such dose is administered by IVT administration in a 50 microliter volume.

An effective amount or a sufficient amount can (but need not) be provided in a single administration, may require multiple administrations, and can be administered alone or in combination with another composition (e.g., another complement inhibitor described herein). For example, the amount may be proportionally increased as indicated by the need of the subject, type, status and severity of the disease treated or side effects (if any) of treatment. Amounts considered effective also include amounts that result in a reduction of the use of another treatment, therapeutic regimen or protocol, such as administration of another complement inhibitor described herein.

Accordingly, pharmaceutical compositions of the disclosure include compositions wherein the active ingredients are contained in an effective amount to achieve the intended therapeutic purpose. Determining a therapeutically effective dose is well within the capability of a skilled medical practitioner using the techniques and guidance provided in the disclosure. Therapeutic doses can depend on, among other factors, the age and general condition of the subject, the severity of the complement-mediated disease or disorder, and the binding affinity of the C3 and/or C3b binding polypeptide described herein. Thus, a therapeutically effective amount in humans will fall in a relatively broad range that may be determined by a medical practitioner based on the response of an individual patient to treatment. In some embodiments, a pharmaceutical composition comprises a C3 and/or C3b binding polypeptide (or a protein comprising a C3 and/or C3b binding polypeptide). In some embodiments, a pharmaceutical composition comprises a nucleic acid encoding a C3 and/or C3b binding polypeptide (or a protein comprising a C3 and/or C3b binding polypeptide). In some embodiments, a pharmaceutical composition comprises a vector, e.g., a viral vector, comprising a nucleic acid encoding a C3 and/or C3b binding polypeptide (or a protein comprising a C3 and/or C3b binding polypeptide) described herein. In some embodiments, a pharmaceutical composition comprises a cell expressing a C3 and/or C3b binding polypeptide (or a protein comprising a C3 and/or C3b binding polypeptide) described herein. In some embodiments, pharmaceutical compositions may be delivered to a subject, so as to allow production of an C3 and/or C3b binding polypeptide described herein in vivo by gene- and or cell-based therapies or by ex-vivo modification of the patient's or donor's cells which are then administered to the patient.

Methods and uses of the disclosure include delivery and administration systemically, regionally, or locally, by any route, for example, by injection or infusion. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in the treatment of patients with complement-mediated disorders may determine the optimal route for administration of polypeptides described herein.

In some embodiments, a C3 and/or C3b binding polypeptide (or a protein comprising a C3 and/or C3b binding polypeptide) may be administered to a subject once daily, once weekly, once every 2, 3, or 4 weeks, monthly, every other month, every 3 months, every 4 months, or even at longer intervals. In some embodiments, a subject is monitored before and/or following treatment for level of complement activity, e.g., as measured using an alternative pathway assay, a classical pathway assay, or both. Suitable assays are known in the art and include, e.g., a hemolysis assay. In some embodiments, a subject is treated, or is retreated, if a measured level of complement activity is more than 10%, 20%, 30%, 40%, 50%, 100%, 200%, or more, relative to measured level of complement activity in a control subject.

In some embodiments it is contemplated that a relatively short course of a C3 and/or C3b binding polypeptide (or a protein comprising a C3 and/or C3b binding polypeptide), alone or in combination with one or more additional complement inhibitors described herein, e.g., between 1 week and 6 weeks, e.g., about 2-4 week, may provide a long-lasting benefit. In some embodiments, a remission is achieved for a prolonged period of time, e.g., 1-3 months, 3-6 months, 6-12 months, 12-24 months, or more. In some embodiments a subject may be monitored and/or treated prophylactically before recurrence of symptoms. For example, a subject may be treated prior to or upon exposure to a triggering event. In some embodiments a subject may be monitored, e g., for an increase in complement activation and/or for return or worsening of one or more signs or symptoms of the disorder and may be treated upon such occurrence.

In some embodiments, a C3 and/or C3b binding polypeptide (or protein comprising a C3 and/or C3b binding polypeptide) described herein is delivered to a subject (e.g., to a cell of a subject, e.g., a liver cell of a subject or a cell in the eye of a subject such as a retinal pigment epithelial cell) using an expression vector. Many forms of vectors can be used to deliver a C3 and/or C3b binding polypeptide described herein. Non-limiting examples of expression vectors include viral vectors (e.g., vectors suitable for gene therapy), plasmid vectors, bacteriophage vectors, cosmids, phagemids, artificial chromosomes, and the like.

In some embodiments, a nucleotide sequence encoding a C3 and/or C3b binding polypeptide (or protein comprising a C3 and/or C3b binding polypeptide) described herein is integrated into a viral vector. Non-limiting examples of viral vectors include: retrovirus (e.g., Moloney murine leukemia virus (MMLV), Harvey murine sarcoma virus, murine mammary tumor virus, Rous sarcoma virus), adenovirus, adeno-associated virus, SV40-type virus, polyomavirus, Epstein-Barr virus, papilloma virus, herpes virus, vaccinia virus, and polio virus.

In some embodiments, hepatocytes are targeted for delivery of a C3 and/or C3b binding polypeptide (or protein comprising a C3 and/or C3b binding polypeptide) described herein. Several classes of viral vectors have been shown competent for liver-targeted delivery of a gene therapy construct, including retroviral vectors (see, e.g., Axelrod et al., PNAS 87:5173-5177 (1990); Kay et al., Hum. Gene Ther. 3:641-647 (1992); Van den Driessche et al., PNAS 96:10379-10384 (1999); Xu et al., ASAIO J. 49:407-416 (2003); and Xu et al., PNAS 102:6080-6085 (2005)), lentiviral vectors (see, e.g., McKay et al., Curr. Pharm. Des. 17:2528-2541 (2011); Brown et al., Blood 109:2797-2805 (2007); and Matrai et al., Hepatology 53:1696-1707 (2011)), adeno-associated viral (AAV) vectors (see, e.g., Herzog et al., Blood 91:4600-4607 (1998)), and adenoviral vectors (see, e.g., Brown et al., Blood 103:804-810 (2004) and Ehrhardt et al., Blood 99:3923-3930 (2002)).

Retroviruses are enveloped viruses that belong to the viral family Retroviridae. Protocols for the production of replication-deficient retroviruses are known in the art (see, e.g., Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991)). The recombinant virus can then be isolated and delivered to cells of the subject either in vivo or ex vivo. A number of retroviral systems are known in the art, for example See U.S. Pat. Nos. 5,994,136, 6,165,782, and 6,428,953.

In some embodiments, the retrovirus is a lentivirus of the Retroviridae family. In some examples, the lentivirus is, but is not limited to, human immunodeficiency viruses (HIV-1 and HIV-2), simian immunodeficiency virus (S1V), feline immunodeficiency virus (FIV), equine infections anemia (EIA), and visna virus.

In some embodiments, the vector is an adenovirus vector. The adenovirus on which a viral vector may be based may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087. Further examples of adenoviral vectors can be found in U.S. Publication Nos. 20150093831, 20140248305, 20120283318, 20100008889, 20090175897 and 20090088398.

In some embodiments, the viral vector is an adeno-associated virus (AAV) vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012)). Methods for generating and using recombinant AAV (rAAV) vectors are described, for example, in U.S. Pat. Nos. 5,139,941 and 4,797,368.

Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. In some embodiments, an AAV vector is an AAV2/6, AAV2/8 or AAV2/9 vector (e.g., AAV6, AAV8 or AAV9 serotype having AAV2 ITR). Other AAV vectors are described in, e.g., Sharma et al., Brain Res Bull. 2010 Feb. 15; 81(2-3): 273. In some embodiments, an AAV vector is a self-complementary AAV vector.

In some embodiments, an AAV vector is a naturally occurring AAV. In some embodiments, an AAV vector is a modified AAV (i.e., a variant of a naturally occurring AAV). In some embodiments, an AAV vector may be generated by directed evolution, e.g., by DNA shuffling, peptide insertion, or random mutagenesis, in order to introduce modifications into the AAV sequence to improve one or more properties for gene therapy, e.g., to avoid or lessen an immune response or recognition by neutralizing antibodies, and/or for more efficient and/or targeted transduction (Asuri et al., Molecular Therapy 20.2 (2012): 329-338). Methods of using directed evolution to engineer an AAV vector can be found, e.g., in U.S. Pat. No. 8,632,764. In some embodiments the modified AAV is modified to include a specific tropism.

The AAV sequences of a rAAV vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). An example of an rAAV vector of the present disclosure is a “cis-acting” plasmid containing the transgene (e.g., nucleic acid encoding an miRNA described herein), in which the selected transgene sequence and associated regulatory elements are flanked by the 5′ and 3′ AAV ITR sequences. The AAV ITR sequences may be obtained from any known AAV, including presently identified mammalian AAV types. In some embodiments, an AAV vector may be a dual or triple AAV vector, e.g., for the delivery of large transgenes (e.g., transgenes of greater than approximately 5kb). In some embodiments, a dual AAV vector may include two separate AAV vectors, each including a fragment of the full sequence of the large transgene of interest, and when recombined, the fragments form the full sequence of the large transgene of interest, or a functional portion thereof. In some embodiments, a triple AAV vector may include three separate AAV vectors, each including a fragment of the sequence of the large transgene of interest, and when recombined, the fragments form the full sequence of the large transgene of interest, or a functional portion thereof. The multiple AAV vectors of the dual or triple AAV vectors can be delivered to and co-transduced into the same cell, where the two or three fragments of transgene recombine together and generate a single mRNA transcript of the entire large transgene of interest. In some embodiments, the fragmented transgenes include non-overlapping sequences. In some embodiments, the fragmented transgenes include overlapping sequences.

In some embodiments, the multiple AAV vectors of the dual or triple AAV vector may be the same type of AAV vector. In some embodiments, the multiple AAV vectors of the dual or triple AAV vector may be different types of AAV vector.

A viral vector may also be based on an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819; the vectors and methods of their making are incorporated herein by reference in their entirety.

In addition to the major elements identified above for an AAV vector, the vector can also include conventional control elements operably linked to the transgene in a manner that permits its transcription, translation and/or expression in a cell transfected with the vector or infected with the virus produced by the disclosure. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters that are native, constitutive, inducible and/or tissue-specific, are known in the art and may be included in a vector described herein. In some embodiments, operably linked coding sequences yield a functional RNA.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, and the dihydrofolate reductase promoter. Inducible promoters and inducible systems are available from a variety of commercial sources, including, without limitation, Invitrogen and Clontech. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system.

In some embodiments, regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken 3-actin promoter, a pol II promoter, or a pol III promoter.

In some embodiments, a viral vector is designed for expressing a C3 and/or C3b binding polypeptide (or protein comprising a C3 and/or C3b binding polypeptide) in hepatocytes, and the viral vector (e.g., an AAV vector) includes one or more liver-specific regulatory elements, which substantially limit expression of the C3 and/or C3b binding polypeptide (or protein comprising the C3 and/or C3b binding polypeptide) to hepatic cells. Generally, liver-specific regulatory elements can be derived from any gene known to be exclusively expressed in the liver. WO 2009/130208 identifies several genes expressed in a liver-specific fashion, including serpin peptidase inhibitor, clade A member 1, also known as α-antitrypsin (SERPINA1; GeneID 5265), apolipoprotein C-I (APOC1; GeneID 341), apolipoprotein C-IV (APOC4; GeneID 346), apolipoprotein H (APOH; GeneID 350), transthyretin (TTR; GeneID 7276), albumin (ALB; GeneID 213), aldolase B (ALDOB; GeneID 229), cytochrome P450, family 2, subfamily E, polypeptide 1 (CYP2E1; GeneID 1571), fibrinogen alpha chain (FGA; GeneID 2243), transferrin (TF; GeneID 7018), and haptoglobin related protein (HPR; GeneID 3250). In some embodiments, a viral vector described herein includes a liver-specific regulatory element derived from the genomic loci of one or more of these proteins. In some embodiments, a promoter may be the liver-specific promoter thyroxin binding globulin (TBG). Alternatively, other liver-specific promoters may be used (see, e.g., The Liver Specific Gene Promoter Database, Cold Spring Harbor, http://rulai.cshl.edu/LSPD/, such as, e.g., alpha 1 anti-trypsin (A1AT); human albumin (Miyatake et al., J. Virol. 71:5124 32 (1997)); humA1b; hepatitis B virus core promoter (Sandig et al., Gene Ther. 3:1002 9 (1996)); or LSP1. Additional vectors and regulatory elements are described in, e.g., Baruteau et al., J. Inherit. Metab. Dis. 40:497-517 (2017)).

### V. MEASURING INTERACTIONS AND/OR ACTIVITY OF C3 AND/OR C3B BINDING POLYPEPTIDES

The ability of a C3 and/or C3b binding polypeptide (e.g., a monomer, dimer, fusion protein, or protein comprising a C3 and/or C3b binding polypeptide) described herein to bind to C3 and/or C3b can be measured by methods known in the art, e.g., any of the following methods: surface plasmon resonance (e.g., BIACORE analysis), isothermal titration calorimetry, enzyme linked immunosorbent assay (ELISA), X-ray crystallography, sequence analysis and scanning mutagenesis. For example, the binding interaction of a polypeptide and C3 and/or C3b can be analyzed using surface plasmon resonance (SPR). SPR or biomolecular interaction analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of a binding moiety to a target. Such data can be used to compare different molecules. Information from SPR can also be used to develop structure-activity relationships (SAR). For example, the kinetic and equilibrium binding parameters of particular binding moieties to targets at various pH levels can be evaluated. Variant amino acids at given positions can be identified that correlate with particular binding parameters, e.g., high affinity, low affinity, and low K_{off}, at particular pH levels. The K_{D} of a polypeptide binding to C3 and/or C3b may alternately or additionally be measured using isothermal titration calorimetry. In some embodiments, binding affinity of a C3 and/or C3b binding polypeptide may be correlated with activity, with a lower K_{D} indicating a higher binding affinity, as recognized in the art.

In some embodiments, a C3 and/or C3b binding polypeptide (or a protein comprising a C3 and/or C3b binding polypeptide) has a K_{D} of about 50 nM to about 60 nM, about 40 nM to about 50 nM, about 30 nM to about 40 nM, about 20 nM to about 30 nM, about 10 nM to about 20 nM, about 5 nM to about 10 nM, about 1 nM to about 5 nM, about 1 nM to about 2 nM, or about 0.1 nM to about 1 nM for binding to C3. In some embodiments, a C3 and/or C3b binding polypeptide (or protein comprising a C3 and/or C3b binding polypeptide) has a K_{D} of about 50 nM to about 60 nM, about 40 nM to about 50 nM, about 30 nM to about 40 nM, about 20 nM to about 30 nM, about 10 nM to about 20 nM, about 5 nM to about 10 nM, about 1 nM to about 5 nM, about 1 nM to about 2 nM, or about 0.1 nM to about 1 nM for binding to C3b. In some embodiments, a C3 and/or C3b binding polypeptide (or protein comprising a C3 and/or C3b binding polypeptide) has a K_{D} of between about 0.1 nM and about 1.0 nM for binding to C3 and for binding to C3b. In some embodiments, a C3 and/or C3b binding polypeptide (or protein comprising a C3 and/or C3b binding polypeptide) has a K_{D} of between about 0.1 nM and about 5.0 nM for binding to C3 and for binding to C3b. In some embodiments, a C3 and/or C3b binding polypeptide (or protein comprising a C3 and/or C3b binding polypeptide) has a K_{D} of between about 0.1 nM and about 10.0 nM for binding to C3 and for binding to C3b. In some embodiments, a C3 and/or C3b binding polypeptide (or protein comprising a C3 and/or C3b binding polypeptide) has a K_{D} of between about 1.0 nM and about 10.0 nM for binding to C3 and for binding to C3b.

The activity of a C3 and/or C3b binding polypeptide (or protein comprising a C3 and/or C3b binding polypeptide) may be expressed in terms of its IC₅₀ (the concentration of the compound that inhibits complement activation by 50%), with a lower IC₅₀ indicating a higher activity as recognized in the art. Complement activation may be measured using any suitable assay. In some embodiments a hemolysis assay may be used. In some embodiments the assay is a CH50 assay. In some embodiments the assay is an AH50 assay. some embodiments an ELISA assay may be used. The activity of a preferred C3 and/or C3b binding polypeptide (or protein comprising a C3 and/or C3b binding polypeptide) for use as described herein is greater than a Nanofitin control polypeptide (e.g., Sac7d wild-type protein). It will be appreciated that the precise IC₅₀ value measured for a given C3 and/or C3b binding polypeptide will vary with experimental conditions (e.g., the serum concentration used in the assay). Comparative values, e.g., obtained from experiments in which IC₅₀ is determined for multiple different compounds under substantially identical conditions, are of use. In some embodiments, a C3 and/or C3b binding polypeptide inhibits classical and/or alternative pathway complement activation with an IC₅₀ of 10 nm-100 nM, 100 nM-250 nM, 250 nM-500 nM, or 500 nM-1,000 nM. In some embodiments, a C3 and/or C3b binding polypeptide inhibits classical and/or alternative pathway complement activation with an activity at least 50%, 60%, 70%, 80%, 90%, or more of that of a compstatin analog of FIG. 8 on a molar basis.

### VI. DISEASES, DISORDERS, AND CONDITIONS

In some embodiments, a C3 and/or C3b binding polypeptide (or a protein comprising a C3 and/or C3b binding protein) described herein is administered to a subject suffering from or at risk of complement-mediated damage to an organ, tissue, or cells. In some embodiments, a C3 and/or C3b binding polypeptide (or a protein comprising a C3 and/or C3b binding polypeptide) described herein is administered in combination with one or more additional complement inhibitors to a subject suffering from or at risk of complement-mediated damage to an organ, tissue, or cells. In some embodiments, a C3 and/or C3b binding polypeptide (or a protein comprising a C3 and/or C3b binding polypeptide) described herein is contacted with an organ, tissue, or cells ex vivo. The organ, tissue, or cells can be introduced into a subject and can be protected from damage that would otherwise be caused by the recipient's complement system.

The beneficial effects of inhibiting complement activation at the surface of cells or other body structures can include protection of the cells or structures themselves against direct complement-mediated damage (e.g., preventing cell lysis). In some aspects, administration of a C3 and/or C3b binding polypeptide (or a protein comprising a C3 and/or C3b binding polypeptide) to a subject reduces deposition of C3 fragments on cells and/or cell structures such as synapses in the subject, thereby reducing the susceptibility of such cells or cell structures to phagocytosis. Inhibiting complement activation may have a variety of other beneficial effects. For example, inhibiting complement activation may reduce the generation of anaphylotoxins and resulting influx/activation of neutrophils and other pro-inflammatory events and/or reduce potentially damaging release of intracellular contents, thereby potentially having beneficial effects on remote organ systems or throughout the body.

In some embodiments, a C3 and/or C3b binding polypeptide (or a protein comprising a C3 and/or C3b binding polypeptide) disclosed herein inhibits cleavage of C3 (and/or reduces the level of a C3 cleavage product, e.g., C3a and/or C3b in serum). In some embodiments, a C3 and/or C3b binding polypeptide (or a protein comprising a C3 and/or C3b binding polypeptide) disclosed herein inhibits cleavage of C3 (and/or reduces the level of a C3 cleavage product, e.g., C3a and/or C5b in serum) as compared to a prior measurement from the same subject or a reference value. In some embodiments, administration of a C3 and/or C3b binding polypeptide (or a protein comprising a C3 and/or C3b binding polypeptide) as disclosed herein decreases the level or amount of C3 cleavage (and/or level or amount of C3a and/or level or amount of C3b) more than administration of a comparable amount (e.g., on a molar basis) of a reference polypeptide, e.g., Sac7d (SEQ ID NO: 1) or a control Nanofitin. In some embodiments, administration of a C3 and/or C3b binding polypeptide (or a protein comprising a C3 and/or C3b binding polypeptide) as disclosed herein decreases the level or amount of C3b (or a C3b cleavage product such as iC3b or C3d) deposited on cells of interest or cell structures (e.g., cells or cell structures susceptible to complement-mediated damage). In certain embodiments, administration of a C3 and/or C3b binding polypeptide (or a protein comprising a C3 and/or C3b binding polypeptide) as disclosed herein decreases deposition of C3b and/or C3b cleavage products on cells or cell structures of interest and thereby inhibits complement-mediated phagocytosis of cells or cell structures of interest. In some embodiments, cell structures of interest are synapses, e.g., in the central nervous system. In some embodiments, an inhibition or decrease is a reduction by at least about 10%, 20%, 30%, 40%, 50%, 60%, 70%, 80%, 90%, or 100%, as compared to a reference value. In some embodiments a reference value is a value measured in the same subject (or in a sample obtained from the subject) prior to administration of a C3 and/or C3b binding polypeptide (or a protein comprising a C3 and/or C3b binding polypeptide). In some embodiments a reference value may be a value obtained following administration of a comparable amount of a control polypeptide, e.g., Sac7d, or a control Nanofitin that does not detectably bind to C3 and/or C3b.

C3 and/or C3b binding polypeptides (or proteins comprising a C3 and/or C3b binding polypeptide) described herein are useful for preventing or treating various disorders or diseases. In some embodiments, the present disclosure provides methods for treating a complement-mediated disorder comprising administering to a subject susceptible to such disorder an effective amount of a C3 and/or C3b binding polypeptide (or a protein comprising a C3 and/or C3b binding polypeptide) described herein. A complement-mediated disorder may be any disorder in which complement activation is known or suspected of being a contributing and/or at least partially causative factor in at least some subjects suffering from the disorder. In some embodiments the disorder is characterized by aberrantly elevated C3 activation. In some embodiments the disorder is characterized by aberrantly reduced level or activity of one or more complement regulatory proteins, e.g., due to a mutation in a gene encoding such protein or a mutation in a gene that encodes a protein that plays a role in the expression, localization, or activity of such protein or due to autoantibodies to such protein. In some embodiments the disorder is characterized by the presence of autoantibodies and/or immune complexes in the body, which may activate complement via, e.g., the classical pathway. Autoantibodies may, for example, bind to self antigens, e.g., on cells or tissues in the body, e.g., antigens in or on blood cells, blood vessels, skin, nerves, muscle, connective tissue, heart, kidney, or thyroid. In some embodiments the disorder is characterized by aberrantly increased activity of a C3 and/or C5 convertase, e.g., due to an autoantibody that stabilizes such convertase. which may lead to excessive C3 and/or C5 activation.

Certain disorders that may be treated using C3 and/or C3b binding polypeptides (or a protein comprising a C3 and/or C3b binding polypeptide) described herein are discussed below. It should be understood that such disorder can be treated using a C3 and/or C3b binding polypeptide that is fused or conjugated to a heterologous polypeptide, e.g., a VEGF inhibitor and/or a half-life extending polypeptide, or using a C3 and/or C3b binding polypeptide that is not fused or conjugated to a heterologous polypeptide. For convenience, disorders may be grouped by reference to a tissue, organ, or system that is often particularly affected in subjects suffering from the disorder. It will be appreciated that many disorders may affect multiple different tissues, organs, and/or body systems, and such classification(s) are not intended to be limiting.

### Blood-Related and Vessel-Related Disorders

In some embodiments, a C3 and/or C3b binding polypeptide (or a protein comprising a C3 and/or C3b binding polypeptide) is administered to a subject suffering from, or at risk of, a complement-mediated blood-related disorder, such as paroxysmal nocturnal hemoglobinuria (PNH), atypical hemolytic uremic syndrome (aHUS), autoimmune hemolytic anemia, chronic cold agglutinin disease, HELLP syndrome, and/or warm autoimmune hemolytic anemia. In some embodiments, a C3 and/or C3b binding polypeptide (or a protein comprising a C3 and/or C3b binding polypeptide) is administered to a subject suffering from, or at risk of, a complement-mediated disorder that affects the circulatory system. For example, in some embodiments, the disorder is thrombotic microangiopathy (TMA) or a vasculitis (e.g., IgA vasculitis) or other disorder associated with vessel inflammation, e.g., blood vessel and/or lymph vessel inflammation. In some embodiments, a vasculitis is polyarteritis nodosa, hypocomplementemic urticarial vasculitis, pulmonary vasculitis, Wegener's granulomatosis, giant cell arteritis, Churg-Strauss syndrome, microscopic polyangiitis, pauci-immune vasculitis, Henoch-Schonlein purpura, Takayasu's arteritis, Kawasaki disease, or Behcet's disease. In some embodiments, a disorder is TMA secondary to atypical hemolytic uremic syndrome. In some embodiments, a subject is positive for antineutrophil cytoplasmic antibody (ANCA).

### Eye Disorders

In some embodiments, a C3 and/or C3b binding polypeptide (or a protein comprising a C3 and/or C3b binding polypeptide) is administered to a subject for treatment of a complement-mediated eye disorder, such as macular degeneration (e.g., age-related macular degeneration (AMD) or Stargardt macular dystrophy), diabetic retinopathy, glaucoma, or uveitis (e.g., posterior uveitis or anterior uveitis). In some embodiments, a subject suffers from or is at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a C3 and/or C3b binding polypeptide (or a protein comprising a C3 and/or C3b binding polypeptide), as compared with control (e.g., patients given a sham administration). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and wet AMD. In some embodiments an eye has GA but not neovascular AMD.

In some embodiments an eye has wet AMD but not GA. A significant proportion of patients with wet AMD who are treated with anti-VEGF agents (the standard of care) develop GA within a period of 2-5 years after starting therapy. Furthermore, visual function outcome in wet AMD patients declines over time despite anti-VEGF treatment. Without wishing to be bound by any theory, treatment of wet AMD patients with proteins described herein comprising a C3 and/or C3b binding polypeptide and a VEGF inhibitor may provide a superior outcome relative to treatment with a VEGF inhibitor alone. Treatment with a protein comprising a C3 and/or C3b binding polypeptide and a VEGF inhibitor may delay or prevent emergence of GA relative to treatment with a VEGF inhibitor in the absence of a C3 inhibiting therapy.

In some embodiments a subject has a genotype associated with increased risk of AMD or increased risk of rapid progression of AMD. For example, the subject may have a polymorphism in a gene encoding a complement protein or complement regulatory protein, e.g., CFH, C3, factor B, wherein the polymorphism is associated with an increased risk of AMD.

In some embodiments, a subject has an eye disorder characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, the eye disorder is Behcet's disease. In some embodiments, the eye disorder is an eye disorder characterized by optic nerve damage (e.g., optic nerve degeneration), such as glaucoma. Additional eye disorders include, e.g., retinitis pigmentosa, macular edema, Vogt-Koyangi-Harada syndrome, birdshot retino-chorioditis, sympathetic ophthalmia, ocular dicatricial pemphigoid, ocular pemphigus, nonartertic ischemic optic neuropathy, post-operative inflammation, and retinal vein occlusion.

In some embodiments, a C3 and/or C3b binding polypeptide (or a protein comprising a C3 and/or C3b binding polypeptide) is introduced into the eye for treatment of an eye disorder such as macular degeneration (e.g., age-related macular degeneration (AMD) or Stargardt macular dystrophy), diabetic retinopathy, glaucoma, or uveitis. For example, a C3 and/or C3b binding polypeptide (or protein comprising a C3 and/or C3b binding polypeptide), may be introduced into the vitreous cavity (e.g., by intravitreal injection) for treatment of a subject suffering from or at risk of such an eye disorder, e.g., AMD. In some embodiments intravitreal administration is performed at intervals of at least 2 months. In some embodiments intravitreal administration is performed at intervals of at between 2 and 4 months, e.g., every 3 months. In some embodiments intravitreal administration is performed at intervals of at between 4 and 6 months. In some embodiments a protein comprising a C3 and/or C3b binding polypeptide, a VEGF inhibitor (e.g., ranibizumab), and one or more half-life extending polypeptides (e.g., having a structure as depicted in FIG. 11, 12, 13, or 14) may have a half-life in vitreous of at least about twice that of ranibizumab alone following intravitreal administration to a subject (e.g., a non-human primate or human subject). In some embodiments a protein comprising a C3 and/or C3b binding polypeptide, a VEGF inhibitor (e.g., ranibizumab), and one or more half-life extending polypeptides (e.g., having a structure as depicted in FIG. 11, 12, 13, or 14), may be administered by intravitreal administration at intervals of 2 months or more. In certain embodiments the dosing interval is about 3 months. In certain embodiments the dosing interval is about 4 months.

In some embodiments a C3 and/or C3b binding polypeptide (or protein comprising a C3 and/or C3b binding polypeptide), may be administered into the suprachoroidal space, e.g., by suprachoroidal injection to treat an eye disorder, e.g., any of the foregoing eye disorders. In some embodiments a C3 and/or C3b binding polypeptide (or protein comprising a C3 and/or C3b binding polypeptide) may be administered subretinally, e.g., by subretinal injection.

### Nervous System Disorders

In some embodiments, a C3 and/or C3b binding polypeptide (or protein comprising a C3 and/or C3b binding polypeptide) is used to treat a subject suffering from or at risk of a complement-mediated disorder that affects the nervous system, e.g., the central nervous system (CNS) and/or peripheral nervous system (PNS). Examples of such disorders include, e.g., a neurodegenerative disorder such as multiple sclerosis, other demyelinating diseases (e.g., neuromyelits optica or chronic inflammatory demyelinating polyneuropathy (CIDP)), amyotrophic lateral sclerosis, chronic pain, fibromyalgia, stroke, intracerebral hemorrhage, allergic neuritis, diabetic neuropathy, Huntington's disease, schizophrenia, Alzheimer's disease, Parkinson's disease, progressive supranuclear palsy, Lewy body dementia (i.e., dementia with Lewy bodies or Parkinson's disease dementia), frontotemporal dementia, progressive supranuclear palsy, corticobasal syndrome, Pick's disease, mild cognitive impairment, traumatic brain injury, traumatic spinal cord injury, multisystem atrophy, chronic traumatic encephalopathy, Creutzfeldt-Jakob disease, Guillain Barre Syndrome, glioblastoma, and leptomeningeal metastasis. In some embodiments, a subject suffers from neuropathic pain, e.g., arising from lesions that involve the somatosensory pathways with damage to small fibres in peripheral nerves and/or to the spino-thalamocortical system in the CNS.

### Kidney Disorders

In some embodiments, a C3 and/or C3b binding polypeptide (or protein comprising a C3 and/or C3b binding polypeptide) is used to treat a subject suffering from, or at risk of, a complement-mediated kidney disorder. Such disorders include, e.g., nephritis, e.g., glomerulonephritis, e.g., membranoproliferative glomerulonephritis (MPGN) (e.g., MPGN type I, MPGN type II, or MPGN type III), e.g., immune complex membranoproliferative glomerulonephritis (IC-MPGN). In some embodiments the disorder is IgA nephropathy (IgAN), primary membranous nephropathy, or diabetic nephropathy. In some embodiments, the disorder is polycystic kidney disease (PKD). In some embodiments, the disorder is C3 glomerulopathy. In some embodiments the disorder is characterized by glomerular deposits containing one or more complement activation products, e.g., C3b, in the kidney. In some embodiments treatment as described herein reduces the level of such deposits. In some embodiments a subject suffering from a complement-mediated kidney disorder suffers from proteinuria (an abnormally high level of protein in the urine) and/or an abnormally low glomerular filtration rate (GFR). In some embodiments treatment as described herein results in decreased proteinuria and/or an increased or stabilized GFR.

### Respiratory Disorders

In some embodiments, a C3 and/or C3b binding polypeptide (or protein comprising a C3 and/or C3b binding polypeptide) is used to treat a subject suffering from or at risk of a complement-mediated disorder respiratory disorder. In some embodiments, a subject is suffering from or at risk of acute respiratory distress syndrome. In some embodiments, a respiratory disease is, e.g., asthma (e.g., allergic asthma), emphysema, chronic inflammation, chronic obstructive pulmonary disease (COPD), pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, pulmonary embolisms and infarcts, dyspnea, hemoptysis, bronchoconstriction, or bronchiolitis obliterans.

### Musculoskeletal Disorders

In some embodiments, a C3 and/or C3b binding polypeptide (or protein comprising a C3 and/or C3b binding polypeptide) is used to treat a subject suffering from, or at risk of, a complement-mediated disorder that affects the musculoskeletal system. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, a complement-mediated musculoskeletal disorder is myasthenia gravis.

### Transplantation

In some embodiments, a C3 and/or C3b binding polypeptide (or protein comprising a C3 and/or C3b binding polypeptide) is used to protect a graft from complement-mediated damage. A graft can be contacted with a C3 and/or C3b binding polypeptide (or protein comprising a C3 and/or C3b binding polypeptide) prior to, during, and/or after being transplanted, in various embodiments of the disclosure. In another embodiment, a C3 and/or C3b binding polypeptide (or protein comprising a C3 and/or C3b binding polypeptide) is administered to a donor prior to removal of the graft. In some embodiments, a C3 and/or C3b binding polypeptide (or protein comprising a C3 and/or C3b binding polypeptide) is administered to a recipient during and/or after the introduction of the graft. In some embodiments, a C3 and/or C3b binding polypeptide (or protein comprising a C3 and/or C3b binding polypeptide) is administered to a recipient prior to the introduction of the graft. In some embodiments, a subject receives a C3 and/or C3b binding polypeptide (or protein comprising a C3 and/or C3b binding polypeptide) after receiving the graft.

In some embodiments, a graft is or comprises a solid organ such as a kidney, liver, lung, pancreas, or heart. In some embodiments, a graft is or comprises bone, cartilage, fascia, tendon, ligament, cornea, sclera, pericardium, skin, heart valve, blood vessel, amniotic membrane, or dura mater. In some embodiments, a graft comprises multiple organs such as a heart-lung or pancreas-kidney graft. In some embodiments, a graft comprises less than a complete organ or tissue. For example, a graft may contain a portion of an organ or tissue, e.g., a liver lobe, section of blood vessel, skin flap, or heart valve. In some embodiments, a graft comprises a preparation comprising isolated cells or tissue fragments that have been isolated from their tissue of origin but retain at least some tissue architecture, e.g., pancreatic islets. In some embodiments, a preparation comprises isolated cells that are not attached to each other via connective tissue, e.g., hematopoietic stem cells or progenitor cells derived from peripheral and/or cord blood, or whole blood or any cell-containing blood product such as red blood cells (RBCs) or platelets.

In some embodiments, a graft is a xenograft (i.e., the donor and recipient are of different species), an autograft (i.e., a graft from one part of the body to another part of the body in the same individual), an isograft (i.e., the donor and recipient are genetically identical), or an allograft (i.e., the donor and recipient are genetically non-identical members of the same species).

### Ischemia/Reperfusion Injury

Ischemia-reperfusion (I/R) injury is an important cause of tissue damage following trauma and in other conditions associated with temporary disruption of blood flow such as myocardial infarction, stroke, severe infection, vascular disease, aneurysm repair, cardiopulmonary bypass, and transplantation. In the setting of trauma, systemic hypoxemia, hypotension, and local interruption of the blood supply resulting from contusions, compartment syndrome, and vascular injuries cause ischemia that damages metabolically active tissues. Restoration of the blood supply triggers an intense systemic inflammatory reaction. After reperfusion, all three major complement pathways are activated and, acting cooperatively or independently, are involved in I/R related adverse events affecting numerous organ systems.

In some embodiments a C3 and/or C3b binding polypeptide (or protein comprising a C3 and/or C3b binding polypeptide) is administered to a subject who has recently (e.g., within the preceding 2, 4, 8, 12, 24, or 48 hours) experienced trauma, e.g., trauma that puts the subject at risk of I/R injury, e.g., due to systemic hypoxemia, hypotension, and/or local interruption of the blood supply. In some embodiments, a C3 and/or C3b binding polypeptide (or protein comprising a C3 and/or C3b binding polypeptide) may be administered intravascularly, optionally into a blood vessel that supplies an injured body part or directly to the body part. In some embodiments, the subject suffers from spinal cord injury, traumatic brain injury, burn, and/or hemorrhagic shock.

In some embodiments, a C3 and/or C3b binding polypeptide (or protein comprising a C3 and/or C3b binding polypeptide) is administered to a subject prior to, during, or after a surgical procedure, e.g., a surgical procedure that is expected to temporarily disrupt blood flow to a tissue, organ, or portion of the body. Examples of such procedures include cardiopulmonary bypass, angioplasty, carotid endarterectomy, heart valve repair/replacement, aneurysm repair, or other vascular surgeries. A C3 and/or C3b binding polypeptide (or protein comprising a C3 and/or C3b binding polypeptide) may be administered prior to, after, and/or during an overlapping time period with the surgical procedure.

In some embodiments, a C3 and/or C3b binding polypeptide (or protein comprising a C3 and/or C3b binding polypeptide) is administered to a subject who has suffered an MI, thromboembolic stroke, deep vein thrombosis, or pulmonary embolism. A C3 and/or C3b binding polypeptide (or protein comprising a C3 and/or C3b binding polypeptide) may be administered in combination with a thrombolytic agent such as tissue plasminogen activator (tPA) (e.g., alteplase (Activase), reteplase (Retavase), tenecteplase (TNKase)), anistreplase (Eminase), streptokinase (Kabikinase, Streptase), or urokinase (Abbokinase). In some embodiments, a C3 and/or C3b binding polypeptide (or protein comprising a C3 and/or C3b binding polypeptide) may be administered prior to, after, and/or during an overlapping time period with the thrombolytic agent.

### Other Disorders

In some embodiments, a C3 and/or C3b binding polypeptide (or protein comprising a C3 and/or C3b binding polypeptide) is used to treat a subject suffering from, or at risk of, a complement-mediated disorder that affects the integumentary system. Examples of such disorders include, e.g., atopic dermatitis, psoriasis, pemphigoid, pemphigus, systemic lupus erythematosus, dermatomyositis, scleroderma, sclerodermatomyositis, Sjögren syndrome, and chronic urticaria.

In some embodiments, a C3 and/or C3b binding polypeptide (or protein comprising a C3 and/or C3b binding polypeptide) is used to treat a subject suffering from, or at risk of, a complement-mediated disorder that affects the gastrointestinal system, e.g., inflammatory bowel disease, e.g., Crohn's disease or ulcerative colitis.

In some embodiments, a C3 and/or C3b binding polypeptide (or protein comprising a C3 and/or C3b binding polypeptide) is used to treat a subject suffering from, or at risk of, a complement-mediated inflammatory disorder, such as rhinosinusitis or myocarditis.

In some embodiments, a C3 and/or C3b binding polypeptide (or protein comprising a C3 and/or C3b binding polypeptide) is used to treat a subject suffering from, or at risk of, thyroiditis (e.g., Hashimoto's thyroiditis, Graves' disease, post-partum thyroiditis), pancreatitis, panniculitis, or MYH9-related disorders.

In some embodiments a C3 and/or C3b binding polypeptide (or protein comprising a C3 and/or C3b binding polypeptide) is used to treat a subject suffering from or at risk of a disorder affecting the liver. In some embodiments, the disorder is a fatty liver disorder. In some embodiments, a fatty liver disorder is non-alcoholic fatty liver disease (NAFL) (e.g., non-alcoholic steatohepatitis (NASH) or non-alcoholic fatty liver disease (NAFLD)) or alcoholic liver disease (e.g., alcoholic fatty liver disease (AFLD) or alcoholic steatohepatitis (ASH). In some embodiments the disorder is hepatitis (e.g., hepatitis C).

In some embodiments, a C3 and/or C3b binding polypeptide (or protein comprising a C3 and/or C3b binding polypeptide) is used to treat interleukin-2 induced toxicity during IL-2 therapy, myocardial infarction, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, liver fibrosis, fibrogenic dust diseases, nasal polyposis, parasitic diseases, Goodpasture's Syndrome, immune complex-associated inflammation, antiphospholipid syndrome, cancer, periodontitis, gingivitis, or obesity.

In some embodiments, a complement-mediated condition, disorder or disease that may be treated using a C3 and/or C3b binding polypeptide (or protein comprising a C3 and/or C3b binding polypeptide) is complement activation secondary to administration of another therapeutic or diagnostic agent. For example, in some embodiments, a complement-mediated condition, disorder or disease is complement activation secondary to gene therapy (e.g., gene therapy with a viral vector such as an adeno-associated virus (AAV), adenovirus, or lentivirus vector) or complement activation secondary to cell therapy). In some embodiments, a subject suffers from TMA secondary to hematopoietic stem cell transplant (HSCT-TMA). In some embodiments, a subject suffers from drug-induced TMA. In some embodiments, administration of a C3 and/or C3b binding polypeptide described herein prior to and/or following administration of another therapeutic agent may increase efficacy and/or safety of said therapeutic agent.

In some embodiments, a chronic complement-mediated disorder is characterized by the presence of autoantibodies and/or immune complexes in the body, which may activate complement via, e.g., the classical pathway. Autoantibodies may, for example, bind to self antigens, e.g., on cells or tissues in the body. In some embodiments, autoantibodies bind to antigens in blood vessels, skin, nerves, muscle, connective tissue, heart, kidney, thyroid, etc. In some embodiments, a subject has neuromyelitis optica and produces an autoantibody (e.g., an IgG autoantibody) to aquaporin 4. In some embodiments, a subject has pemphigoid and produces an autoantibody (e.g., an IgG or IgE autoantibody) to a structural component of the hemidesmosome (e.g., transmembrane collagen XVII (BP180 or BPAG2) and/or plakin family protein BP230 (BPAG1). In some embodiments, a chronic complement-mediated disorder is not characterized by autoantibodies and/or immune complexes.

### VII. COMBINATION THERAPY

In some aspects, methods of the present disclosure involve administering a C3 and/or C3b binding polypeptide (or protein comprising a C3 and/or C3b binding polypeptide), alone or in combination with one or more additional complement inhibitors. In some embodiments, a C3 and/or C3b binding polypeptide (or protein comprising a C3 and/or C3b binding polypeptide) is administered to a subject already receiving therapy with another complement inhibitor; in some embodiments, another complement inhibitor is administered to a subject receiving a C3 and/or C3b binding polypeptide (or protein comprising a C3 and/or C3b binding polypeptide). In some embodiments, both a C3 and/or C3b binding polypeptide (or protein comprising a C3 and/or C3b binding polypeptide) and another complement inhibitor are administered to the subject.

In some embodiments administration of a C3 and/or C3b binding polypeptide (or protein comprising a C3 and/or C3b binding polypeptide) may allow for administering a reduced dosing regimen of (e.g., involving a smaller amount in an individual dose, reduced frequency of dosing, reduced number of doses, and/or reduced overall exposure to) a second complement inhibitor, as compared to administration of a second complement inhibitor as single therapy. In some embodiments administration of a second complement inhibitor may allow for administering a reduced dosing regimen of (e.g., involving a smaller amount in an individual dose, reduced frequency of dosing, reduced number of doses, and/or reduced overall exposure to) a C3 and/or C3b binding polypeptide (or protein comprising a C3 and/or C3b binding polypeptide), as compared to administration of a second complement inhibitor as single therapy.

In some aspects, administration of a C3 and/or C3b binding polypeptide (or protein comprising a C3 and/or C3b binding polypeptide) in combination with a second complement inhibitor can reduce the amount of C3 or C3 activation in the subject's blood or in a relevant region of the body or on cells where inhibition of complement activity is desired sufficiently such that a reduced dosing regimen of a C3 and/or C3b binding polypeptide (or protein comprising a C3 and/or C3b binding polypeptide) and/or the second complement inhibitor is required to achieve a desired degree of complement inhibition.

In some embodiments such a reduced dose can be administered in a smaller volume, or using a lower concentration, or using a longer dosing interval, or any combination of the foregoing, as compared to administration of a C3 and/or C3b binding polypeptide (or protein comprising a C3 and/or C3b binding polypeptide) or a second complement inhibitor as single therapy.

Any complement inhibitor, e.g., a complement inhibitor known in the art, can be administered in combination with a C3 and/or C3b binding polypeptide (or protein comprising a C3 and/or C3b binding polypeptide) described herein. In some embodiments, a complement inhibitor is compstatin or a compstatin analog.

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 53), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus. The term “compstatin” is used herein consistently with such usage. Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345). Additional compstatin analogs are described in, e.g., WO 2012/155107, WO 2014/078731, and WO2019/166411.

In some embodiments, a complement inhibitor is an antibody, e.g., an anti-C3 and/or anti-C5 antibody, or a fragment thereof.

In some embodiments, an antibody fragment may be used to inhibit C3 or C5 activation. The fragmented anti-C3 or anti-C5 antibody may be a Fab, Fab′, Fab′(2), Fv, or single chain Fv. In some embodiments, the anti-C3 or anti-C5 antibody is monoclonal. In some embodiments, the anti-C3 or anti-C5 antibody is polyclonal. In some embodiments, the anti-C3 or anti-C5 antibody is de-immunized. In some embodiments the anti-C3 or anti-C5 antibody is a fully human monoclonal antibody. In some embodiments, the anti-C5 antibody is eculizumab. In some embodiments, a complement inhibitor is an antibody, e.g., an anti-C3 and/or anti-C5 antibody, or a fragment thereof.

In some embodiments, a complement inhibitor is a nucleic acid agent, e.g., a microRNA or an siRNA.

MicroRNAs (miRNAs) are a highly conserved class of small RNA molecules that are transcribed from DNA in the genomes of plants and animals, but are not translated into protein. Naturally occurring miRNAs are first transcribed as long hairpin-containing primary transcripts (pri-miRNAs). The primary transcript is cleaved by Drosha ribonuclease III enzyme to produce an approximately 70 nt stem-loop precursor miRNA (pre-miRNA), which includes an “antisense strand” or “guide strand” (that includes a region that is substantially complementary to a target sequence) and a “sense strand” or “passenger strand” (that includes a region that is substantially complementary to a region of the antisense strand). The pre-miRNA is then actively exported to the cytoplasm where it is cleaved by Dicer ribonuclease to form the mature miRNA. Processed microRNAs are incorporated into the RNA-induced silencing complex (RISC) to form mature gene-silencing complexes, which induce target mRNA degradation and/or translation repression. The number of miRNA sequences identified to date is large and growing, illustrative examples of which can be found, for example, in: “miRBase: microRIVA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S. NAR, 2004, 32, Database Issue, D109-D111.

In some embodiments, miRNAs can be synthesized and locally or systemically administered to a subject, e.g., for therapeutic purposes. miRNAs can be designed and/or synthesized as mature molecules or precursors (e.g., pri- or pre-miRNAs). In some embodiments, a pre-miRNA includes a guide strand and a passenger strand that are the same length (e.g., about 15, 16, 17, 18, 19, 20, 21, 22, 23, 24, or 25 nucleotides). In some embodiments, a pre-miRNA includes a guide strand and a passenger strand that are different lengths (e.g., one strand is about 19 nucleotides, and the other is about 21 nucleotides). In some embodiments, an miRNA can target the coding region, the 5′ untranslated region, and/or 3′ untranslated region, of endogenous mRNA. In some embodiments, an miRNA comprises a guide strand comprising a nucleotide sequence having sufficient sequence complementary with an endogenous mRNA of a subject to hybridize with and inhibit expression of the endogenous mRNA.

In some embodiments, an miRNA comprises a nucleic acid strand that is complementary to a target portion of a C3 transcript, e.g., C3 mRNA (e.g., complementary to a nucleotide sequence that is at least 90%, 91%, 92%, 93%, 94%, 95%, 96%, 97%, 98%, 99%, or 100% identical to a portion of human mRNA that encodes C3). The target portion may be 15-30 nucleotides long, e.g., 15, 16, 17, 18, 19, 20, 21, 22, 23, 24, 25, 26, 27, 28, 29, or 30 nucleotides long, although shorter and longer target portions are also contemplated. Human C3 is of particular interest herein. In some embodiments, the miRNA comprises a nucleic acid strand that comprises a region that is perfectly complementary to at least 6, 7, 8, 9, 10, 11, 12, 13 14, 15, 16, 17, 18, 19, 20, 21, 22, 23, 24, 25, 26, 27, 28, 29, or 30 consecutive nucleotides of human mRNA that encodes C3. The amino acid and nucleotide sequences of human C3 are known in the art and can be found in publicly available databases, for example, the National Center for Biotechnology Information (NCBI) Reference Sequence (RefSeq) database, where they are listed under RefSeq accession numbers NP_000055 (accession.version number NP_000055.2) and NM_000064 (accession.version number NM_000064.4), respectively (where “amino acid sequence” refers to the sequence of the C3 polypeptide and “nucleotide sequence” in this context refers to the C3 mRNA sequence as represented in genomic DNA, it being understood that the actual mRNA nucleotide sequence contains U rather than T). One of ordinary skill in the art will appreciate that the afore-mentioned sequences are for the complement C3 preproprotein, which includes a signal sequence that is cleaved off and is therefore not present in the mature protein. The human C3 gene has been assigned NCBI Gene ID: 718, and the genomic C3 sequence has RefSeq accession number NG_009557 (accession.version number NG_009557.1).

RNA interference (RNAi) is a process of sequence-specific post-transcriptional gene silencing by which, e.g., double stranded RNA (dsRNA) homologous to a target locus can specifically inactivate gene function (Hammond et al., Nature Genet. 2001; 2:110-119; Sharp, Genes Dev. 1999; 13:139-141). This dsRNA-induced gene silencing can be mediated by short double-stranded small interfering RNAs (siRNAs) generated from longer dsRNAs by ribonuclease III cleavage (Bernstein et al., Nature 2001; 409:363-366 and Elbashir et al., Genes Dev. 2001; 15:188-200). RNAi-mediated gene silencing is thought to occur via sequence-specific RNA degradation, where sequence specificity is determined by the interaction of an siRNA with its complementary sequence within a target RNA (see, e.g., Tuschl, Chem. Biochem. 2001; 2:239-245). RNAi can involve the use of, e.g., siRNAs (Elbashir, et al., Nature 2001; 411: 494-498) or short hairpin RNAs (shRNAs) bearing a fold back stem-loop structure (Paddison et al., Genes Dev. 2002; 16: 948-958; Sui et al., Proc. Natl. Acad. Sci. USA 2002; 99:5515-5520; Brummelkamp et al., Science 2002; 296:550-553; Paul et al., Nature Biotechnol. 2002; 20:505-508).

In some embodiments an siRNA targets C3 transcript, e.g., C3 mRNA (SEQ ID NO:45). In some embodiments, siRNAs are double stranded nucleic acid duplexes (of, e.g., 15, 16, 17, 18, 19, 20, 21, 22, 23, 24, 25, 26, or 27 base pairs) comprising annealed complementary single stranded nucleic acid molecules. In some embodiments, the siRNAs are short dsRNAs comprising annealed complementary single strand RNAs. In some embodiments, the siRNAs comprise an annealed RNA:DNA duplex, wherein the sense strand of the duplex is a DNA molecule and the antisense strand of the duplex is a RNA molecule.

In some embodiments, duplexed siRNAs comprise a 2 or 3 nucleotide 3′ overhang on each strand of the duplex. In some embodiments, siRNAs comprise 5′-phosphate and 3′-hydroxyl groups.

In some embodiments, an siRNA molecule includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Exemplary modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313.

In some embodiments, siRNA molecules comprise nucleosides that incorporate modified nucleobases and/or nucleobases covalently bound to modified sugars. Some examples of nucleosides that incorporate modified nucleobases include 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

Methods of preparing modified nucleobases are described in, e.g., U.S. Pat. Nos. 3,687,808; 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088.

In some embodiments, the 2′-OH of a ribose is replaced with a substituent including one of the following: —H, —F; —CF₃, —CN, —N₃, —NO, —NO₂, —OR′, —SR′, or —N(R′)₂, wherein each R′ is independently —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C2-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe. One or more such modifications may be incorporated at any position in various embodiments.

In some embodiments, an siRNA described herein can be introduced to a target cell as an annealed duplex siRNA. In some embodiments, an siRNA described herein is introduced to a target cell as single stranded sense and antisense nucleic acid sequences that, once within the target cell, anneal to form an siRNA duplex. Alternatively, the sense and antisense strands of the siRNA can be encoded by an expression vector (such as an expression vector described herein) that is introduced to the target cell. Upon expression within the target cell, the transcribed sense and antisense strands can anneal to reconstitute the siRNA.

An siRNA molecule described herein can be synthesized by standard methods known in the art, e.g., by use of an automated synthesizer. RNAs produced by such methodologies tend to be highly pure and to anneal efficiently to form siRNA duplexes. Following chemical synthesis, single stranded RNA molecules can be deprotected, annealed to form siRNAs, and purified (e.g., by gel electrophoresis or HPLC). Alternatively, standard procedures can be used for in vitro transcription of RNA from DNA templates, e.g., carrying one or more RNA polymerase promoter sequences (e.g., T7 or SP6 RNA polymerase promoter sequences). Protocols for preparation of siRNAs using T7 RNA polymerase are known in the art (see, e.g., Donze and Picard, Nucleic Acids Res. 2002; 30:e46; and Yu et al., Proc. Natl. Acad. Sci. USA 2002; 99:6047-6052). The sense and antisense transcripts can be synthesized in two independent reactions and annealed later, or they can be synthesized simultaneously in a single reaction.

An siRNA molecule can also be formed within a cell by transcription of RNA from an expression construct introduced into the cell (see, e.g., Yu et al., Proc. Natl. Acad. Sci. USA 2002; 99:6047-6052). An expression construct for in vivo production of siRNA molecules can include one or more siRNA encoding sequences operably linked to elements necessary for the proper transcription of the siRNA encoding sequence(s), including, e.g., promoter elements and transcription termination signals. Preferred promoters for use in such expression constructs include the polymerase-Ill HI-RNA promoter (see, e.g., Brummelkamp et al., Science 2002; 296:550-553) and the U6 polymerase-Ill promoter (see, e.g., Sui et al., Proc. Natl. Acad. Sci. USA 2002; Paul et al., Nature Biotechnol. 2002; 20:505-508; and Yu et al., Proc. Natl. Acad. Sci. USA 2002; 99:6047-6052). An siRNA expression construct can further comprise one or more vector sequences that facilitate the cloning of the expression construct. Standard vectors that can be used include, e.g., pSilencer 2.0-U6 vector (Ambion Inc., Austin, Tex.).

In some embodiments, an miRNA or siRNA described herein is delivered to a subject (e.g., to a cell of a subject, e.g., a liver cell of a subject) using an expression vector. Many forms of vectors can be used to deliver an miRNA or siRNA described herein. Non-limiting examples of expression vectors include viral vectors (e.g., vectors suitable for gene therapy), plasmid vectors, bacteriophage vectors, cosmids, phagemids, artificial chromosomes, and the like.

In some embodiments, a nucleotide sequence encoding an miRNA or siRNA described herein is integrated into a viral vector, e.g., a viral vector described herein.

In some embodiments, a vector comprises multiple nucleotide sequences, where each nucleotide sequence encodes a different miRNA or siRNA described herein. In some embodiments, a vector comprises multiple nucleotide sequences encoding at least 2 different miRNAs or siRNAs, wherein at least two of the nucleotide sequences are copies of the same miRNA or siRNA.

In some embodiments, in addition to one or more sequences encoding one or more miRNAs or siRNAs that inhibit C3 expression, a vector (e.g., a viral vector) comprises a nucleic acid sequence encoding a C3 and/or C3b binding polypeptide (or protein comprising a C3 and/or C3b binding polypeptide) described herein. In some embodiments a single expression vector may encode both a C3 and/or C3b binding polypeptide (or protein comprising a C3 and/or C3b binding polypeptide) and a miRNA, miRNA precursor, or siRNA targeted to C3. In some embodiments an expression vector may comprise a first promoter operably linked to a sequence encoding a C3 and/or C3b binding polypeptide (or protein comprising a C3 and/or C3b binding polypeptide) and a second promoter operably linked to a sequence encoding a miRNA or miRNA precursor. Other complement inhibitors that could be administered in combination with C3 and/or C3b binding polypeptides (or proteins comprising a C3 and/or C3b binding polypeptide) include, e.g., an anti-C3 or anti-C3b antibody (e.g., scFv or single domain antibody, e.g., a nanobody), an enzyme that degrades C3 or C3b (see, e.g., U.S. Pat. No. 6,676,943), or a mammalian complement regulatory protein (e.g., CR1, DAF, MCP, CFH, CFI, C1 inhibitor (C1-INH), a soluble form of complement receptor 1 (sCR1), TP10 or TP20 (Avant Therapeutics), or portion thereof. Additional polypeptide inhibitors include mini-factor H (see, e.g., U.S. Pub. No. 20150110766), Efb protein or complement inhibitor (SCIN) protein from Staphylococcus aureus, or a variant or derivative or mimetic thereof (see, e.g., U.S. Pub. No. 20140371133).

In some embodiments, a polypeptide is linked to a secretion signal sequence for secretion of the expressed polypeptide from a host cell.

Inhibitors of C1s may be of use in certain embodiments. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 46) or KTISKAKGQPREPQVYT (SEQ ID NO: 47) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### VIII. EXEMPLIFICATION

### Example 1: Production of Polypeptides with Improved C3 and/or C3b Binding

The present Example demonstrates engineering of therapeutic polypeptides that specifically bind to complement component 3 (C3) and/or C3b and exhibit inhibition of both the classical and alternative complement pathways. Nanofitins are variants of wild-type Sac7d protein from Sulfolobus acidocaldarius (NCBI: AAA80315) (or variants of other wild type Sac7d family members) that are engineered to bind to particular targets. In the present Example, Nanofitin polypeptides based on the reference amino acid sequence of Sac7d (SEQ ID NO:1) were engineered to include mutations that enhance binding to C3 and/or C3b.

Nanofitins were derived from Sac7d by reengineering the natural binding site. Randomization of the residues exposed to solvent allowed for the creation of libraries of Nanofitin variants from the wild-type Sac7d protein. Libraries were reengineered so that the polypeptides no longer recognize DNA, and instead bound specifically with C3 and/or C3b. The resulting Nanofitin polypeptides combine specific, high-affinity binding to targets with the original stability features of the parent protein (e.g., thermophilic and acidophilic).

A comprehensive characterization of 20 clones was performed to screen for the following characteristics: affinity for C3 of below 10 nM and inhibition of the complement cascade. Polypeptides having the best characteristics of neutralizing complement activation were chosen to be matured. Maturation included two sequential steps shown below.

STEP 1: Binding site Alanine scanning, with C3 binding measured by ELISA

STEP 2: Further maturation with key binding residues fixed, and secondary residues randomized followed by ribosome display for selection of candidates with a low K_{off} (dissociation measured by Bio-layer interferometry (BLI)).

The polypeptide having the amino acid sequence of SEQ ID NO: 27 was affinity matured to generate the polypeptides comprising the amino acid sequences of SEQ ID NOs: 33 and 34. Based on the predominant appearance of the W24Y and R42W mutations among the variants and the finding that changing Y24 and W42 to alanine abolished binding, these residues may be particularly important in conferring C3 and C3b binding and complement inhibiting activity to the variants.

### Example 2. Production of C3 and/or C3b Binding Polypeptides in CHO Cells

C3 and/or C3b binding polypeptides, dimers of C3 and/or C3b binding polypeptides, and fusion polypeptides were produced in Chinese Hamster Ovary (CHO) cells by expression with a His tag using the pCDNA 3.1(+) vector. Fab fusion proteins were generated in which the polypeptide having the amino acid sequence of SEQ ID NO: 27 was fused to polypeptide chains of a Fab antibody fragment comprising a heavy chain having the amino acid sequence of SEQ ID NO: 36 and a light chain having the amino acid sequence of SEQ ID NO: 35 (ranibizumab (Lucentis®, Genentech)). The Fab fusion proteins contained the polypeptide having the amino acid sequence of SEQ ID NO: 27 fused to the carboxyl terminus of SEQ ID NO:36 at position A (“Fab NF-A”), the CL of SEQ ID NO:35 at position C (“Fab NF-C”), or the VL of SEQ ID NO:35 at position D (“Fab NF-D”), as shown in FIG. 1. The polypeptide having the amino acid sequence of SEQ ID NO: 27 was also fused to the VH of SEQ ID NO:36 at position B, but had very low expression, and thus, was not further evaluated. The Fab fusion proteins included a flexible linker of 15 amino acids between the antibody chain and the Nanofitin and an IL2 signal sequence (IL2ss). An IL2ss was located at the N-terminus of each heavy and light chain (cleaved off prior to secretion).

### Example 3: ELISA-Based Assay of Complement Activation

An ELISA-based complement activation assay was used to determine inhibition of both the classical and alternative complement pathways for the polypeptides described in Example 1. A schematic of the role of C3 in both classical and alternative complement pathways and the downstream production of C5b-9 is shown in FIG. 2.

Antibodies labeled for C5b-9 neoantigen were used. The amount of neoantigen generated was proportional to the functional activity of complement pathways. Wells of the microtiter strips were coated with specific activators of the classical or alternative complement pathway. Serum was diluted in diluent containing specific blocker to ensure that only the respective pathway is activated. During incubation of diluted serum in the wells, complement was activated by the specific coating. C5b-9 was then detected with a specific alkaline phosphatase labelled antibody to the neoantigen expressed during MAC formation. A Nanofitin® control was included in all assays.

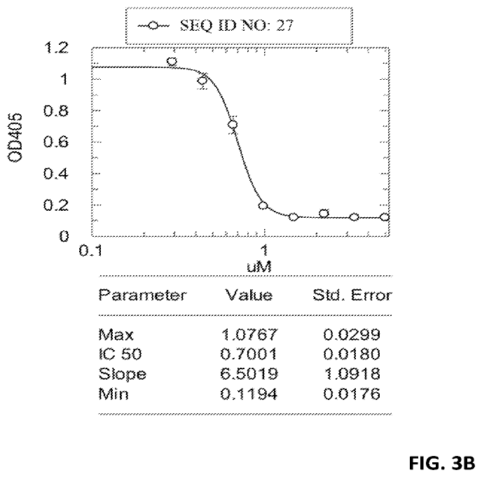

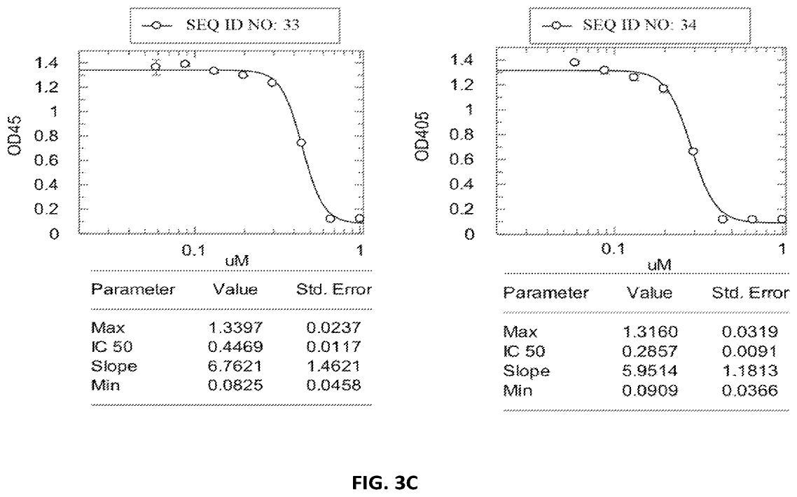

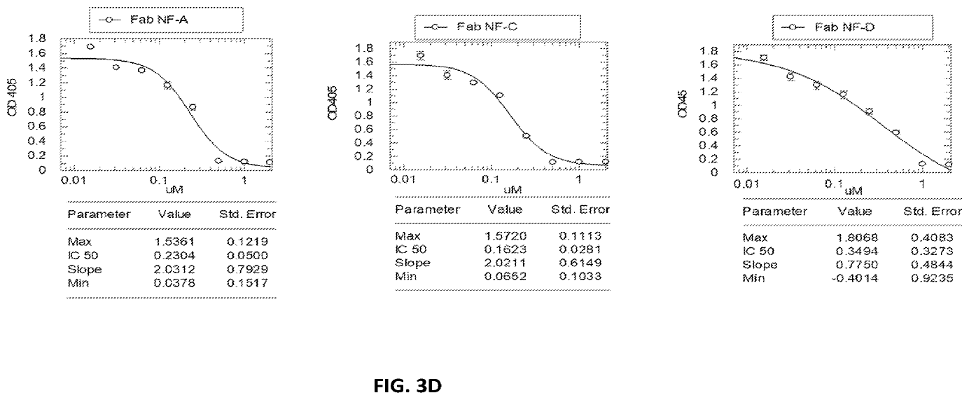

Complement activation correlates with the color intensity measured in absorbance (optical density), as shown in FIGS. 3A-3D. The IC50 (in nM) results for both classical and alternative complement pathways are shown in Table 20 below and indicate that the polypeptides described in Example 1 inhibit complement activation.

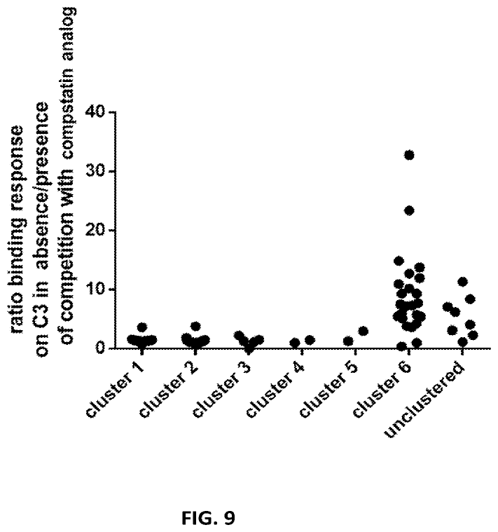

A competition ELISA was also performed to compare the binding of C3 and/or C3b binding polypeptides with an exemplary pegylated compstatin analog including a 10 kD PEG and having a structure as depicted in FIG. 8). All polypeptides plotted in FIG. 9 bound to C3b. A ratio of 1 in this competition ELISA meant that there was no competition between the polypeptide and the compstatin analog. A high ratio indicates that there was efficient competition. Competition was mostly observed in the family dubbed “cluster 6” in FIG. 9. Cluster 6 was the family of the polypeptide having the amino acid sequence of SEQ ID NO: 27, which is the parental clone used to generate the amino acid sequence of SEQ ID NO: 33 and SEQ ID NO: 34 by affinity maturation.

### Example 4: Inhibition of VEGF-Induced Endothelial Cell Proliferation by Fab Fusion Proteins

The effects of the Fab fusion proteins described in Example 1 on induction or inhibition of cellular proliferation was evaluated by using the classical MTT assay, as described previously (see Wang et al., Int J Ophthalmol. 2017; 10(8): 1212-1216). The reduction of tetrazolium salts is widely accepted as a reliable way to examine cell proliferation. The yellow tetrazolium MTT is reduced by metabolically active cells to generate reducing equivalents such as NADH and NADPH. The resulting intracellular purple formazan can be solubilized and quantified by spectrophotometry.

The HUVEC/TERT2 cell line has previously been established by ectopic expression of human telomerase reverse transcriptase (hTERT) in normal umbilical vein endothelial cells. HUVEC/TERT2 cells can be grown for more than 70 population doublings without showing signs of growth retardation and a stable growth rate (by comparison, the parental cell line reaches senescence at 33 doublings). Human hTERT immortalized endothelial cells (HUVEC/TERT2) were seeded into 96-well plates and treated with VEGF (15 ng/mL) together with the Fab fusion proteins described in Example 1 at increasing concentrations. Induction or inhibition of cellular proliferation was measured spectrophotometrically by reduction of MTT.

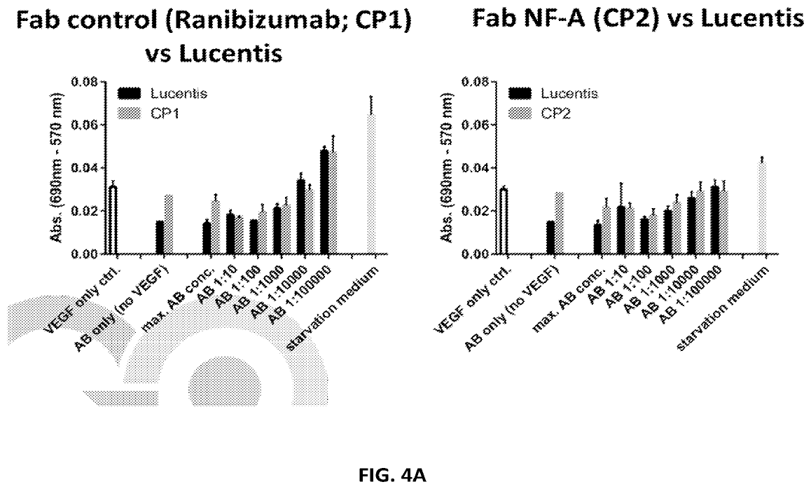

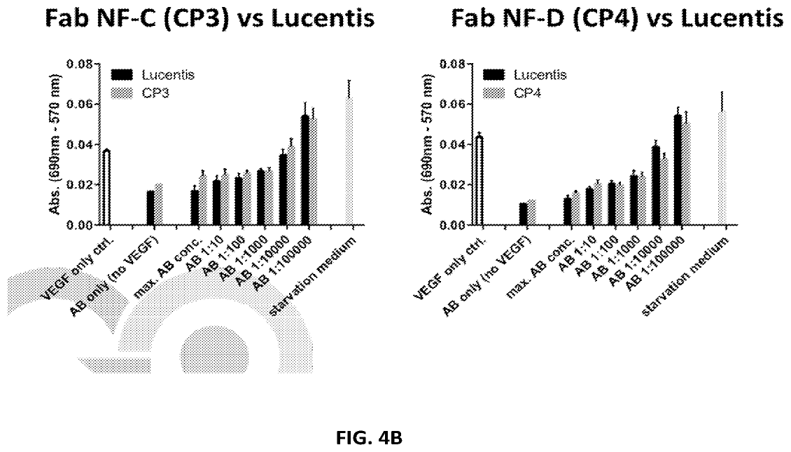

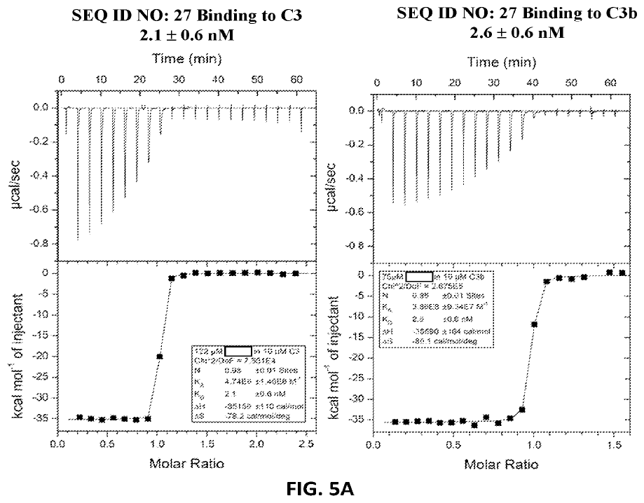

VEGF at a concentration of 15 ng/ml resulted in higher cell numbers compared to treatment with Lucentis® in absence of VEGF. Lucentis® (used as a positive control for inhibiting activity of VEGF) showed a clear and dose dependent effect. The test Fab fusion proteins showed a clear and dose dependent effect on VEGF similar to Lucentis®. In addition, both Fab NF-A and Lucentis® appeared to have a stimulatory effect on HUVEC/TERT2 cells in the absence of VEGF. Overall, each of the Fab fusion proteins (Fab NF-A, Fab NF-C, and Fab NF-D) inhibited VEGF-induced proliferation at a level similar to Lucentis®, as shown in FIGS. 4A-4B and Table 20 below.

### Example 5: Kinetic Assays of C3 and/or C3b Binding Polypeptides

C3 binding, C3b binding, and equilibrium dissociation constant (K_{D}) measurements were determined by Surface Plasmon Resonance (SPR) using the Sierra SPR-23 instrument. C3 and C3b proteins were obtained from Complement Technology, Inc. (Cat #A113c and A114). The polypeptides described in Example 1 were stored each at 1 mg/mL in PBS, pH 7.4.

Surface plasmon resonance (SPR) requires the attachment of the ligand to a surface and then flowing the analytes across this surface to measure the kinetic rate of the association (kₐ) and the dissociation (k_{d}) of the ligand with the protein. From this measurement, the K_{D} is modeled. Proteins were immobilized on a polystyrene or carboxymethylated dextran biosensor surface by pre-concentrating under various conditions and then amine-coupled to the surface. Upon attachment of the proteins to the surface, experiments with a known binder were performed to confirm that the attached protein retained this ability/activity and the relative coupling efficiencies compared under the various conditions. The polypeptides were evaluated using a serial dilution (typically a 6-point, 3-fold serial dilution assay performed in duplicate). The interaction parameters kₐ, k_{d}, and K_{D} were then determined.

More specifically, the SPR-32 instrument was first cleaned to ensure fluidics were free of any build up. Next, a new biosensor chip was docked and conditioned. Ligand (i.e., C3 or C3b) was immobilized to the CM5 biosensor chip surface at a level that resulted in an appropriate theoretically Rmax for the experiment, typically ˜100 RU. This was achieved by taking the ratio of the molecular weights of the ligand/analyte and multiplying by the desired Rmax. The experimentally extrapolated Rmax was then compared to the theoretical Rmax to obtain an estimate of the percent of surface bound protein that is active for binding. A pH-scouting test for C3 and C3b was performed at 1 M using 10 mM sodium acetate buffer. Sodium acetate pH 4.0 was observed to give best levels of protein immobilization.

C3 and C3b were placed onto Spots A and B across all 8 channels to test for the binding of the supplied analytes. Spot C was used as a reference spot. The conditioned biosensor chip was chemically activated with EDC/NHS. Ligands were then injected onto the activated surface to allow for immobilization. After immobilization, 1 M ethanolamine was injected to block any remaining reactive carboxyl groups in the carboxymethylated dextran surface.

In summary, ligands were immobilized under the following conditions: 25° C. on a Sierra Sensors HCA (CM5), Running Buffer of PBST (10 mM Phosphate Buffer pH 7.4, 150 mM NaCl, 0.05% Tween-20; pH 7.4), amine coupling Immobilization Buffer of 10 mM NaOAc pH 4.0, activation with EDC/NHS 6 minutes, immobilization for 1 minute, and blocking with 1 M ethanolamine.

The single cycle kinetic method was used to characterize the binding interactions of the proteins. For this method, analyte injections were injected in increasing concentration without regeneration and with shorter dissociation to calculate the association rates. The top concentration was injected at the end, but with longer dissociation time. The dissociation rate was calculated from the top concentration sensorgram, as dissociation rate is independent of the concentration. The signal was observed to create a stair-step pattern, where increasing amounts of analyte remained bound to the surface with each injection. The benefit from this approach is the increase in throughput and the avoidance of regeneration solutions that may be detrimental to the immobilized protein on the surface.

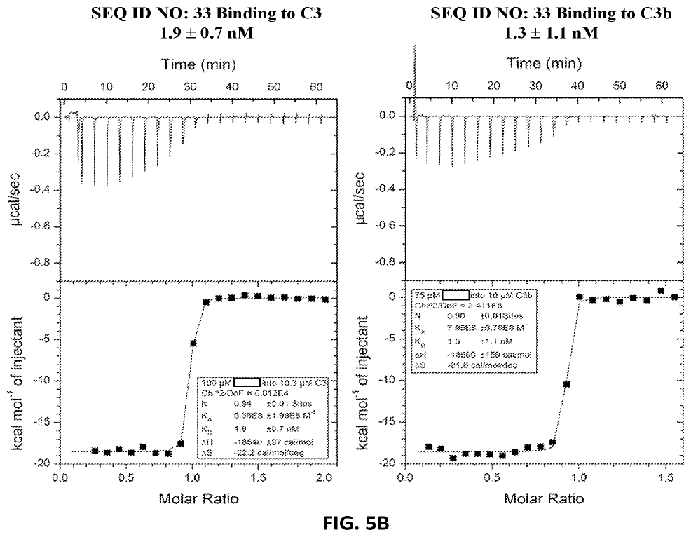

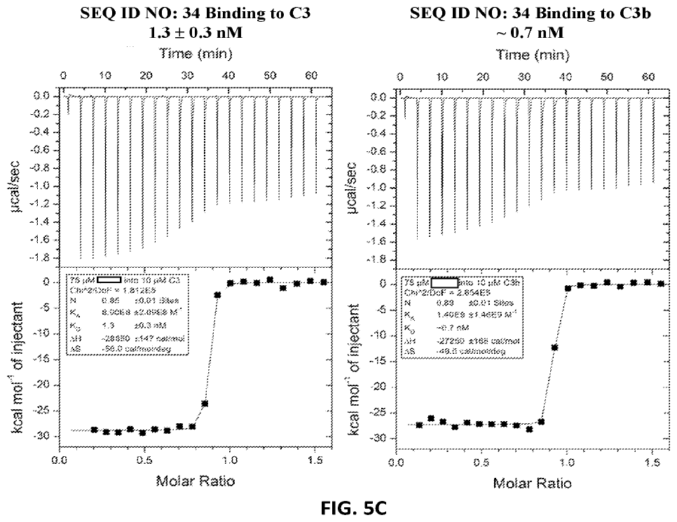

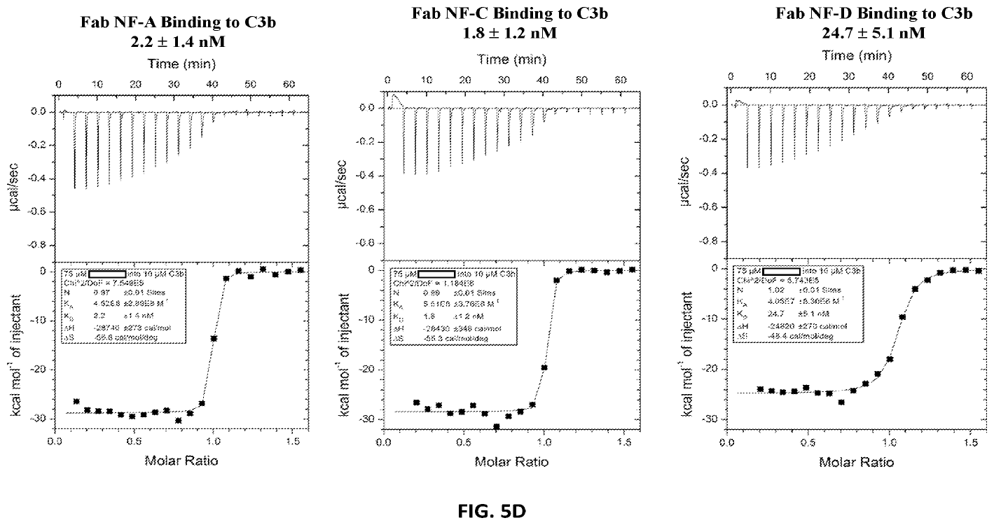

An 8-point, 3-fold serial dilution assay was performed in duplicate for the polypeptides described in Example 1 starting at either 20 nM, 50 nM, or 200 nM. The starting concentration was adjusted for each Nanofitin based on the K_{D} obtained by ITC, as shown in Table 19 and FIGS. 5A-5D.

TABLE 19

| | | | ΔG | ΔH | −TΔS |
|---|---|---|---|---|---|
| | | K_{D} | (kcal/ | (kcal/ | (kcal/ |
| titration | N | (nM) | mol) | mol) | mol) |
| SEQ ID NO: 27 into C3 | 0.98 | 2.1 | −11.8 | −35.2 | 23.4 |
| SEQ ID NO: 27 into C3b | 0.95 | 2.6 | −11.7 | −35.6 | 23.9 |
| SEQ ID NO: 33 into C3 | 0.94 | 1.9 | −11.9 | −18.5 | 6.6 |
| SEQ ID NO: 33 into C3b | 0.9 | 1.3 | −12.1 | −18.6 | 6.5 |
| SEQ ID NO: 34 into C3 | 0.85 | 1.3 | −12.1 | −28.8 | 16.7 |
| SEQ ID NO: 34 into C3b | 0.89 | 0.7 | −12.5 | −27.2 | 14.7 |
| Fab NF-A into C3b | 0.97 | 2.2 | −11.8 | −28.7 | 16.9 |
| Fab NF-C into C3b | 0.99 | 1.8 | −11.9 | −28.4 | 16.5 |
| Fab NF-D into C3b | 1.02 | 24.7 | −10.4 | −24.8 | 14.4 |

Sensorgrams were analyzed using Sierra Sensors Analyzer software version 3.1.33 with a double reference to determine the interaction parameters ka, kd, and K_{D}. The reference channel was first subtracted from the ligand channel as the first reference. The internal blank injections were set to closest blank after injection based on the method. The kd was calculated for the top concentration by fitting the off-rate part of the curve. The kd was then set to global constant for fitting rest of the curves. Rmax was fit locally for each individual curve since the number of available binding sites changed after each injection. Binding data was fit to a 1:1 Langmuir model.

The use of single cycle kinetics method yielded data that fit the 1:1 Langmuir model with low nanomolar affinities for the tested polypeptides against both C3 and C3b proteins. The control Nanofitin® did not bind to either of the proteins. This method resulted in a variable surface for each injection requiring the fitting of data with a local Rmax. Each polypeptide was run in duplicate over the same spot. Some duplicate titration showed lowered RU's when compared with the first run. This is due to less availability of free binding sites for the duplicate run. Despite the decreased RU values for the duplicate run, the data fit well with local Rmax analysis, adding confidence to the data.

A polypeptide having the amino acid sequence of SEQ ID NO: 27 bound to both proteins with similar affinities. Among the tested proteins comprising SEQ ID NO: 27 and an antigen binding fragment, NF Fab-D was observed to bind the weakest, with faster dissociation rate.

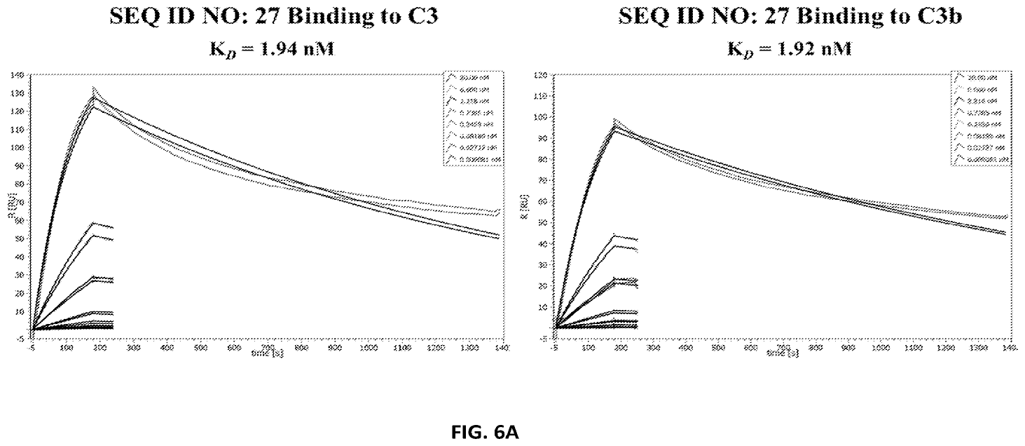

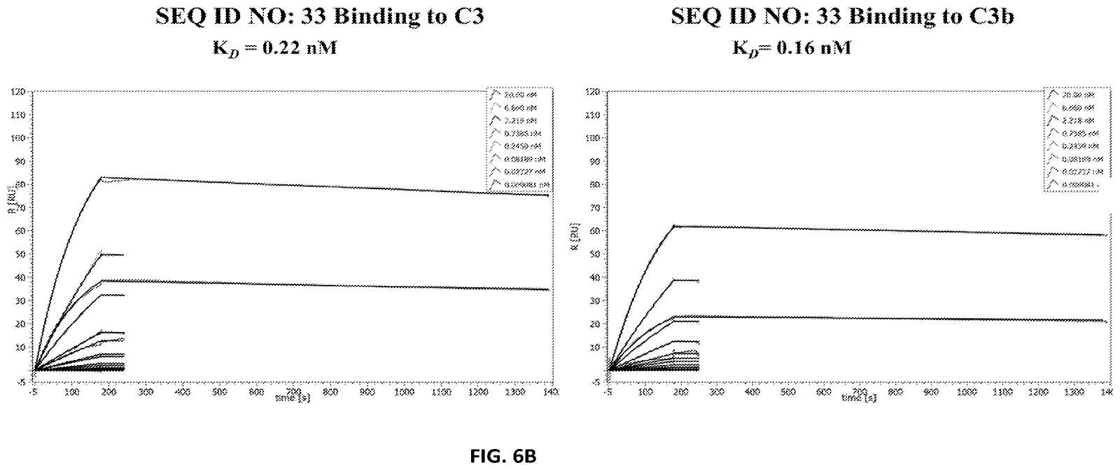

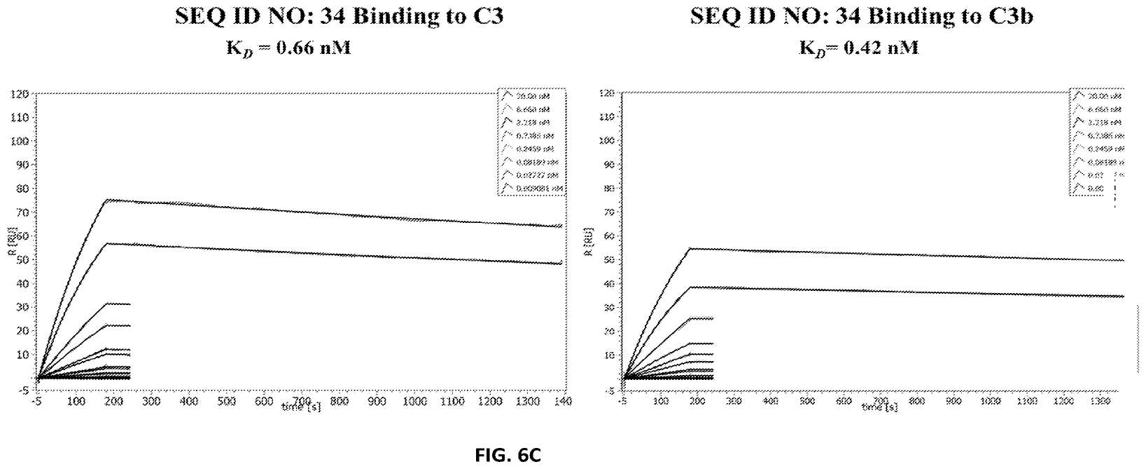

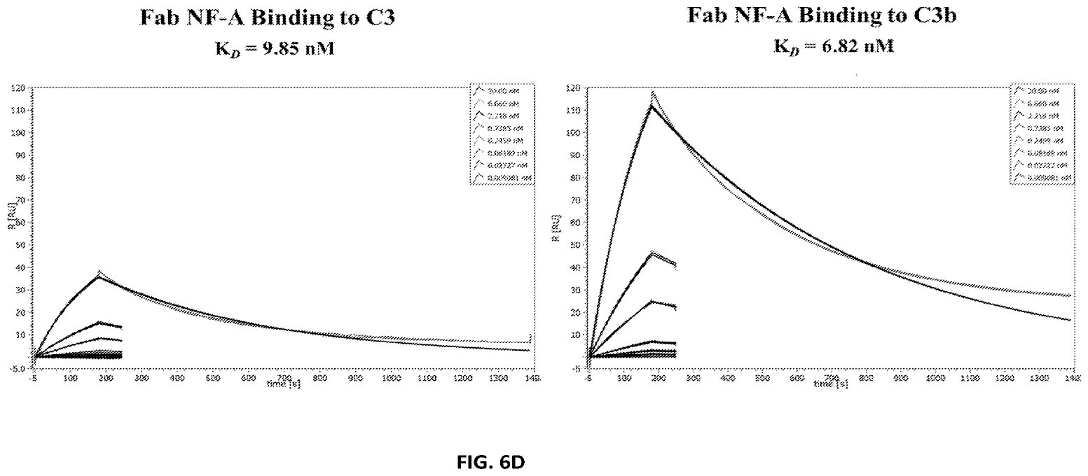

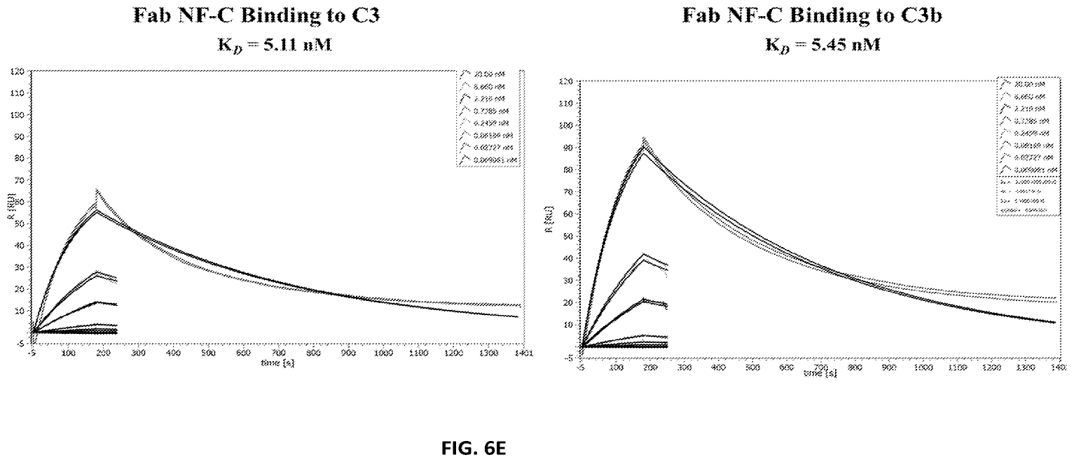

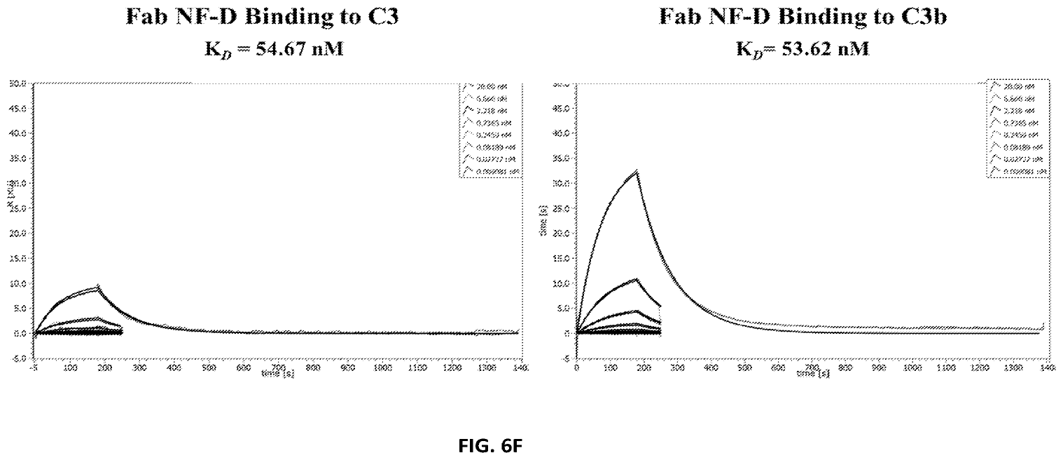

Sensograms for the polypeptides described in Example 1 showing binding to C3 and C3b and K_{D} are shown in FIGS. 6A-6F with results shown in Table 20.

Table 20 below shows IC50 values and the equilibrium dissociation constants (K_{D}) of the polypeptides described in Example 1. These results indicate that the polypeptides described herein have binding activity for C3 and C3b in the low nM range. All of the polypeptides were found to be active in inhibiting the classical and alternative complement pathways with IC50 values ranging from 60 nM to 1,759 nM. These results indicate that the polypeptides are biologically active in the nM range. Complement inhibiting activity of one of the affinity matured polypeptides was confirmed in a Wieslab® assay.

TABLE 20

| | | | | | ITC | | SPR | |
|---|---|---|---|---|---|---|---|---|
| | | | | VEGF Inhib. | K_{D} (nM) | K_{D} (nM) | K_{D} (nM) | K_{D} (nM) |
| | MW | IC50 (nM) | IC50 (nM) | Anti- | Binding | Binding | Binding | Binding |
| | (kDa) | Classical | Alternative | proliferation | to C3 | to C3b | to C3 | to C3b |
| SEQ ID | 8.8 | 743 | 700 | NA | 2.1 ± 0.6 | 2.6 ± 0.6 | 1.94 | 1.92 |
| NO: 27 | | | | | | | | |
| SEQ ID | 8.6 | 63 | 447 | NA | 1.9 ± 0.7 | 1.3 ± 1.1 | 0.22 | 0.16 |
| NO: 33 | | | | | | | | |
| SEQ ID | 8.6 | 79 | 286 | NA | 1.3 ± 0.3 | ~0.7 | 0.66 | 0.42 |
| NO: 34 | | | | | | | | |
| Fab NF-A | 56.8 | 232 | 230 | Similar to | NA | 2.2 ± 1.4 | 9.85 | 6.82 |
| | | | | Lucentis | | | | |
| Fab NF-C | 56.8 | 217 | 162 | Similar to | NA | 1.8 ± 1.2 | 5.11 | 5.45 |
| | | | | Lucentis | | | | |
| Fab NF-D | 56.8 | 1,759 | 349 | Similar to | NA | 24.7 ± 5.1 | 54.67 | 53.62 |
| | | | | Lucentis | | | | |
| Ranibizumab | 48 | NA | NA | Similar to | NA | NA | NA | NA |
| | | | | Lucentis | | | | |
| NF control | 8.8 | No | No | NA | No | No | No | No |
| | | activity | activity | | binding | binding | binding | binding |

NA = Not tested in this assay

### Example 6: Proteins with Complement Inhibiting Activity and VEGF Inhibiting Activity and Extended Half-Life

A Fab fusion protein (a modified Fab) having the structure depicted at the far left of FIG. 14 comprising the heavy and light chains of ranibizumab, with identical half-life extending polypeptides fused to the C-terminus of the heavy and light chains via a peptide linker, and one of the affinity matured C3 and/or C3b binding polypeptides described in Example 1 fused to the C-terminus of each half-life extending polypeptide via a peptide linker was produced by expression in CHO cells. The two C3 and/or C3b binding polypeptides had the same sequence. The ability of the protein (referred to herein as RNF-LA2) to inhibit complement activation was tested using Wieslab® assays in comparison with the C3 inhibitor pegcetacoplan. The ability of RNF-LA2 to inhibit the classical and alternative complement pathways was at least equal to that of pegcetacoplan.

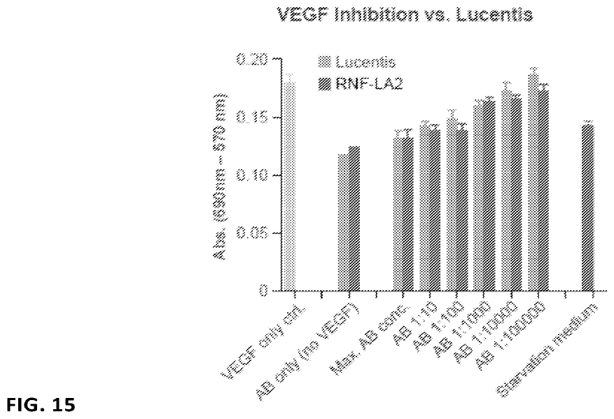

The ability of RNF-LA2 to inhibit VEGF was tested using the assay described in Example 4. As shown in FIG. 15, RNF-LA2 demonstrated comparable VEGF inhibiting activity to that of Lucentis®.

### Example 7: Intravitreal Administration of Protein with Complement Inhibiting Activity and VEGF Inhibiting Activity and Extended Half-Life

To assess pharmacokinetic properties of the Fab fusion protein described in Example 6 (RNF-LA2), cynomolgus monkeys were dosed by intravitreal injection with the same amount of either RNF-LA2 or Lucentis®. Levels of RNF-LA2 and Lucentis® in vitreous humor were measured at various timepoints following injection. The half-life of RNF-LA2 was about twice that of Lucentis®.

### Example 8. Production of Fab Fusion Proteins in CHO Cells

Fusion proteins containing a C3 inhibiting nanofitin and/or an HSA binding nanofitin were produced by expression in Chinese Hamster Ovary (CHO) cells. Fab fusion proteins were generated in which the polypeptide having the amino acid sequence of SEQ ID NO: 34 (optionally lacking M at the N terminus and/or K at the C terminus) and/or the polypeptide having the amino acid sequence of SEQ ID NO: 73 were fused to polypeptide chains of a Fab antibody fragment comprising a heavy chain having the amino acid sequence of SEQ ID NO: 36 and a light chain having the amino acid sequence of SEQ ID NO: 35 (ranibizumab (Lucentis®, Genentech)).

The Fab fusion proteins included “Fab NF-E” comprising SEQ ID NO: 59 and SEQ ID NO: 57, “Fab NF-F” comprising SEQ ID NO: 63 and SEQ ID NO: 35, “Fab NF-G” comprising SEQ ID NO: 61 and SEQ ID NO: 60, “Fab NF-H” comprising SEQ ID NO: 63 and SEQ ID NO: 62, “Fab NF-I” comprising SEQ ID NO: 36 and SEQ ID NO: 60, and “Fab NF-J” comprising SEQ ID NO: 58 and SEQ ID NO: 62. Exemplary schematics of the Fab fusion proteins described herein can be found in FIG. 16. The Fab fusion proteins included a flexible linker of 15 amino acids between any two polypeptides (e.g., the antibody chain, the C3 inhibiting Nanofitin, the HSA binding polypeptide) that were part of a longer polypeptide. A secretion signal peptide was located at the N-terminus of each heavy and light chain (cleaved off prior to secretion).

### Example 9: Inhibition of VEGF-Induced Endothelial Cell Proliferation by Fusion Proteins

The effects of the Fab fusion proteins described in Example 8 on induction or inhibition of cellular proliferation was evaluated by using the classical MTT assay, as described previously (see Wang et al., Int J Ophthalmol. 2017; 10(8): 1212-1216). The reduction of tetrazolium salts is widely accepted as a reliable way to examine cell proliferation. The yellow tetrazolium MTT is reduced by metabolically active cells to generate reducing equivalents such as NADH and NADPH. The resulting intracellular purple formazan can be solubilized and quantified by spectrophotometry.

The HUVEC/TERT2 cell line has previously been established by ectopic expression of human telomerase reverse transcriptase (hTERT) in normal umbilical vein endothelial cells. HUVEC/TERT2 cells can be grown for more than 70 population doublings without showing signs of growth retardation and a stable growth rate (by comparison, the parental cell line reaches senescence at 33 doublings). Human hTERT immortalized endothelial cells (HUVEC/TERT2) were seeded into 96-well plates and treated with VEGF (15 ng/mL) together with the Fab fusion proteins described in Example 8 at increasing concentrations. Induction or inhibition of cellular proliferation was measured spectrophotometrically by reduction of MTT.

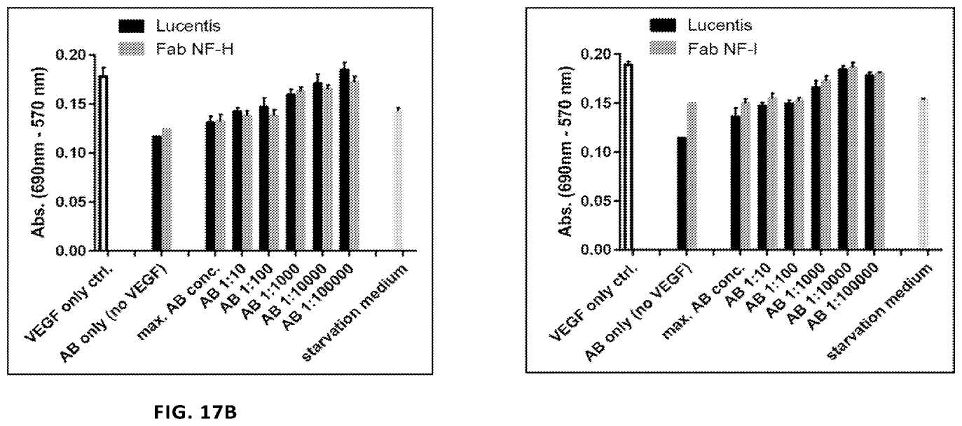

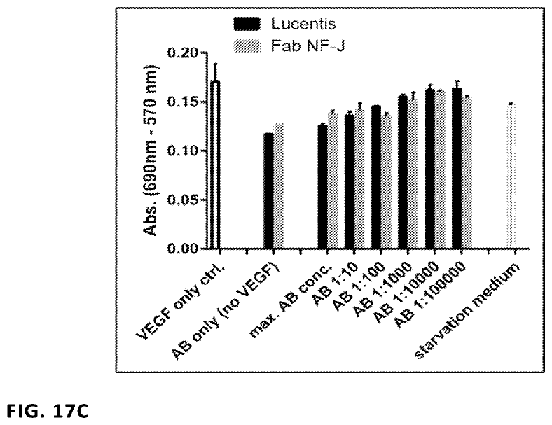

VEGF at a concentration of 15 ng/ml resulted in higher cell numbers compared to treatment with Lucentis® in absence of VEGF. Lucentis® (used as a positive control for inhibiting activity of VEGF) showed a clear and dose dependent effect. The test Fab fusion proteins showed a clear and dose dependent effect on VEGF similar to Lucentis®. Overall, each of the Fab fusion proteins (Fab NF-E, Fab NF-F, Fab NF-H, Fab NF-I, and Fab NF-J) inhibited VEGF-induced proliferation at a level similar to Lucentis®, as shown in FIGS. 17A-17C and Table 21 below.

### Example 10: Kinetic Assays of C3, VEGF, and/or HSA Binding Fusion Proteins

C3 binding, VEGF binding, HSA binding, and equilibrium dissociation constant (K_{D}) measurements were determined by Surface Plasmon Resonance (SPR) using the Sierra SPR-23 instrument. C3 protein was obtained from Complement Technology, Inc. (Cat #A113c). VEGF protein was obtained from Sino Biologicals, Inc. HSA protein was obtained from Sigma-Aldrich, Inc. (Cat #A3782). The Fab fusion proteins described in Example 8 were stored each at 1 mg/mL in PBST, pH 7.4.

Surface plasmon resonance (SPR) requires the attachment of the ligand to a surface and then flowing the analytes across this surface to measure the kinetic rate of the association (kₐ) and the dissociation (k_{d}) of the ligand with the protein. From this measurement, the K_{D} was modeled. Proteins were immobilized on a carboxymethylated dextran biosensor surface by pre-concentrating under various conditions and then amine-coupled to the surface. Upon attachment of the proteins to the surface, experiments with a known binder were performed to confirm that the attached protein retained this ability/activity and the relative coupling efficiencies compared under the various conditions. The polypeptides were evaluated using a serial dilution (typically a 8-point, 3-fold serial dilution assay performed in duplicate). The interaction parameters kₐ, k_{d}, and K_{D} were then determined.

More specifically, the SPR-32 instrument was first cleaned to ensure fluidics were free of any build up. Next, a new biosensor chip was docked and conditioned. Ligand (i.e., C3, VEGF, or HSA) was immobilized to the CM5 biosensor chip surface at a level that resulted in an appropriate theoretically Rmax for the experiment, typically ˜100 RU. This was achieved by taking the ratio of the molecular weights of the ligand/analyte and multiplying by the desired Rmax. The experimentally extrapolated Rmax was then compared to the theoretical Rmax to obtain an estimate of the percent of surface bound protein that is active for binding. A pH-scouting test for C3, VEGF, and HSA was performed at 1 M using 10 mM sodium acetate buffer. Sodium acetate pH 5.0 was observed to give best levels of protein immobilization.

C3, VEGF, and HSA were placed onto Spots B, C, and D, respectively, across all 8 channels to test for the binding of the supplied analytes. Spot A was used as a reference spot. The conditioned biosensor chip was chemically activated with EDC/NHS. Ligands were then injected onto the activated surface to allow for immobilization. After immobilization, 1 M ethanolamine was injected to block any remaining reactive carboxyl groups in the carboxymethylated dextran surface.

In summary, ligands were immobilized under the following conditions: 25° C. on a Sierra Sensors HCA (CM5), Running Buffer of PBST (10 mM Phosphate Buffer pH 7.4, 150 mM NaCl, 0.05% Tween-20; pH 7.4), amine coupling Immobilization Buffer of 10 mM NaOAc pH 5.0, activation with EDC/NHS 7 minutes, immobilization for 2 minutes, and blocking with 1 M ethanolamine for 7 minutes.

The single cycle kinetic method was used to characterize the binding interactions of the proteins. For this method, analyte injections were injected in increasing concentration without regeneration and with shorter dissociation to calculate the association rates. The top concentration was injected at the end, but with longer dissociation time. The dissociation rate was calculated from the top concentration sensorgram, as dissociation rate is independent of the concentration. The signal was observed to create a stair-step pattern, where increasing amounts of analyte remained bound to the surface with each injection. The benefit from this approach is the increase in throughput and the avoidance of regeneration solutions that may be detrimental to the immobilized protein on the surface.

An 8-point, 3-fold serial dilution assay was performed in duplicate for the Fab fusion proteins described in Example 8 starting at 100 nM. Sensorgrams were analyzed using Sierra Sensors Analyzer with a double reference to determine the interaction parameters ka, kd, and K_{D}. The reference channel was first subtracted from the ligand channel as the first reference. The internal blank injections were set to closest blank after injection based on the method. The kd was calculated for the top concentration by fitting the off-rate part of the curve. The kd was then set to global constant for fitting rest of the curves. Rmax was fit locally for each individual curve since the number of available binding sites changed after each injection. Binding data was fit to a 1:1 Langmuir model.

The use of single cycle kinetics method yielded data that fit the 1:1 Langmuir model with low nanomolar affinities for the tested Fab fusion proteins against C3, VEGF, and HSA proteins. Complement inhibiting activity of the affinity matured polypeptides was confirmed in a Wieslab® assay. Table 21 below shows IC50 values and the equilibrium dissociation constants (K_{D}) of the Fab fusion proteins described in Example 8. These results indicate that the Fab fusion proteins described herein have binding activity for C3 and HSA in the low nM range and binding activity for VEGF in the single-digit pM range. Fab NF-H and Fab NF-J were the tightest binders of C3. Fab NF-H and Fab NF-E were the tightest binders of HSA. All of the Fab fusion proteins were found to be active in inhibiting the classical and alternative complement pathways with IC50 values ranging from 47 nM to 273 nM. Fab NF-H and Fab NF-J were the most potent Fab fusion proteins in both the classical pathway and alternative pathway Wieslab® assays. These results indicate that the polypeptides are biologically active in the nM range.

TABLE 21

| | | | | VEGF | | | |
|---|---|---|---|---|---|---|---|
| | | IC50 (nM) | IC50 (nM) | Inhibition | K_{D} (pM) | K_{D} (nM) | K_{D} (nM) |
| | MW | Classical | Alternative | Anti- | Binding | Binding | Binding |
| | (kDa) | Pathway | Pathway | proliferation | to VEGF | to HSA | to C3b |
| Fab NF-E | 64.8 | 65 | 246 | Similar to | ~6 | 3.0 | 1.31 |
| | | | | Lucentis | | | |
| Fab NF-F | 64.9 | 78 | 273 | Similar to | ~6 | 90.6 | 4.09 |
| | | | | Lucentis | | | |
| Fab NF-G | 81.4 | NA | NA | NA | ~6 | 16.9 | 0.21 |
| Fab NF-H | 81.4 | 47 | 123 | Similar to | ~6 | 0.1 | 0.13 |
| | | | | Lucentis | | | |
| Fab NF-I | 64.9 | 63 | 170 | Similar to | 6 | 8.9 | 1.42 |
| | | | | Lucentis | | | |
| Fab NF-J | 73.0 | 48 | 121 | Similar to | ~6 | 16.9 | 0.14 |
| | | | | Lucentis | | | |

NA = Not tested in this assay

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A protein comprising (i) a C3 and/or C3b binding polypeptide and (ii) a VEGF inhibitor, wherein the amino acid sequence of the C3 and/or C3b binding polypeptide has between 4 and 22 substitutions as compared to the Sac7d family consensus sequence of SEQ ID NO: 16, wherein the amino acid sequence of the C3 and/or C3b binding polypeptide comprises amino acids 2-60 of SEQ ID NO: 77, wherein X at positions 2, 3, 4, 6, 15, 17, 18, 31, 37-39, 57, 58, 59 and 61-68 are as set forth below:

| Position | Amino Acid |
|---|---|
| (2) . . . (2) | X is V, A or T |
| (3) . . . (3) | X is T or K |
| (4) . . . (4) | X is - or K |
| (6) . . . (6) | X is R or K |
| (15) . . . (15) | X is E or Q |
| (17) . . . (17) | X is D or E |
| (18) . . . (18) | X is T or I |
| (31) . . . (31) | X is V or I |
| (37) . . . (39) | X X X is EGG, DN- or DQ- |
| (57) . . . (57) | X is M or L |
| (58) . . . (58) | X is Q, D or E |
| (59) . . . (59) | X is M, K, or L |
| (61) . . . (68) | XXXXXXXX is -------K, |
| | EKS--GKK (SEQ ID NO: 82), |
| | EK---QKK (SEQ ID NO: 83), |
| | ARAEREKK (SEQ ID NO: 79), |
| | ARAERE-K (SEQ ID NO: 81), |
| | ARAERE-- (SEQ ID NO: 80), |
| | ARA-EKKK (SEQ ID NO: 84), |
| | E-----KK, ACAEREKK (SEQ |
| | ID NO: 85) or ACA-EKKK |
| | (SEQ ID NO: 86) |

and wherein SEQ ID NO: 77 comprises Y at position 25, W at position 44, and at least one of the following:
E, A, or D at position 8;
A at position 9;
T at position 10;
Y, A, or S at position 22;
Q, N, or A at position 23;
S, V, or T at position 27;
E or D at position 30;
A, I, L, V, Y, M, F or H at position 32;
A, T or S at position 34;
T or I at position 42;
A, I, L, V, Y, M, F or H at position 46; and
S at position 48.

2.-25. (canceled)

26. The protein of claim 1, wherein SEQ ID NO: 77 comprises:
E, A, or D at position 8;
A at position 9;
T at position 10;
Y, A, or S at position 22;
Q, N, or A at position 23;
S, V, or T at position 27;
E or D at position 30;
L at position 32;
A, T or S at position 34;
T or I at position 42;
Y at position 46; and
S at position 48.

27. The protein of claim 1, wherein SEQ ID NO: 77 comprises:
A or D at position 8;
A at position 9;
T at position 10;
A or S at position 22;
N or A at position 23;
V or T at position 27;
D at position 30;
L at position 32;
S at position 34;
I at position 42;
Y at position 46; and
S at position 48.

28.-29. (canceled)

30. The protein of claim 1, wherein SEQ ID NO: 77 comprises:
A at position 8;
A at position 9;
T at position 10;
A at position 22;
N at position 23;
V at position 27;
D at position 30;
L at position 32;
S at position 34;
I at position 42;
Y at position 46; and
S at position 48.

31. The protein of claim 1, wherein SEQ ID NO: 77 comprises:
D at position 8;
A at position 9;
T at position 10;
S at position 22;
A at position 23;
T at position 27;
D at position 30;
L at position 32;
S at position 34;
I at position 42;
Y at position 46; and
S at position 48.

32. The protein of claim 1, wherein the amino acids at positions 2, 3, 4, 6, 15, 17, 18, 31, 37, 38, 39, 57, 58, 59, and 61-68 of SEQ ID NO: 77 are the same as those found at the corresponding position in a protein selected from the group consisting of Sac7d from Sulfolobus acidocaldarius, Sac7e from Sulfolobus acidocaldarius, Sso7d from Sulfolobus solfataricus, Ssh7b from Sulfolobus shibatae, Ssh7a from Sulfolobus shibatae, DBP7 from Sulfolobus tokodaii, Sis7a from Sulfolobus islandicus, Mse7 from Metallosphaera sedula, Mcu7 from Metallosphaera cuprina, Aho7a from Acidianus hospitalis, Aho7b from Acidianus hospitalis, Aho7c from Acidianus hospitalis and Sto7 from Sulfurisphaera tokodaii, when said protein is aligned with SEQ ID NO: 77 with the proviso that the protein may comprise a D17E, N38Q, and/or M59L substitution.

33.-36. (canceled)

37. The protein of claim 1, wherein the C3 and/or C3b binding polypeptide comprises amino acids 2-58 of SEQ ID NO: 33.

38. The protein of claim 1, wherein the C3 and/or C3b binding polypeptide comprises amino acids 2-58 of SEQ ID NO: 34.

39. A protein comprising (i) a C3 and/or C3b binding polypeptide and (ii) a VEGF inhibitor, wherein the C3 and/or C3b binding polypeptide comprises amino acids 2-58 of the sequence MVKVKFXXXGEEKEVXTSKIXXVXRXGKXVXFTYDDXGKXGXGXVXEKDAPKELLD MLARAEREKK (SEQ ID NO: 65), wherein each X independently represents any amino acid, and wherein SEQ ID NO: 65 comprises:
(i) D or E at position 16;
(ii) E or Q at position 37; and
(iii) Y at position 24, W at position 42, or both.

40. The protein of claim 39, wherein SEQ ID NO: 65 comprises Y at position 24 and W at position 42.

41. The protein of claim 40, wherein SEQ ID NO: 65 comprises:
A, I, L, V, Y, M, F or H at position 31;
A, T or S at position 33;
T or I at position 40; and
A, I, L, V, Y, M, F or H at position 44.

42.-58. (canceled)

59. The protein of claim 39, wherein SEQ ID NO: 65 comprises:
E, A, or D at position 7;
A at position 8;
T at position 9;
Y, A, or S at position 21;
Q, N, or A at position 22;
Y at position 24;
S, V, or T at position 26;
E or D at position 29;
A, I, L, V, Y, M, F or H at position 31;
A, T or S at position 33;
T or I at position 39;
W at position 42;
A, I, L, V, Y, M, F or H at position 44; and
S at position 46.

60.-62. (canceled)

63. The protein of claim 39, wherein SEQ ID NO: 65 comprises:
A or D at position 7;
A at position 8;
T at position 9;
A or S at position 21;
N or A at position 22;
Y at position 24;
V or T at position 26;
D at position 29;
L at position 31;
S at position 33;
I at position 40;
W at position 42;
Y at position 44; and
S at position 46.

64.-66. (canceled)

67. The protein of claim 39, wherein SEQ ID NO: 65 comprises:
D at position 7;
A at position 8;
T at position 9;
S at position 21;
A at position 22;
Y at position 24;
T at position 26;
D at position 29;
L at position 31;
S at position 33;
I at position 40;
W at position 42;
Y at position 44; and
S at position 46.

68.-70. (canceled)

71. The protein of claim 39, wherein the C3 and/or C3b binding polypeptide comprises amino acids 2-58 of SEQ ID NO: 33.

72. The protein of claim 39, wherein the C3 and/or C3b binding polypeptide comprises amino acids 2-58 of SEQ ID NO: 34.

73. (canceled)

74. A protein comprising (i) a C3 and/or C3b binding polypeptide comprising amino acids 2-58 of any one of SEQ ID NOs: 22, 23, or 24,
wherein the amino acid sequence comprises D or A at position 7, A at position 8, T at position 9, S or A at position 21, A or N at position 22, Y at position 24, T or V at position 26, D at position 29, L at position 31, S at position 33, I at position 40, W at position 42, Y at position 44, and S at position 46 and (ii) a VEGF inhibitor.

75.-81. (canceled)

82. The protein of claim 1, wherein the VEGF inhibitor comprises an antibody fragment selected from the group consisting of a Fab, F(ab′)₂, Fv, scFv, or a single domain antibody (e.g., a nanobody).

83. The protein of claim 82, wherein the antibody fragment is a Fab comprising a VL domain, a CL domain, a VH domain, and a CH1 domain.

84. The protein of claim 83, comprising the C3 and/or C3b binding polypeptide fused directly or indirectly to one, two, three, or four of:
(i) the VL of the Fab;
(ii) the CL of the Fab;
(iii) the VH of the Fab; or
(iv) the CH1 of the Fab.

85.-86. (canceled)

87. The protein of claim 82, wherein the antibody fragment comprises a heavy chain fragment having the amino acid sequence of SEQ ID NO: 36 and a light chain having the amino acid sequence of SEQ ID NO: 35, optionally wherein the protein comprises (i) SEQ ID NO: 35 and either SEQ ID NO: 38, SEQ ID NO: 40, or SEQ ID NO: 56 or (ii) SEQ ID NO: 36 and either SEQ ID NO: 37, SEQ ID NO: 39, or SEQ ID NO: 55.

88.-94. (canceled)

95. The protein of claim 82, further comprising a half-life extending polypeptide, optionally wherein the protein has a structure depicted in FIG. 11, FIG. 12, FIG. 13, or FIG. 14.

96. A nucleic acid encoding the protein of claim 1.

97.-98. (canceled)

99. A pharmaceutical composition comprising the protein of claim 1 and a pharmaceutically acceptable carrier.

100. A method of treating a subject having or at risk of a complement-mediated disorder, the method comprising administering to the subject a composition comprising an effective amount of the protein of claim 1.

101.-117. (canceled)

118. The method of claim 100, wherein the composition is administered to the eye of a subject suffering from an eye disorder.

119.-136. (canceled)
